(19) United States
(12) Patent Application Publication
(10) Pub. No.: US 2026/0015300 A1
(43) Pub. Date: Jan. 15, 2026

(54) ASSEMBLIES, SYSTEMS, APPARATUSES, AND PROCESSES FOR ENHANCING ALKYLATION PROCESSES

(71) Applicant: Marathon Petroleum Company LP, Findlay, OH (US)

(72) Inventors: Roy Roger Bledsoe, JR., Findlay, OH (US); Rick M. Vice, Findlay, OH (US); Liza M. Pacheco Bernier, Findlay, OH (US); Chuck R. Johnston, Findlay, OH (US); Liana V. Siegel, Findlay, OH (US)

(21) Appl. No.: 19/333,919

(22) Filed: Sep. 19, 2025

Related U.S. Application Data
(63) Continuation-in-part of application No. 17/652,431, filed on Feb. 24, 2022, now Pat. No. 12,461,022.
(60) Provisional application No. 63/153,452, filed on Feb. 25, 2021.
(60) Provisional application No. 63/268,456, filed on Feb. 24, 2022.
(60) Provisional application No. 63/697,044, filed on Sep. 20, 2024.

(51) Int. Cl. C07C 2/80; G05B 17/02

(52) U.S. Cl. CPC C07C 2/80; G05B 17/02

(57) ABSTRACT

Systems, assemblies, apparatuses, and processes to enhance an alkylation process associated with a refining operation may include analyzing samples of an olefin feed and a paraffin feed supplied to one or more alkylation processing units associated with the refining operation, unit materials, and/or a catalyst, via one or more spectroscopic analyzers. The systems and processes further may include prescriptively controlling, via one or more alkylation process controllers, based at least in part on feed properties, unit material properties, and/or catalyst properties, the one or more alkylation processing units, so that the prescriptively controlling results in causing the alkylation process to produce unit materials and/or downstream materials having properties within selected ranges of target properties, thereby to cause the alkylation process to achieve material outputs that more accurately and responsively converge on one or more of the target properties.

### PRIORITY CLAIMS

This application claims priority to and the benefit of U.S. Provisional Application No. 63/697,044, filed Sep. 20, 2024, titled ASSEMBLIES, SYSTEMS, APPARATUSES, AND PROCESSES FOR ENHANCING ALKYLATION PROCESSES, and is a continuation-in-part of U.S. Non-Provisional application Ser. No. 17/652,431, filed Feb. 24, 2022, titled “METHODS AND ASSEMBLIES FOR DETERMINING AND USING STANDARDIZED SPECTRAL RESPONSES FOR CALIBRATION OF SPECTROSCOPIC ANALYZERS,” which claims priority to and the benefit of U.S. Provisional Application No. 63/153,452, filed Feb. 25, 2021, titled “METHODS AND ASSEMBLIES FOR DETERMINING AND USING STANDARDIZED SPECTRAL RESPONSES FOR CALIBRATION OF SPECTROSCOPIC ANALYZERS,” and U.S. Provisional Application No. 63/268,456, filed Feb. 24, 2022, titled “ASSEMBLIES AND METHODS FOR ENHANCING CONTROL OF FLUID CATALYTIC CRACKING (FCC) PROCESSES USING SPECTROSCOPIC ANALYZERS,” the disclosures of all of which are incorporated herein by reference in their entireties.

### TECHNICAL FIELD

The present disclosure relates to assemblies, systems, apparatuses, and processes for enhancing alkylation processes and, more particularly, to assemblies, systems, apparatuses, and processes for enhancing alkylation processes associated with refining operations.

### BACKGROUND

Refining operations may include refinery processes that may be used to produce desired petroleum-based intermediate and final products from hydrocarbon feeds. Many refinery processes are inherently complex, often involving a large number of variables and processing parameters associated with the hydrocarbon feeds and operation of the multiple processing units for transforming the hydrocarbon feed into desired intermediate and final products. Design, control, and optimization of the processing units may benefit from analytical models that describe conversion of hydrocarbon feeds to products. Analytical models, however, may only be useful if provided with timely and accurate information. If the information lacks sufficient accuracy, the analytical model may provide inaccurate outputs, for example, relating to hydrocarbon feedstock monitoring and control, and/or control of the processing units, and resulting products may lack desired properties. If the information is not provided to the analytical model in a sufficiently responsive manner, desired changes based on the information and model outputs may be delayed, resulting in extending the time during which the processes are performed below optimum accuracy and/or efficiency. Conventional laboratory analysis of the hydrocarbon feeds and related materials or processes may suffer from insufficiently responsive results to provide effective monitoring and control of the processes and related materials. For example, off-line laboratory analysis and related modeling studies may involve response times of hours, days, or even weeks, during which processing parameters are not optimized. As a result, the value of such monitoring and control may be reduced when used to monitor and control the processes, for example, during operation.

Although some refinery processes may include devices and strategies for monitoring and controlling the refinery process, Applicant has recognized that such devices and strategies may suffer from delayed acquisition of useful information and/or inaccuracies due to the nature of the devices or strategies. As a result, Applicant has recognized that there may be a desire to provide systems, assemblies, apparatuses, and processes for more accurately monitoring, controlling, and/or optimizing refinery processes and/or for more responsively determining properties and/or characteristics of hydrocarbon feeds, processing unit product materials, intermediate materials, upstream materials, and/or downstream materials related to the refinery processes. Such systems, assemblies, apparatuses, and processes may result in enhanced control and/or optimization of refinery processes for more efficiently producing refinery products.

The present disclosure may address one or more of the above-referenced considerations, as well as other possible considerations.

### SUMMARY

The present disclosure is generally directed to embodiments of systems, assemblies, apparatuses, and processes for enhancing alkylation processes and, more particularly, to assemblies, systems, apparatuses, and processes for enhancing alkylation processes associated with refining operations. Monitoring and control of alkylation processes may be important for producing alkylation-related products having certain characteristics or properties to meet industry standards and/or marketing standards. Using current systems and processes, it may be difficult to achieve desired standards because the systems and methods may suffer from delayed acquisition of useful information and/or inaccuracies due to the nature of the devices or processes. At least some embodiments of the present disclosure may provide assemblies, systems, apparatuses, and/or processes for monitoring, controlling, and/or optimizing alkylation processes, such that the resulting alkylation-related products have desired characteristics or properties that may be achieved more efficiently. In some embodiments, the assemblies, systems, apparatuses, and/or processes disclosed herein may result in acquisition of useful information and/or provide more accurate information for monitoring, controlling, and/or optimizing alkylation processes, in some instances, while the alkylation processes are occurring. This, in turn, may result in producing alkylation-related products having desired characteristics or properties in a more efficient manner. For example, in at least some embodiments, at least some of the acquired information may be used to monitor and prescriptively control alkylation processes, for example, during the alkylation processes, resulting in producing alkylation-related products having desired characteristics or properties in a more efficient manner. For example, prescriptively controlling the alkylation processing assembly and/or the alkylation process, during the alkylation processes, according to some embodiments, may result in causing the alkylation process to produce intermediate materials, unit materials, and/or downstream materials having properties within selected ranges of target properties, thereby to cause the alkylation process to achieve material outputs that more accurately and responsively converge on one or more of the target properties.

In some embodiments, a method for enhancing control of an alkylation process associated with a refining operation may include analyzing, via a first spectroscopic analyzer to provide olefin feed sample spectra, an olefin feed sample of an olefin feed supplied to an alkylation unit positioned to perform at least a portion of the alkylation process, the olefin feed having one or more olefin feed properties. The method further may include analyzing, via one of the first spectroscopic analyzer or a second spectroscopic analyzer to provide paraffin feed sample spectra, a paraffin feed sample of a paraffin feed supplied to the alkylation unit, the paraffin feed having one or more paraffin feed properties. The method further may include predicting one or more olefin feed sample properties associated with the olefin feed based at least in part on the olefin feed sample spectra, and predicting one or more paraffin feed sample properties associated with the paraffin feed based at least in part on the paraffin feed sample spectra.

The method also may include processing, via the alkylation unit, the olefin feed, the paraffin feed, and one or more catalysts to produce one or more corresponding unit materials. The unit materials may include one or more of intermediate materials or unit product materials. The method further may include analyzing a unit material sample of the one or more unit materials via one of the first spectroscopic analyzer, the second spectroscopic analyzer, or a third spectroscopic analyzer, to provide unit material sample spectra, and predicting one or more unit material sample properties associated with the unit material sample based at least in part on the unit material sample spectra.

In some embodiments, the method also may include prescriptively controlling, during the alkylation process, via one or more controllers based at least in part on the one or more olefin feed sample properties, the one or more paraffin feed sample properties, and the one or more unit material sample properties, one or more of: (a) one or more olefin feed properties associated with the olefin feed supplied to the alkylation unit; (b) one or more paraffin feed properties associated with the paraffin feed supplied to the alkylation unit; (c) one or more intermediate properties associated with the intermediate materials produced by the alkylation unit; (d) one or more unit product properties associated with the unit product materials produced by the alkylation unit; (e) operation of the alkylation unit; (f) operation of one or more first processing units positioned upstream relative to the alkylation unit; or (g) operation of one or more second processing units positioned downstream relative to the alkylation unit.

In some embodiments, the prescriptively controlling may cause the alkylation process to produce one or more of: (i) one or more intermediate materials each having one or more properties within a range of one or more target properties of the one or more intermediate materials, (ii) one or more unit product materials each having one or more properties within a range of one or more target properties of the one or more unit product materials, or (iii) one or more downstream materials produced by the one or more second processing units, the one or more downstream materials each having one or more properties within a range of one or more target properties of the one or more downstream materials. The prescriptively controlling may thereby cause the alkylation process to achieve material outputs that more accurately and responsively converge on one or more of the target properties.

In some embodiments, an alkylation process control assembly to enhance control of an alkylation process associated with a refining operation may include a first spectroscopic analyzer positioned to: (a) receive an olefin feed sample of an olefin feed supplied to an alkylation unit associated with the alkylation process, the olefin feed having one or more olefin feed properties, and (b) analyze the olefin feed sample to provide olefin feed sample spectra. The alkylation process control assembly further may include a second spectroscopic analyzer positioned to: (a) receive a paraffin feed sample of a paraffin feed supplied the alkylation unit associated with the alkylation process, the paraffin feed having one or more paraffin feed properties, and (b) analyze the paraffin feed sample to provide paraffin feed sample spectra. The alkylation process control assembly also may include a third spectroscopic analyzer positioned to receive a unit material sample of one or more unit materials produced by the alkylation unit associated with the alkylation process. The one or more unit materials may include one or more of intermediate materials or unit product materials. The unit material sample may be analyzed to provide unit material sample spectra. The first spectroscopic analyzer, the second spectroscopic analyzer, and the third spectroscopic analyzer may be calibrated to, for example, generate standardized spectral responses.

In some embodiments, the alkylation process control assembly further may include an alkylation process controller in communication with the first spectroscopic analyzer, the second spectroscopic analyzer, and the third spectroscopic analyzer. The alkylation process controller may be configured to: (a) predict one or more olefin feed sample properties associated with the olefin feed sample based at least in part on the olefin feed sample spectra, (b) predict one or more paraffin feed sample properties associated with the paraffin feed sample based at least in part on the paraffin feed sample spectra, and (c) predict one or more unit material sample properties associated with the unit material sample based at least in part on the unit material sample spectra. The alkylation process controller also may be configured to prescriptively control, during the alkylation process, based at least in part on the one or more olefin feed sample properties, the one or more paraffin feed sample properties, and the one or more unit material sample properties, one or more of: (a) one or more olefin feed parameters associated with the olefin feed supplied to the alkylation unit; (b) the one or more olefin feed properties associated with the olefin feed supplied to the alkylation unit; (c) one or more paraffin feed parameters associated with the paraffin feed supplied to the alkylation unit; (d) the one or more paraffin feed properties associated with the paraffin feed supplied to the alkylation unit; (e) one or more unit materials properties associated with the one or more unit materials; (f) operation of the alkylation unit; or (g) operation of one or more second processing units positioned downstream relative to the alkylation unit. The prescriptively controlling during the alkylation process may cause the alkylation process to produce one or more of: (a) one or more intermediate materials each having one or more properties within a selected range of one or more target properties of the one or more intermediate materials; (b) one or more unit materials each having one or more properties within a selected range of one or more target properties of the one or more unit materials; or (c) one or more downstream materials each having one or more properties within a selected range of one or more target properties of the one or more downstream materials. The prescriptive control may thereby cause the refining process to achieve material outputs that more accurately and responsively converge on one or more target properties.

In some embodiments, an alkylation process controller to enhance control of an alkylation process associated with a refining operation may be configured to be in communication with one or more spectroscopic analyzers and one or more alkylation processing units. The alkylation process controller may further be configured to: (a) predict one or more olefin feed sample properties associated with an olefin feed sample based at least in part on olefin feed sample spectra generated by the one or more spectroscopic analyzers; (b) predict one or more paraffin feed sample properties associated with a paraffin feed sample based at least in part on paraffin feed sample spectra generated by the one or more spectroscopic analyzers; and (c) predict one or more unit material sample properties associated with a unit material sample based at least in part on unit material sample spectra generated by the one or more spectroscopic analyzers. The one or more unit material sample properties may be associated with one or more unit materials produced by the one or more alkylation processing units, and the one or more unit materials may include one or more of intermediate materials or unit product materials.

The alkylation process controller also may be configured to prescriptively control, during the alkylation process, based at least in part on the one or more olefin feed sample properties, the one or more paraffin feed sample properties, and the one or more unit material sample properties, one or more of: (a) the one or more olefin feed properties associated with the olefin feed supplied to the one or more alkylation processing units; (b) the one or more paraffin feed properties associated with the paraffin feed supplied to the one or more alkylation processing units; (c) one or more catalyst feed properties associated with a catalyst feed supplied to the one or more alkylation processing units; (d) one or more intermediate properties associated with the intermediate materials produced by one or more of the alkylation processing units; (e) operation of the one or more alkylation processing units; (f) one or more unit materials properties associated with the one or more unit materials produced by the one or more alkylation processing units; or (g) operation of one or more second processing units positioned downstream relative to the one or more alkylation processing units.

The prescriptively controlling may thereby cause the refining process to produce one or more of: (a) the one or more intermediate materials each having one or more properties within a selected range of one or more target properties of the one or more intermediate materials, (b) the one or more unit materials each having one or more properties within a selected range of one or more target properties of the one or more unit materials, or (c) one or more downstream materials produced by the second processing units each having one or more properties within a selected range of one or more target properties of the one or more downstream materials. The prescriptively controlling may thereby cause the refining process to achieve material outputs that more accurately and more responsively converge on one or more of the target properties.

In some embodiments, a method for enhancing control of an alkylation process associated with a refining operation may include predicting one or more target properties of one or more unit materials produced by the alkylation process, and predicting, during a first time cycle a first portion of one or more of: (a) one or more first olefin feed sample properties associated with a first olefin feed sample based at least in part on first olefin feed sample spectra generated by one or more spectroscopic analyzers; (b) one or more first paraffin feed sample properties associated with a first paraffin feed sample based at least in part on first paraffin feed sample spectra generated by the one or more spectroscopic analyzers; or (c) one or more first unit material sample properties associated with a first unit material sample based at least in part on first unit material sample spectra generated by the one or more spectroscopic analyzers. The unit material sample properties may be associated with the one or more unit materials produced by the alkylation process, and the one or more unit materials may include one or more of intermediate materials or unit product materials.

The method also may include determining a first set of differences during the first time cycle. The first set of differences may include differences between (a) the one or more target properties of the one or more unit materials produced by the alkylation process and (b) the one or more first unit material sample properties associated with the first unit material sample. The method further may include generating one or more first control signals indicative of one or more process parameters associated with the alkylation process based at least in part on the first set of differences during the first time cycle.

In some embodiments, the method further may include controlling, during the first time cycle, and based at least in part on the one or more first control signals, one or more of: (a) one or more olefin feed properties associated with the olefin feed; (b) one or more paraffin feed properties associated with the paraffin feed; (c) one or more catalyst feed properties associated with a catalyst feed of the alkylation process; (d) one or more intermediate properties associated with the intermediate materials produced by the alkylation process; (e) the one or more process parameters associated with operation of the alkylation process; (f) one or more process parameters associated with operation of one or more first processing units positioned upstream relative to the alkylation process; or (g) one or more process parameters associated with operation of one or more second processing units positioned downstream relative to the alkylation process. The controlling may cause the alkylation process to achieve material outputs that more accurately and responsively converge on the one or more target properties of the one or more unit materials produced by the alkylation process.

The method also may include predicting, during a second time cycle after the first time cycle, one or more of: (a) one or more second olefin feed sample properties associated with a second olefin feed sample based at least in part on second olefin feed sample spectra generated by the one or more spectroscopic analyzers, (b) one or more second paraffin feed sample properties associated with a second paraffin feed sample based at least in part on second paraffin feed sample spectra generated by the one or more spectroscopic analyzers, or (c) one or more second unit material sample properties associated with a second unit material sample based at least in part on second unit material sample spectra generated by the one or more spectroscopic analyzers.

The method further may include determining, during the second time cycle, a second set of differences between (a) the one or more target properties of the one or more unit materials produced by the alkylation process and (b) the one or more second unit material sample properties associated with the second unit material sample. The method also may include generating, during the second time cycle, one or more second control signals indicative of one or more process parameters associated with the alkylation process based at least in part on the second set of differences.

In some embodiments, the method further may include controlling, during the second time cycle, based at least in part on the one or more second control signals, one or more of: (a) the one or more olefin feed properties associated with the olefin feed; (b) the one or more paraffin feed properties associated with the paraffin feed; (c) the one or more catalyst feed properties associated with the catalyst feed; (d) the one or more intermediate properties associated with the intermediate materials produced by the alkylation process; (e) the one or more process parameters associated with operation of the alkylation process; (f) the one or more process parameters associated with operation of one or more first processing units positioned upstream relative to the alkylation process; or (g) the one or more process parameters associated with operation of one or more second processing units positioned downstream relative to the alkylation process. The controlling may be iterative between the one or more first control signals, the one or more second control signals, and one or more nth control signals. The controlling may thereby cause the alkylation process to achieve material outputs that more accurately and responsively converge on the one or more target properties of the unit materials produced by the alkylation process.

In some embodiments, an alkylation processing assembly for performing an alkylation process associated with a refining operation may include one or more alkylation processing units including one or more of a reactor, an acid settler, an isostripper, a depropanizer, a deisobutanizer, or a debutanizer. The alkylation processing assembly also may include a first spectroscopic analyzer positioned to: (a) receive an olefin feed sample of an olefin feed supplied to the one or more alkylation processing units, the olefin feed having one or more olefin feed properties, and (b) analyze the olefin feed sample to provide olefin feed sample spectra. The alkylation process control assembly further may include a second spectroscopic analyzer positioned to: (a) receive a paraffin feed from a paraffin feed supplied the one or more alkylation processing units, the paraffin feed having one or more paraffin feed properties, and (b) analyze the paraffin feed sample to provide paraffin feed sample spectra. The alkylation process control assembly also may include a third spectroscopic analyzer positioned to: (a) receive a unit material sample of one more unit materials produced by the one or more alkylation processing units associated with the alkylation process, and (b) analyze the unit material sample to provide unit material sample spectra. The one or more unit materials may include one or more of intermediate materials or unit product materials. The first spectroscopic analyzer, the second spectroscopic analyzer, and the third spectroscopic analyzer may be calibrated to generate standardized spectral responses.

In some embodiments, the alkylation process control assembly further may include an alkylation process controller in communication with the first spectroscopic analyzer, the second spectroscopic analyzer, and the third spectroscopic analyzer. The alkylation process controller may be configured to: (a) predict one or more olefin feed sample properties associated with the olefin feed sample based at least in part on the olefin feed sample spectra, (b) predict one or more paraffin feed sample properties associated with the paraffin feed sample based at least in part on the paraffin feed sample spectra, and (c) predict one or more unit material sample properties associated with the unit material sample based at least in part on the unit material sample spectra. The alkylation process controller also may be configured to prescriptively control, during the alkylation process, based at least in part on the one or more olefin feed sample properties, the one or more paraffin feed sample properties, and the one or more unit material sample properties, one or more of: (i) one or more olefin feed parameters associated with the olefin feed supplied to the one or more alkylation processing units; (ii) the one or more olefin feed properties associated with the olefin feed supplied to the one or more alkylation processing units; (iii) one or more paraffin feed parameters associated with the paraffin feed supplied to the one or more alkylation processing units; (iv) the one or more paraffin feed properties associated with the paraffin feed supplied to the one or more alkylation processing units; (v) one or more intermediate properties associated with the intermediate materials produced by the one or more alkylation processing units; (vi) one or more unit materials properties associated with the one or more unit materials; (vii) operation of the one or more alkylation processing units; or (viii) operation of one or more second processing units positioned downstream relative to the one or more alkylation processing units.

In some embodiments, the prescriptively controlling may thereby cause the refining process to produce one or more of: (aa) one or more intermediate materials each having one or more properties within a selected range of one or more target properties of the one or more intermediate materials; (bb) one or more unit materials each having one or more properties within a selected range of one or more target properties of the one or more unit materials; or (cc) one or more downstream materials each having one or more properties within a selected range of one or more target properties of the one or more downstream materials. The alkylation process may thereby achieve material outputs that more accurately and responsively converge on one or more of the target properties.

Still other aspects, examples, and advantages of these exemplary aspects and embodiments, are discussed in more detail below. It is to be understood that both the foregoing information and the following detailed description are merely illustrative examples of various aspects and embodiments and are intended to provide an overview or framework for understanding the nature and character of the claimed aspects and embodiments. Accordingly, these and other objects, along with advantages and features of the present disclosure herein disclosed, may become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the exemplary embodiments discussed herein and the various ways in which they may be practiced. According to common practice, the various features of the drawings discussed below are not necessarily drawn to scale. Dimensions of various features and elements in the drawings may be expanded or reduced to illustrate more clearly one or more of the embodiments of the disclosure.

FIG. 1 is a schematic diagram of an example refinery operation including an example alkylation process and other example processes, according to embodiments of the disclosure.

FIG. 2A is a schematic diagram of an example alkylation process including example spectroscopic analyzers for predicting sample properties of materials associated with the alkylation process, according to embodiments of the disclosure.

FIG. 2B is a schematic diagram of another example alkylation process including example spectroscopic analyzers for predicting sample properties of materials associated with the alkylation process, according to embodiments of the disclosure.

FIG. 3 is a schematic block diagram of an example alkylation process including an example assembly for enhancing control of the alkylation process, according to embodiments of the disclosure.

FIG. 4 is a schematic block diagram of an example sample conditioning assembly, according to embodiments of the disclosure.

FIG. 5A is a graph depicting example sample spectra of multiple raw crude feedstocks obtained from example spectroscopic analyzers, according to embodiments of the disclosure.

FIG. 5B is a graph depicting a portion of the graph of FIG. 5A, according to embodiments of the disclosure.

FIG. 5C is a graph depicting example sample spectra of an alkylate product obtained from example spectroscopic analyzers, according to embodiments of the disclosure.

FIG. 5D is a graph depicting the second derivative of the graph of FIG. 5C, according to embodiments of the disclosure.

FIG. 6A is a block diagram of an example method to enhance an alkylation process associated with a refining operation, according to embodiments of the disclosure.

FIG. 6B is a continuation of the block diagram shown in FIG. 6A, according to embodiments of the disclosure.

FIG. 6C is a continuation of the block diagram shown in FIG. 6A and FIG. 6B, according to embodiments of the disclosure.

FIG. 6D is a continuation of the block diagram shown in FIG. 6A, FIG. 6B, and FIG. 6C, according to embodiments of the disclosure.

FIG. 6E is a continuation of the block diagram shown in FIG. 6A, FIG. 6B, FIG. 6C, and FIG. 6D, according to embodiments of the disclosure.

FIG. 7A is a block diagram illustrating an example process for enhancing an alkylation process, according to embodiments of the disclosure.

FIG. 7B is a continuation of the block diagram shown in FIG. 7A, according to embodiments of the disclosure.

FIG. 7C is a continuation of the block diagram shown in FIG. 7A and FIG. 7B, according to embodiments of the disclosure.

FIG. 8 is a block diagram illustrating another example process for enhancing an alkylation process, according to embodiments of the disclosure.

FIG. 9A is an example graph showing an example alkylate property as a function of time during an example alkylation process, according to embodiments of the disclosure.

FIG. 9B is an example graph showing another example alkylate property as a function of time during an example alkylation process, according to embodiments of the disclosure.

FIG. 10 is a schematic diagram of an example alkylation process controller configured to at least partially control an alkylation processing assembly, according to embodiments of the disclosure.

### DETAILED DESCRIPTION

Referring now to the drawings in which like numerals indicate like parts throughout the several views, the following description is provided as an enabling teaching of exemplary embodiments, and those skilled in the relevant art will recognize that many changes may be made to the embodiments described. It also will be apparent that some of the desired benefits of the embodiments described may be obtained by selecting some of the features of the embodiments without utilizing other features. Accordingly, those skilled in the art will recognize that many modifications and adaptations to the embodiments described are possible and may even be desirable in certain circumstances. Thus, the following description is provided as illustrative of the principles of the embodiments and not in limitation thereof.

The phraseology and terminology used herein is for the purpose of description and should not be regarded as limiting. For example, terms such as “spectroscopic analyzer,” “analyzer,” and “spectrometer” may be used interchangeably. As used herein, the term “plurality” refers to two or more items or components. A multi-component sample may refer to a single (one) sample including a plurality of components, such as two or more components. The terms “comprising,” “including,” “carrying,” “having,” “containing,” and “involving,” whether in the written description or the claims and the like, are open-ended terms, for example, to mean “including but not limited to,” unless otherwise stated. Thus, the use of such terms is meant to encompass the items listed thereafter, and equivalents thereof, as well as additional items. The transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not necessarily, by itself, connote any priority, precedence, or order of one claim element over another or the temporal order in which acts of a method are performed, but are used merely as labels to distinguish one claim element having a certain name from another element having a same name (but for use of the ordinal term) to distinguish claim elements.

Certain terminology used herein may have definitions provided for the purpose of illustration and not limitation. For example, as used herein, the “sampling circuit” may refer to an assembly for facilitating separation of a sample of a material, a sample of a composition of material, and/or a sample of a product from a refining process for removal and/or analysis of the sample. Similarly, the term “sample conditioner” may refer to an assembly for facilitating preparation of a sample for analysis, for example, to improve the accuracy of analysis of the sample and/or to provide consistency and/or repeatability of the analysis of the sample or more than one sample. Additionally, the term “sample stream” may refer to a portion of a sample stream supplied to one or more spectroscopic analyzers for spectroscopic analysis by the one or more spectroscopic analyzers. As used herein, the term “sample introducer” may refer to a component or assembly that may be used to facilitate the provision of a conditioned sample (portion or stream) to one or more spectroscopic analyzers for analysis. As used herein, the term “sample probe” may refer to a component or an interface used to facilitate collection of a sample for analysis by, for example, one or more spectroscopic analyzers. As used herein, the term “analyzer probe” may refer to a component of one or more spectroscopic analyzers that facilitates direction of electromagnetic radiation (e.g., light energy) from a source through a sample stream (e.g., a conditioned sample stream) to detect and/or measure one or more of absorbance, transmittance, transflectance, reflectance, attenuated total reflectance (ATR), or scattering intensity associated with the sample stream.

As used herein, the term “spectroscopic analyzer” may refer an analyzer that may be used to measure or predict one or more properties of a sample of, for example, a material, a composition of materials, and/or a refinery product (an intermediate product or an end product). In some embodiments, the spectroscopic analyzers may be used on-line or in a laboratory setting. “Spectroscopic analyzer” may refer in some instances to a spectroscopic analyzer assembly, which may include a spectroscopic analyzer and an analyzer controller in communication with one or more spectroscopic analyzers. The analyzer controller may be configured for use with a corresponding spectroscopic analyzer for pre-processing and/or post-processing steps or procedures related to a spectroscopic analysis, as will be understood by those skilled in the art. In some embodiments, the analyzer controller may be physically connected to the spectroscopic analyzer. In some such embodiments, the spectroscopic analyzer may include a housing, and at least a portion of the analyzer controller may be contained in the housing. In some embodiments, the analyzer controller may be in communication with the spectroscopic analyzer via a hard-wired communications link and/or wireless communications link. In some embodiments, the analyzer controller may be physically separated from the spectroscopic analyzer and may be in communication with the spectroscopic analyzer via a hard-wired communications link and/or a wireless communications link. In some embodiments, physical separation may include being spaced from one another, but within the same building, within the same facility (e.g., located at a common manufacturing facility, such as a refinery), or being spaced from one another geographically (e.g., anywhere in the world). In some physically separated embodiments, both the spectroscopic analyzer and the analyzer controller may be linked to a common communications network, such as a hard-wired communications network and/or a wireless communications network. Such communications links may operate according to any known hard-wired communications protocols and/or wireless communications protocols, as will be understood by those skilled in the art.

As used herein, the term “on-line” may refer to equipment and/or processes that are physically located at or adjacent to processing assemblies during operation and, for at least some embodiments, may be capable of providing real-time and/or near real-time analysis and/or data capable of real-time and/or near real-time analysis. For example, in some embodiments, an on-line spectroscopic analyzer may receive one or more sample streams directly from a processing assembly or process and analyze the one or more sample streams in real-time or near real-time to provide results that may, in some embodiments, be used to at least partially control operation of one or more processing assemblies and/or one or more processes in real-time or near real-time. In some embodiments, the on-line spectroscopic analyzer or analyzers may be physically located in a laboratory setting. This may be either extractive (e.g., a sample stream is drawn off of a processing unit and supplied to a spectroscopic analyzer and/or to one or more sensors) or in situ (e.g., a probe of a spectroscopic analyzer or one or more sensors is present in a conduit associated with the processing assembly).

As used herein, the term “at-line” may refer to equipment and/or processes that are physically located at or adjacent to processing assemblies during operation, but which, for at least some embodiments, are not capable of providing real-time and/or near real-time analysis and/or are not capable providing data capable of real-time and/or near real-time analysis. For example, in an “at-line” process, a “field analyzer” located physically at or adjacent a processing assembly may be used to analyze a sample withdrawn from the processing assembly or process and manually taken to the field analyzer for analysis. In some embodiments, the at-line spectroscopic analyzer or analyzers may be physically located in a laboratory setting. For example, in some embodiments, an at-line spectroscopic analyzer would not receive a sample stream directly from processing assemblies, but instead, would manually receive a sample manually withdrawn from a processing unit by an operator and manually taken or delivered by the operator to the at-line spectroscopic analyzer.

As used herein, the term “predicting” may refer to measuring, estimating, determining, and/or calculating one or more properties of a material, a composition of materials, and/or a refinery product (e.g., an intermediate product or an end product) based on, for example, a mathematical relationship, a correlation, an analytical model, and/or a statistical model.

Certain terminology used herein to describe a flow or material, such as a “stream,” a “feedstock,” “products,” and the like may be used interchangeably and are not meant to be limiting. A strict literal definition of these terms may not be appropriate as a standard definition because, for example, a particular flow may change depending on the viewpoint perspective used within the refining process. In some embodiments, it may be understood that a flow referred to as a “product” produced by a certain refining process and/or processing unit, for example, may simultaneously be viewed as a “feedstock” input for another refining process and/or processing unit. Similarly, a “stream” may refer to a flow into, out of, between, or internal to a certain refining process and/or processing unit.

The refining process may use a plurality of processes and/or processing units to divide, combine, or alter various hydrocarbon streams in the production of end products, performing processes such as distillation, catalytic reforming, catalytic cracking, hydrotreating, alkylation, isomerization, and/or other processes. The products from these processes may provide, for example, gas (liquified petroleum gas), naphtha, aviation fuel, motor fuel, and/or other feedstocks for petrochemical industries. The predicted yields and quality of these components, however, typically require detailed knowledge of the properties of the feedstock and input and output streams, including, for example, physical properties, chemical composition, the molecular weight of constituents, and/or other properties, which often require laboratory analysis to obtain a result.

In some embodiments disclosed herein, spectroscopic analyzers may be used to non-invasively predict (or determine) properties and/or related information associated with materials associated with a refining operation. The systems and methods disclosed herein may utilize spectroscopic analyzers and/or other instrumentation to predict properties of hydrocarbons and other materials, and the properties may be used, either alone or in combination with additional information, as control variables to enhance operation and/or control of processing units and/or associated processes within a refining operation. For example, spectroscopic analyzers may predict the properties of hydrocarbon samples, including raw crude oil, fractions of crudes, and/or products or compound material streams associated with individual refining processes and/or processing units. An alkylation process, for example, may receive input streams from one or more other refining processes and produce alkylate products, for example, for blending of gasoline having target product specifications, including, for example, octane specifications. In some embodiments, one or more spectroscopic analyzers may be linked to one or more signal processing devices to permit numerical treatment of spectra generated by the one or more spectroscopic analyzers, and, in at least some embodiments, the treated spectra may be communicated to one or more controllers configured to use the treated spectra to determine control signals for controlling one or more aspects of the alkylation process. In some embodiments, the assemblies, systems, apparatuses, and processes may be used in an on-line manner for real-time data interpretation and may provide results with accuracy at least similar to that of other primary test methods, but in a comparatively more responsive manner. Non-on-line analysis is also contemplated.

The spectroscopic analyzers may vary by type, or by the anticipated content of the stream being sampled. The spectroscopic analyzers may be, for example, one or more near-infrared (NIR) spectroscopic analyzers, one or more mid-infrared (mid-IR) spectroscopic analyzers, a combination of one or more NIR and mid-IR spectroscopic analyzers, one or more Raman spectroscopic analyzers, or one or more nuclear magnetic resonance (NMR) spectroscopic analyzers.

In some refining operations, raw crude may be supplied to a refinery and may be conveyed by one or more crude pumps to refinery processing units for converting the raw crude into desired intermediate and final products. For example, the raw crude may be conveyed to refinery processing units, such as, for example, a crude distillation unit, a vacuum distillation unit, a gas plant unit, and/or other refinery processing units performing, for example, treatment processes, thermal separation processes, hydrogen-treating processes, and/or cracking processes. The content and properties of different supplies of the raw crude may vary significantly, depending on, for example, the supplier and/or even different batches from the same supplier. As a result, it may be desirable to subject the different supplies of raw crude to different processes and/or different processing conditions in order to improve yields and/or efficiencies associated with operation of the refinery. Thus, determining the content and/or properties of the raw crude and/or the content and/or properties of the intermediate products or final products may be desirable in order to control one or more of the refinery processing units to promote more efficient operation of the refinery. For example, according to some embodiments, the raw crude, intermediate products, and/or final products may be analyzed to determine content and/or properties, and the results of the analysis may be used to at least partially control the refinery operation, including, for example, alkylation processes, thereby to enhance the alkylation processes. In some embodiments, such analysis may be conducted in real-time, or near-real-time, for example, such that adjustments may be made to (1) parameters and/or properties associated with the raw crude, the intermediate materials, and/or the final products, and/or (2) parameters and/or operating characteristics associated with one or more of the refinery processing units, such as, for example, processing units associated with the alkylation processes. In some embodiments, real-time, or near-real-time, analysis may result in more effective and/or more responsive adjustments may be performed, thereby resulting in enhanced alkylation processes, which may improve efficiencies associated with the refinery operation. For example, real-time, or near-real-time, analysis of the raw crude supplied to the refinery may facilitate more effective control of various processes and/or prioritization of production, for example, at least in part in response to demand, production targets, and/or other factors.

According to some embodiments, alkylation processes may receive materials from other (e.g., upstream) refinery operations (or supplies) and may use those materials to produce alkylate, which may include high-octane hydrocarbons for blending in motor gasoline and aviation gasoline. These high-octane hydrocarbons may be blended with gasoline to increase octane ratings associated with the gasoline. In some examples of an alkylation operation, olefins (e.g., C3, C4, C5) may be combined with paraffins to form iso-paraffins. For example, isobutane may be combined with (or alkylated with) isobutene or normal butene to form iso-octane, sometimes in the presence of a catalyst, such as an acid catalyst, in some instances, at relatively lower temperatures and/or relatively lower pressures, to produce the alkylate and other materials. Olefins are a class of hydrocarbons made up of one or more pairs of carbon atoms linked by a carbon-carbon double bond. The alkylate produced may provide a premium gasoline blending stock because it may be relatively cleaner burning and may have strong antiknock properties. The octane number of the alkylate may depend on the alkenes used and the operating conditions of the alkylation unit and other processing units. Some alkylate hydrocarbon products may have, for example, research octane numbers (RON) ranging from 90 to 98 and may be relatively free from undesirable aromatics, benzene, and sulfur. Some alkylate products may have boiling points in the gasoline boiling point range, but may have low volatility, such that they may be used as additives while still meeting stringent volatility specifications. Some alkylation processes may result in relatively reduced aromatics, for example, to comply with relatively tighter aromatics limits on gasoline associated with different fuel specifications for different markets. Some alkylation processes may also be useful, for example, for several very light olefins produced during other refining processes.

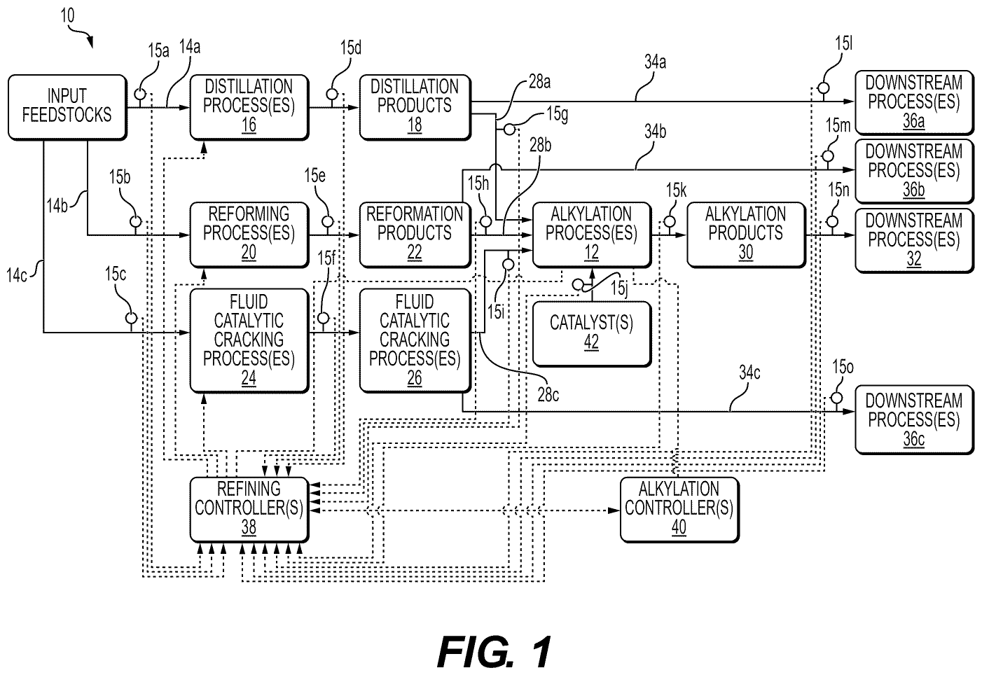

FIG. 1 is a schematic block diagram of an example refinery operation 10 including example alkylation process(es) 12, as well as other example processes, according to embodiments of the disclosure. As shown in FIG. 1, the refinery operation 10 may receive several input feedstocks, for example, feeds 14a, 14b, and 14c, which may be subjected to different refining processes in order to provide intermediate products used as inputs to additional or downstream refining processes or which may be collected as final products for additional uses or delivery, either at the location of the refinery operation 10 or off-site. For example, input feedstock 14a may be supplied to one or more distillation process(es) 16, resulting in one or more distillation products 18. As shown, in some embodiments, input feedstock 14b may be supplied to one or more reforming processes 20, resulting in one or more reformer products 22 (e.g., reformate). In some embodiments of the refinery operation 10, input feedstock 14c may be supplied to one or more fluidized catalytic cracking (FCC) process(es) 24, resulting in one or more FCC products 26. It is contemplated that the input feedstocks 14a, 14b, and/or 14c may be supplied from a common source or different sources and may include the same components and/or may have the same characteristics or may include different components and/or may have different characteristics.

In some embodiments, the example alkylation process(es) 12 may have input feedstocks 28a, 28b, and 28c from processing units associated with refining processes (e.g., distillation process(es) 16, reforming process(es) 20, and/or FCC process(es) 24) upstream of the alkylation process 12. Input feedstocks 28a, 28b, and 28c are shown as examples, and a lesser or greater number of flows (e.g., 28d, 28e, . . . 28n) into the alkylation process(es) 12 may also be contemplated from one or more other refining process(es) or processing units. Input feedstocks for the alkylation process(es) 12 may also include one or more catalyst(s) 42 to facilitate reactions in the alkylation process. The various input feedstocks may have different contents or properties, or one or more of the feedstocks may include substantially the same or similar contents or properties. In some examples, one or more of the feedstocks may have substantially similar contents or properties but of differing quality or purity. The alkylation process(es) 12 may produce one or more alkylation products 30 that may provide feedstocks for other downstream processes 32 or may be separated for sale or use as finished or semi-finished products.

Other products 30a, 30b, and 30c produced by the distillation process(es) 16, reforming process(es) 20, and/or FCC process(es) 24, respectively, may also provide feedstocks for other downstream processes 32a, 32b, and 32c respectively, and/or may be separated for sale and/or use as finished or semi-finished products (e.g., excess hydrogen, propane, and/or gasoline blending components).

The refinery operation 10 may include one or more refining controller(s) configured to receive inputs and control aspects of the refinery to meet operational targets 38. The one or more refining controller(s) 38 may, for example, receive signals from one or more spectroscopic analyzers 15a, 15b, 15c, . . . 15n positioned to monitor properties and/or contents of various refinery streams. The one or more spectroscopic analyzers 15a-15n may be configured to generate sample spectra of material samples and predict properties and/or contents of the samples based at least in part on the spectra. For example, the one or more refining controllers 38 may receive signals from the one or more spectroscopic analyzers 15a-15n, including spectroscopic analyzers 15a, 15b, and 15c, which may be positioned to monitor input feedstocks 14a, 14b, and 14c, respectively, and generate control signals to adjust one or more process parameters of the refinery operation 10. Additionally, one or more alkylation controller(s) 40 may be in communication with the one or more refining controller(s) 38 (and/or the one or more spectroscopic analyzers 15a-15n) and may be configured to monitor properties and/or contents of various input feeds, unit materials, intermediate materials, and/or finished product streams associated with the alkylation process(es) 12. In some embodiments, the one or more alkylation controller(s) 40 may be configured to generate control signals that cause the alkylation process(es) 12 to achieve material outputs that more accurately and responsively converge on one or more preselected target properties.

In some embodiments, as shown in FIG. 1, the refinery operation 10 may include one or more distillation process(es) 16. For example, the one or more distillation process(es) 16 may include performing distillation, for example, via one or more reactor towers of a crude distillation unit. The one or more distillation process(es) 16 may include supplying a hydrocarbon feed (e.g., input feedstock 14a) to one or more crude distillation units, which may be configured to produce fractionated distillation products 18, which may be used to provide feed materials for one or more of the refining processes associated with the refinery operation 10, such as, for example, one or more reforming process(es) 20, one or more FCC process(es) 24, and/or one or more alkylation process(es) 12. At least some of the distillation products 18 may also be supplied as a feedstock 34a for one or more downstream processes 36a.

Depending on the processing parameters used in the operations of the distillation process(es) 16, the distillation products 18 may vary in purity and quality. Distillation products 18 may be treated by, for example, a hydrotreater to remove sulfur and other contaminants prior to being supplied to other processing units to improve the quality of final products and/or reduce corrosion in downstream processing units. In some embodiments, distillation products 18a may produce input feedstock 28a for the alkylation process(es) 12 including isobutane produced during an isomerization process, high-purity isobutane (e.g., light distillate streams of greater than about 95% isobutane originating from a gas plant unit), other materials, or a combination thereof.

In some embodiments, the reforming process(es) 20 may include a catalytic reforming process, which may receive input feedstock 14b for converting naphtha into high-octane reformate (e.g., RON ranging from, for example, about 97 to about 104) and produce reformation products 22. At least a portion of the reformation products 22 may be used as an input feedstock 28b to the alkylation process(es) 12. At least some of the reformation products 22 may also be supplied as a feedstock 34b for one or more downstream processes 36b. Input feedstock 28b for the alkylation process(es) 12 may be a paraffin feed for an alkylation reaction. The paraffin feed may include, for example, isobutane and/or isopentane. Input feedstock 28b may include, for example, lower-purity isobutane (e.g., including less than about 70% isobutane by volume), isopentane, and/or other components. Input feedstock 28b may also be treated, for example, by a hydrotreater, prior to being supplied to the alkylation process(es) 12.

In some embodiments, the FCC process(es) 24 may use a fluid catalytic cracking unit (FCCU) to process input feedstock 14c to produce FCC products 26. FCC products 26 may include input feedstock 28c for the alkylation process(es) 12 in the refinery operation 10. Input feedstock 28c may include, for example, one or more olefin feeds to the alkylation process(es) 12. Alternatively, or in addition, at least some of the one or more olefin feeds for the alkylation process(es) 12 may come from a Coker unit, a polyolefin unit, and/or an ether unit. Olefins in input feedstock 28c may include, for example, some or all of ethylene, propylene, pentalene, butylene, and amylene. In some examples, the olefin feed may additionally include paraffins and isoparaffins in the C3-C5 range.

The one or more olefin and paraffin feeds for the alkylation process(es) 12 may vary in quality and may also include, for example, diluents (such as propane, n-butane, and n-pentane), non-condensables (e.g., such as ethane and/or hydrogen), and contaminants. Diluents may have no effect on the chemistry of the alkylation process(es) 12, but they may occupy a portion of the reactors of an alkylation unit and may influence the yield of secondary reactions (e.g., of polymerization and undesired organofluoride side products). Non-condensables may be similar to diluents, but may not condense at the pressures and temperatures of the alkylation process, and therefore, they may concentrate to a point that they need to be vented or otherwise removed.

Contaminants in the olefin feed, paraffin feed, and/or other feeds may include compounds that may react with and/or dilute the one or more catalyst(s) 42. Contaminants may increase catalyst consumption in the alkylation process(es) 12 and may contribute to the production of undesirable reaction products and polymeric formations. Contaminants may include, for example, free water, mercaptan sulfur, diolefins, methanol, and/or ethanol. Oxygenated compounds and free water present in the feed may dilute the catalyst and reduce alkylate quality. Diolefins, for example, may lead to more rapid consumption of the catalyst and reduce alkylate octane. In some embodiments, the input feedstocks to the alkylation process(es) 12 may be monitored for purity and the presence/concentration of diluents, non-condensables, and contaminants (for example, with measurement devices, such as spectroscopic analyzers). In some embodiments, on-line control may be used to adjust the olefin feed and/or the other feedstocks to improve and/or optimize octane and yield of products from the alkylation process(es) 12, for example, by adjusting isoparaffin/olefin ratio and/or other hydrocarbon parameters, while also adjusting temperature, catalyst to hydrocarbon ratio, and/or other process parameters of alkylation.

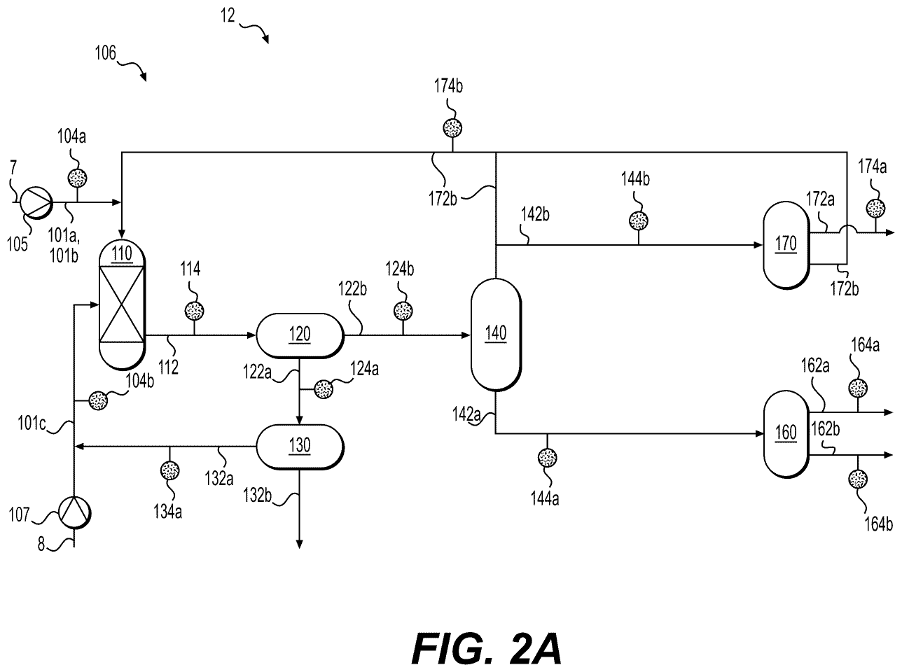

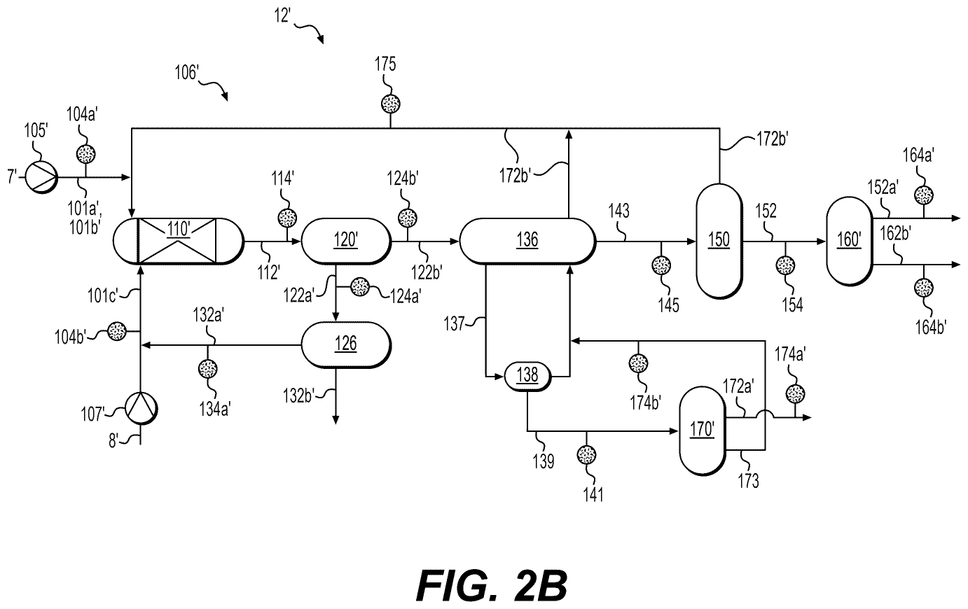

FIG. 2A and FIG. 2B are schematic block diagrams of example alkylation process(es) 12, including example spectroscopic analyzers as measurement devices for predicting sample properties of materials associated with the alkylation process(es) 12, according to embodiments of the disclosure. FIG. 2A depicts an example alkylation process using hydrofluoric acid as a catalyst, and FIG. 2B depicts an example alkylation process using sulfuric acid as a catalyst.

As shown in FIG. 2A, one or more hydrocarbon feeds 7 may be supplied by one or more pumps 105 to an alkylation unit 106 of the alkylation process(es) 12. The alkylation unit 106 may include one or more processing units including, for example, a reactor 110 providing one or more reaction zones. Hydrocarbon feeds 7 may include supplies and/or mixtures of two or more components (e.g., a first hydrocarbon feed, a second hydrocarbon feed, through an nth hydrocarbon feed). For example, the one or more feeds 7 may include a first hydrocarbon feed including at least one olefin-containing component (e.g., an olefin feed 101a which may include butylenes, pentylenes, propylenes, or amylenes) and a second hydrocarbon feed having at least one paraffin-containing component (e.g., a paraffin feed 101b including, for example, an isobutane-containing component and/or an isopentane-containing component). It is contemplated that the olefin feed 101a may include non-olefin materials as well as olefins, and the paraffin feed 101b may include non-paraffin materials as well as paraffins.

Prior to entering a reaction zone of reactor 110, the olefin and paraffin feeds 101a and 101b may be subjected to drying and/or filtration processes to remove water, sulfur, and/or other contaminants. The olefin and paraffin feeds 101a and 101b may be mixed with a paraffin recycle stream 172b including material produced downstream in the alkylation process 12. In some embodiments, the mixture including the olefin feeds 101a, the paraffin feed 101b, and the paraffin recycle stream 172b may be supplied to the reactor 110, where it may be dispersed into an incoming stream including one or more catalysts 101c.

The reactor 110 of the alkylation unit 106 may include a vessel subdivided into the one or more reaction zones for processing the feeds under alkylation conditions, for example, as described herein. In some embodiments, the reactor 110 may also include a riser or section of piping within which reactions may occur. The reactor 110 may receive the one or more hydrocarbon feedstocks (e.g., an olefin feed 101a and paraffin feed 101b) and one or more catalyst feeds 101c to produce, as products, one or more corresponding unit materials. The one or more corresponding unit materials may include one or more of intermediate materials or unit product materials (e.g., collectively, reactor effluent 112 shown in FIG. 2A).

During processing in the reactor 110 of the alkylation unit 106, target process parameters may be controlled to maintain them within predetermined process parameter ranges. Such process parameters may include, for example, process pressure, and/or process temperature. In some embodiments, if process temperatures are too low, viscosity changes in the reactor 110 may inhibit the mixing of the olefins in the olefin feed and the one or more catalysts. Conversely, if process temperatures are too high, additional compounds may be formed in the reactor 110, and overall alkylate quality and/or the amount of high-octane materials produced may be relatively reduced.

Process control variables may include, for example, properties associated with the unit materials and/or processing units, such as feed content, catalyst strength (e.g., acid catalyst strength), water content, and/or other variables that may affect process control. Traditional methods for determining such parameters may be time-consuming, labor intensive, indirect, and/or inaccurate, for example, when used to correlate properties associated with the feeds, catalyst, and/or unit materials (e.g., specific gravity, vapor pressure, and/or other properties). For example, laboratory analysis may be used, but may involve extracting multiple samples from an alkylation process stream, which may provide handling and/or environmental drawbacks. Additionally, some methods may not sufficiently responsive to facilitate parameter adjustments that are responsive to short-term variations in the properties of some materials involved in the alkylation process, which may result in adversely affecting the alkylation operation. In at least some embodiments consistent with those described herein, analysis and/or determination of process-related parameters and/or material properties may result in comparatively more responsive and/or more accurate control of alkylation processes. For example, some embodiments may use measurement devices and/or assemblies, such as, for example, spectroscopic analyzers to maintain substantially continuous control of processes related to the alkylation processes. In some embodiments, process models and/or statistical techniques, such as multivariate analysis, may be employed, rendering process control that may be comparatively more rigorous and/or more consistent.

The input feedstocks may be analyzed for olefin content, paraffins, and isoparaffins in the C3-C5 range. Other hydrocarbon feedstock sample properties and/or unit material sample properties are also contemplated. The hydrocarbon feedstock may include a first hydrocarbon stream provided to an alkylation unit, for example, reactor 110. In some embodiments, one or more measurement devices 104a may be used to collect spectral data indicative of one or more properties and/or parameters associated with the hydrocarbon feedstocks. For example, the composition and purity of the olefin and isobutane feeds 101a and 101b to the reactor 110 may be determined to predict yield content and quality. In some embodiments, feedstocks 101a and 101b may have a content ratio indicative of relative amounts of one or more hydrocarbon classes present in the feedstocks. The content ratio may be reflected in one or more feedstock sample properties, intermediate material sample properties, and/or unit material sample properties sampled and measured by measurement devices, such as spectroscopic analyzers, before, during, and/or after the alkylation process(es) 12.

In some examples, the one or more properties and/or parameters of the input feedstocks may be correlated to traditional laboratory tests (e.g., tests performed via one or more primary test methods), including, for example, HPLC Heavy Distillate Analyzer (HDA) results for aromatic core type (e.g., 1-ring core, 2-ring core, 3-ring core, 4-ring core, and/or polars), and/or ASTM D2887 high temperature simulated distillation. The one or more properties and/or parameters may include, for example, elemental content, temperature, pressure, flow rate, API gravity, UOP K factor, distillation points, carbon residue content, nitrogen content, sulfur content, saturates content, thiophene content, single-ring aromatics content, dual-ring aromatics content, triple-ring aromatics content, quad-ring aromatics content, polynuclear aromatics content, benzene content, oxygenate percentage, ethanol content, flash, and/or viscosity. Other properties and/or parameters are contemplated.

In some embodiments, a measurement device 174b may be used to determine the composition and purity of the unit materials in paraffin recycle stream 172b to the reactor 110, for example, so that an amount of paraffins required in feed 101b may be accurately determined for the parameters of the alkylation process(es) 12. Measurement devices 104a and 174b may include spectroscopic analyzers positioned along one or more respective paths of the streams. In some examples, the predicted properties and/or parameters of the streams may be compared with target properties and/or parameters determined for the alkylation process(es) 12 and/or with those prescribed by an analytical model of the alkylation process(es) 12.

In some embodiments, the olefin feed 101a and/or the paraffin feed 101b may be dried and filtered in one or more processes to remove water and/or other materials before or after being combined with the paraffin recycle stream 172b. For example, the olefin and paraffin feeds 101a and 101b may be treated in a coalescer to remove water, sulfur, and other contaminants. Alternatively, or in addition, the olefin and paraffin feeds 101a and 101b may be treated with a mol sieve bed or another type of adsorbent to remove water. In some examples, the feed mixture (101a, 101b, and 101c) may be chilled (e.g., with refinery cooling water). The mixture may be fed to the reactor 110 of the alkylation process(es) 12 (FIG. 2A) and injected into a series of reaction chambers, where it may be dispersed into an incoming feed of catalyst 101c. The catalyst feed 101c may, in some examples, be pumped through the reactor 110 to mix with the reactants (e.g., the olefin and paraffin feeds 101a and 101b).

Due to the presence of the catalyst, the paraffins (e.g., isobutane) may react with the olefins to produce alkylate and/or other unit material products in the reactor effluent 112. The alkylate may include, for example, a seven- or eight-branched (C7 or C8 saturated carbon molecule) naphtha-range product. Other branched molecules may also be present in the reactor effluent 112, for example, depending on the content of the olefin feed used in the alkylation process 12. For example, nC4 and nC5 content in the olefin feed may pass through the reactor 110 without reacting. C3 olefins may generate C7 alkylate, C4 olefins may generate C8 alkylate, and C5 olefins may generate C9 alkylate. C6 and/or C10+ content in the olefin feed may generate acid soluble oil. The rate of deactivation of these components in the reactor 110 may vary depending on process parameters (e.g., the quality and/or feed rates of the olefin and paraffin feeds). In some embodiments, at least a portion of the catalyst and/or at least a portion of the paraffins (e.g., the isobutane) may be recovered and recycled through catalyst recycle feed 132a and paraffin recycle stream 172b, respectively.

In some embodiments, alkylation reactions may be catalyzed via one or more acids, and acid strength and/or acid purity may have an effect on the octane number of the alkylate produced. For example, aluminum chloride, sulfuric acid, hydrofluoric acid, and/or a zeolite catalyst may be used for the catalyst 101c. In some embodiments, a co-catalyst may be used, for example, to improve the rate, selectivity, and/or efficiency of the alkylation reaction.

Although the product yields and/or quality may be essentially the same for either a sulfuric acid or hydrofluoric acid catalyst, there may be differences in process operation and parameters due, at least in part, to differences in catalyst characteristics. For example, sulfuric acid may be a liquid at unit processing conditions (e.g., at processing temperature and/or pressure), while hydrofluoric acid may be a gas or a liquid at unit processing conditions. In some embodiments, hydrofluoric acid may be more readily separated and recovered from the resulting reactor effluent 112 than sulfuric acid, and less acid may be consumed as waste during the alkylation process(es) 12. The reactions may be exothermic, and higher molecular weight compounds may be formed from the olefins and paraffins. In some embodiments, heat created by the reactions may be removed from the reactor 110 via a heat exchanger (e.g., via coolant, such as plant cooling water and/or refrigeration) in order to control the reactions, to achieve relatively higher yields, and/or to achieve relatively higher alkylate quality from the alkylation process(es) 12.

The acid catalyst feedstock 101c may be supplied to the reactor 110 during the course of the alkylation process(es) 12 for reaction with the olefin and paraffin feeds 101a and 101b. The supply of the catalyst may include unprocessed acid make-up from a pure supply or a combination of unprocessed acid and used acid recycled back to the reactor 110. As shown in FIG. 2, in some embodiments, previously unprocessed catalyst 8 may be supplied (e.g., with a pump 107) and combined with a catalyst recycle feed 132a to form the catalyst feed 101c. One or more properties and/or parameters associated with the catalyst feed 101c may be determined, for example, via one or more measurement devices 104b, 124a, and/or 134a. Measurement devices 104b, 124a, and 134a may include spectroscopic analyzers positioned to measure and predict the properties and purities of the recycle streams (e.g., 122a and 132a) before and/or after settling and purifying processes, respectively. The measuring may be, for example, to monitor and maintain catalyst strength and concentration in the reactor 110, for example, by determining the amount of unprocessed catalyst 8 that is required, the weight percent of water in the recycle streams 122a and 132a, and/or the weight percent of acid oil in the recycle streams 122a and/or 132a before and after settling and purifying processes, respectively. Measurement devices, for example, spectroscopic analyzers 124a and 134a, may also be used to give an indication quantity and/or rate at which the catalyst is being consumed in the reactor 110 during the alkylation process 12.

The conversion of the reactants to the high-quality alkylate in the reactor 110 may occur relatively quickly, producing the alkylate compounds that form at least a part of an effluent mixture 112 and heat. As disclosed herein, heat from the reactor 110 may be dissipated by numerous methods. For example, plant cooling water and/or process steam may be directed through one or more heat exchangers. In other examples, at least some of the paraffin feed 101b may be vaporized and fed through a compressor to provide cooling.

Following the processing via the reactor 110, the alkylation process 12 may involve separating the resulting reacted products into a plurality of product streams. The intermediate products of the reactor effluent mixture 112 may be supplied to a series of other processing units in the alkylation unit 106. These processing units may provide secondary functions within the alkylation unit 106, and may include separators, acid strippers, and fractionating/distillation units. For example, as shown, the effluent mixture 112 may flow to a settler 120 to separate the intermediate products in the stream. The settler 120 may separate the components of the effluent mixture into multiple intermediate product streams 122a and 122b. For example, the settler 120 may divide the effluent mixture 112 into one or more intermediate streams 122a and 122b by stripping reacted acid catalyst in spent catalyst product stream 122a. The intermediate stream 122b from the settler 120 may include relatively high octane products, which may be thereafter blended into the gasoline pool and/or used by downstream processing units to create other finished products. For example, intermediate hydrocarbon streams 122b may include propane, n-butane (C₄H₁₀), alkylate, and/or other constituents. In some embodiments, the contents of stream 122b may be monitored in real-time by one or more measurement devices, such as a spectroscopic analyzer 124b, to provide data for control of the alkylation process(es) 12.

The spent catalyst stream 122a may include reacted catalyst stripped via the settler 120 from effluent mixture 112, which may be substantially a bottom phase, which may be supplied to (e.g., which may flow via gravity through) an acid regenerator 130. The composition of the spent catalyst stream 122a may depend, at least in part, on the composition of the olefin and paraffin feeds 101a and 101b, the alkylation reaction conditions, and/or process parameters associated with operation of the reactor 110. The spent catalyst stream 122a may include a plurality of components, including, for example, acid, water, and acid-soluble hydrocarbons in the form of acid oils (sometimes referred to as “red oil,” “polymeric oil,” “acid-soluble sludge,” and/or “conjunct polymer”), which may include organic contaminants. In some embodiments, these and other contaminants may be removed and/or separated from the alkylation process, for example, via a stream 132b.

The constituents of spent catalyst stream 122a may vary over a wide range during a commercial refining operation. As shown in FIG. 2A, in some embodiments, the composition and/or properties of spent catalyst stream 122a may be measured and/or predicted using the spectroscopic analyzer 124a to enhance control of the alkylation process(es) 12 through on-line measurement of the acid condition coming from the reactor 110. Using the measured composition, some embodiments of the alkylation process(es) 12 may operate under conditions in which the used or processed catalyst discharge concentration is above a minimum threshold, or is within a certain target range by weight percent of the spent catalyst stream 122a. For example, in response to predicted properties indicating reduced concentration, unprocessed catalyst 8 may be supplied to the catalyst feed 101c mixture. Alternatively, or in addition, the feed rate of the olefin feed 101a may be cut or decreased.

Acid oils and other contaminants may be removed from the spent catalyst stream 122a via the acid regenerator 130 and removed in stream 132b. The properties and/or the purity of the catalyst recycle stream 132a may be subsequently measured by measurement device 134a and, at least in part, be used to determine the quantity of unprocessed catalyst 8 needed to produce the feedstock of the catalyst feed 101c in sufficient concentrations for the reactor 110. In some embodiments, the catalyst recycle stream 132a may be replenished with an unprocessed catalyst 8 make-up “charge” of a higher purity from a catalyst supply. The catalyst feed 101c may be monitored by one or more measurement devices, such as spectroscopic analyzer 104b, positioned to analyze the catalyst feed 101c stream supplied to the reactor 110.

In some circumstances, a relatively large amount of a spent catalyst may be disposed as waste in order to remove a relatively small amount of acid oils and contaminants from the system. Thus, rapid, on-line, and accurate measurement of catalyst strength using measurement devices such as spectroscopic analyzers may be used to increase the efficiency of handling of acid waste from the catalyst and reduce the amount of unprocessed catalyst 8 required. For example, the acid recycle product in the catalyst recycle process may be controlled by determining the unit materials in the spent catalyst stream 122a and catalyst recycle stream 132a, which may include at least one of a concentration of the catalyst recycle product, a known weight percent of water in the catalyst recycle product, a known weight percent of acid oil in the catalyst recycle product, an amount of acid-soluble hydrocarbons in the catalyst recycle product, and/or an amount of organic contaminants in the catalyst recycle product. According to some embodiments, methods disclosed herein for monitoring of catalyst conditions via spectroscopic analyzers may facilitate operation of the alkylation process(es) 12 relatively closer to a minimized catalyst concentration threshold, for example, to reduce catalyst consumption and/or waste.

For example, a control system and/or operators may use data from measurement devices 104b, 124a, and/or 134a to select process parameters for the alkylation process(es) 12 to maintain catalyst concentration levels within a certain range of predetermined thresholds for the respective streams. For example, the control system may adjust process parameters and/or may continuously maintain catalyst concentration (e.g., acidity) above a designated minimum threshold of 90 weight percent in spent catalyst stream 122a. Lower or higher target concentrations may also be contemplated and targeted by adjusting the process parameters of the alkylation process(es) 12, as described herein. In some embodiments, the control system may select process parameters for the alkylation process(es) 12 to target and maintain a concentration level that is above, but held close to, the minimum threshold, for example, to reduce corrosion and/or extend the service life of process machinery in the alkylation unit 106. For example, a control system may use data from the spectroscopic analyzer 124a to operate at catalyst concentrations ranging from about 85 weight percent to about 97 weight percent (e.g., from about 91 to about 93 weight percent) in spent catalyst stream 122a above a designated minimum threshold (e.g., about 90 weight percent).

In some embodiments, consumption of the catalyst may be optimized, for example, due at least in part to the toxicity, volatility, and/or burdensome regeneration requirements. Recycled catalyst from the acid regenerator 130 may be returned in the rerun process to the reactor 110 in the recycle catalyst stream 132a, and an amount or range of contaminants and acid oils in the stream 132b may be used or targeted as measurement of process efficiency. For example, a control system and/or operators may manage one or more control variables to select process parameters for the alkylation process(es) 12, which may substantially maintain a catalyst consumption of, for example, about 0.5 pounds of catalyst consumed or wasted per gallon of produced alkylate. Alternatively, or in addition, a control system and/or operators may select process parameters to substantially maintain a catalyst consumption rate ranging from about 0.5 pounds to about 1.5 of catalyst consumed or wasted per gallon of alkylate produced.

The products in the intermediate stream 122b may flow from the settler 120 to one or more intermediate processing units of the alkylation process(es) 12 downstream from the reactor 110. In some embodiments, one or more intermediate processing units of the alkylation process(es) 12 may have columns that operate at successively lower pressures. This may allow the different unit materials and product streams to flow from unit to unit using the resulting pressure gradients, thus reducing the need for pumps and other equipment.

As shown in FIG. 2A, in some embodiments, at least some of the unit materials in the intermediate stream 122b may be directed to an isostripper unit 140. The isostripper 140 may include a tower, which may further separate the intermediate stream 122b into an isostripper bottoms product stream 142a and an isostripper overhead product stream 142b. The bottoms product stream 142a may include high-quality blending components, which may become a feed for a debutanizer unit 160. In some embodiments, the overhead product stream 142b may include propane, isobutane, and/or other components that may be used as a feedstock for a depropanizer unit 170. Measurement devices, such as spectroscopic analyzers 144a and/or 144b, may be provided to determine one or more properties of the isostripper bottoms product stream 142a and the isostripper overhead stream 142b, respectively.

In some embodiments, the debutanizer unit 160 may be configured to receive unit materials in a feedstock and produce two or more final products. For example, the debutanizer 160 may use fractional distillation to separate butane isomers from higher-boiling hydrocarbons in the stream. In some embodiments, the debutanizer 160 may be configured to produce normal butane (n-butane) as unit materials in a product stream 162a. In some embodiments, the product stream 162a may include an ethane fraction and/or a methane fraction. The properties and purity of the n-butane product in product stream 162a may be predicted with a measurement device, such as spectroscopic analyzer 164a. In some embodiments, an n-butane product in product stream 162a may be incorporated into further isomerization processing in the refining operation 10, for example, to produce additional isobutane for the paraffin feed of the alkylation process(es) 12. A relatively high-octane alkylate product 162b may also be produced by the debutanizer 160. Following treatment in the debutanizer 160, the alkylate product 162b may be routed to gasoline blending operations and/or may be subjected to additional solvent refinery processing. The properties of the alkylate product 162b may be determined to at least partially determine quantities of the alkylate product 162b to be used in blending operations. In some examples, at least a portion of the alkylate product 162b may be returned to the alkylation process(es) 12, for example, to produce aviation-grade blending stock. The properties of the alkylate product 162b may vary depending on the olefin feed used in the alkylation process(es) 12 and may be analyzed and predicted with a measurement device, such as spectroscopic analyzer 164b, for example, to determine suitability for use in various gasoline blending recipes.

In some embodiments, a depropanizer unit 170 may be provided and configured to receive unit materials from the isostripper overhead stream 142b from the isostripper 140. The depropanizer unit 170 may use fractionation to separate the isostripper overhead stream 142b into additional unit materials in two or more product streams. For example, a propane product 172a may be separated through fractionation, and other products added to the paraffin recycle stream 172b. The paraffin recycle stream 172b, which may include at least a portion of isobutane which has not been reacted/converted, may be routed back to the reactor 110 of the alkylation unit 106 of alkylation process(es) 12. Propane may normally be in a gaseous state, and in some embodiments, the tower of the depropanizer 170 may be maintained at a relatively high pressure to ensure a liquid phase for distillation. The propane product 172a may, in some examples, include other lighter products and may be used as a petrochemical feedstock and/or as a refinery fuel gas. In some embodiments, a defluorination catalyst may be used to remove organic fluorides from the propane product 172a.

The properties and/or contents of the propane product 172a may be measured and predicted by a measurement device, such as spectroscopic analyzer 174a. Properties of the paraffin recycle stream 172b may be analyzed with a measurement device, such as spectroscopic analyzer 174b, and one or more of the properties may be used, at least in part, to estimate or determine the quantity of higher-quality paraffin feed 101b needed for alkylation conditions in the reaction zone of the reactor 110.

FIG. 2B represents an example alkylation process 12′ during which sulfuric acid may be used as a catalyst for the reactions. For example, one or more hydrocarbon feeds 7′ may be supplied via a pump 105′ to a reactor 110′ of the alkylation unit 106′. The one or more hydrocarbon feeds 7′ may include at least one olefin-containing component (e.g., an olefin feed 101a′) and a second hydrocarbon feed including at least one paraffin-containing component (e.g., a paraffin feed 101b′). The olefin feed 101a′ may include non-olefin materials as well as olefins, and the paraffin feed 101b′ may include non-paraffin materials as well as paraffins.

Prior to entering the reactor 110′, the olefin and paraffin feeds 101a′ and 101b′ may be dried and mixed with a paraffin recycle stream 172b′ including material produced downstream in the alkylation process 12′. In some embodiments, the mixture including the olefin and paraffin feeds (101a′ and 101b′) and the paraffin recycle stream 172b′ may be dispersed into an incoming stream including one or more catalysts 101c′. During processing in the reactor 110′ of the alkylation unit 106′, target process parameters may be maintained within predetermined process parameter ranges.

Following the reaction in the reactor 110′, the reactor effluent 112′ may flow to an acid settler 120′ and used catalyst may be withdrawn in the recycle stream 122a′ and directed to an aftersettler 126 to be returned to the reactor 110′ in the catalyst recycle stream 132a′. An intermediate stream 122b′ may flow into the suction trap side of a suction trap flash drum 136, and the liquid and vapor portions of intermediate stream 122b′ may be separated. The suction trap/flash drum may include a two-compartment vessel having a common vapor space where refrigerant from a refrigeration loop 137 is accumulated in one compartment. At least a portion of condensed refrigerant from the refrigeration loop 137 may be collected in an accumulator 138 where it can be further treated by downstream processing units (e.g., depropanizer 170′).

In some embodiments, an intermediate stream 143 from the suction trap flash drum 136 in FIG. 2B may be subjected to fractionation processes in downstream processing units including, for example, one or more of a deisobutanizer 150, debutanizer 160′, depropanizer 170′, or other processing units. A paraffin recycle stream 172b′, which may include at least a portion of isobutane that has not been reacted/converted, may be recovered from at least one of the processing units and routed back to the reactor 110′. Measurement devices (e.g., spectroscopic analyzers 104a′, 104b′, 114′, 124a′, 124b′, 134a′, 141, 145, 154, 164a′, 164b′, 174a′, 174b′, and/or 175) may positioned along one or more respective paths of the streams of the alkylation process(es) 12 and/or 12′. In some embodiments, the predicted properties and/or parameters of the streams and operating parameters of the processing units may be compared with target properties and/or parameters determined for the alkylation process(es) 12 and/or 12′, and/or with those prescribed by an analytical model of the alkylation process(es) 12 and/or 12′.

The composition, distribution, and/or use of feedstocks and products may vary based on, for example, the capabilities, the layout, and/or the desired final products of different refineries. For example, the one or more upstream or downstream processing units or processes from the alkylation process(es) 12 and/or 12′ as described herein (and shown, e.g., in FIG. 2A and FIG. 2B) may be provided by way of example, and the systems and methods of this disclosure are thus not meant to be construed as limited to refineries and/or refining processes with those processing units or processes. For example, in some embodiments, the refining operation 10 may not have reforming capabilities and/or a saturated gas unit, and/or many light products may be transported or sold for further use or processing off-site, for example, rather than being used in alkylation process(es). In some embodiments, the capabilities, control, and/or quality of equipment used to supply feeds to the alkylation process(es) 12 (e.g., input feedstocks 28a, 28b, and/or 28c, as shown in FIG. 1) and carry out the alkylation process(es) (e.g., the reactors, isostripper, deisobutanizer, debutanizer, depropanizer, and/or other processing units shown in FIG. 2A and FIG. 2B) may result in products and/or fractions that are not completely separated from one another in the unit materials, resulting in product overlap and/or the need for further processing that is not detailed in this disclosure. For example, complete separation of products may not be required for the ordinary use of some products, but others, such as solvents for particular purposes (e.g., hexane, heptane, and/or aromatics), have uses that may require substantially pure compounds.

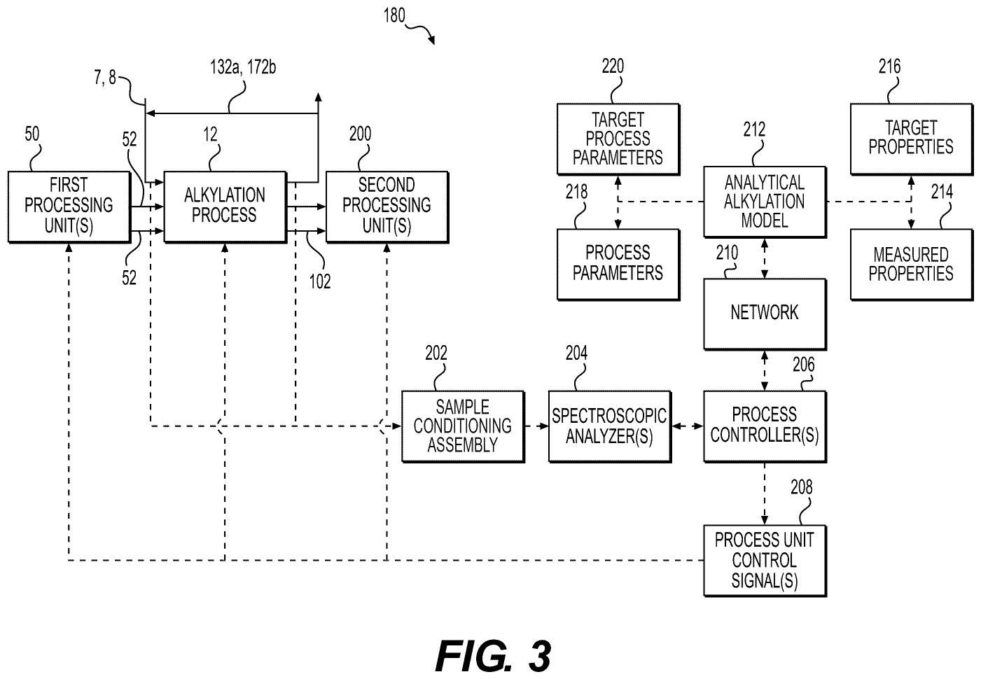

In some embodiments, other measurement devices, for example, in addition to the spectroscopic analyzers (or instead of spectroscopic analyzers), may be used for sensing process parameters, such as temperatures, pressures, and/or flow rates, and may be provided for the various feeds and products of the alkylation process(es) 12 for the purpose of controlling and enhancing the process(es). For example, temperature sensors may be used to sense temperatures of the various streams and convert the temperatures into electrical signals indicative of temperature for use by one or more process controllers 206 (see, e.g., FIG. 3). Such temperature signals may be communicated to the one or more process controllers 206, for example, via known wired communication protocols and/or known wireless communication protocols. In some embodiments, sensors configured to generate electrical signals indicative of pressure, flow rate, and/or other parameters may be provided, and those signals may be communicated to one or more of the process controllers, for example, via known wired communication protocols and/or known wireless communication protocols.

Different process parameters may be associated with different types of processing units. For example, the example isostripper unit 140 (FIG. 2A) may have process parameters associated with the feed (e.g., intermediate stream 122b), such as feed temperature, average feed rate, and/or acid entrainment and acid soluble oils in the feed line, which may be monitored and/or controlled for corrosion monitoring and acid balance in the processing unit. In some embodiments, the content of isobutane in the product streams (e.g., streams 142a and/or 142b) and/or the content of propane in the paraffin recycle stream 172b may be determined (e.g., intermittently and/or continuously), and operation of the isostripper unit 140 may be controlled based at least in part on one or more of such example parameters, for example, to enhance efficiency of operation of the isostripper unit 140. In some embodiments, a temperature profile in the tower of the isostripper unit 140 (e.g., bottoms temperature, normal isobutane draw temperature, feed tray temperature, intermediate/middle tray temperature(s), overhead temperature, dew point temperature(s), and/or other temperatures) may be determined (intermittently and/or continuously), and the temperature profile may be controlled, for example, for corrosion monitoring and flooding/operational constraints of the isostripper unit 140.

The locations and/or uses of spectroscopic analyzers, sensors, and other measurement devices and controllers disclosed herein are provided only as examples, and many other locations and/or uses are contemplated. For example, referring to FIG. 2A, measurement devices, such as the spectroscopic analyzers schematically represented at 104a, 104b, 114, 124a, 124b, 134a, 144a, 144b, 164a, 164b, 174a, and/or 174b should not be construed as limited to the approximate locations schematically depicted in FIG. 2A, and their uses should not be construed as limited to monitoring and predicting specific examples of the product properties and process parameters described herein. A lesser or greater number of spectroscopic analyzers may be used, and factors, such as end product needs and/or refinery-specific layouts and capabilities may be determinative of the application of spectroscopic analyzers and other measurement devices for controlling and/or enhancing control of certain refining processes.

In addition to those described, or alternatively, in some embodiments, the systems and methods disclosed may also be used to control and/or optimize refining operations off-line, such as remotely in a laboratory setting. These resources may be used, for example, in situations where a sample handling or preparation procedure may not be supported by on-line operations.

A spectroscopic analyzer may include a portable/field type spectroscopic analyzer, a laboratory-type spectroscopic analyzer, and/or a process-type (continuous on-line, in-line) spectroscopic analyzer. Samples for the spectroscopic analyzers may include unit material samples or intermediate material samples extracted from streams that may flow substantially intermittently or continuously past a point where the measurements by the spectroscopic analyzers are systematically made. In some embodiments, a single spectroscopic analyzer may be used to alternate between two or more streams, such as a feed stream and a product stream of a processing unit.

Once sampled from a stream, a beam of electromagnetic radiation may be transmitted into the material sample, resulting in the spectroscopic analyzer measuring a spectral response representative of the chemical composition of the sample material, which may be used to predict (or determine) properties of the sample material, for example, via the use of modeling. The spectral response may include a spectrum related to the absorbance, transmission, transflectance, reflectance, attenuated total reflectance, or scattering intensity caused by the material sample over a range of wavelengths, wavenumbers, or frequencies of the electromagnetic radiation. Data resulting from the spectral response may be processed by a signal processing device by, for example, by taking the first order or higher order derivative and applying statistical techniques (e.g., a least-squares regression, a multiple linear regression (MLR), a partial least squares regression (PLS), a principal component regression (PCR), a Gauss-Jordan row reduction, a multiple linear regression, and/or other methods) to provide one or more output signals indicative of one or more properties of one or more materials included in the analyzed stream, such as, for example, the concentration of the particular specie.

In some embodiments, the spectroscopic analyzers may be calibrated to generate standardized spectral responses. The spectroscopic analyzers may be calibrated using calibration data derived from one or more of a set of known reference fuels, a library of known spectral data, and/or an analytical model of the alkylation process. For example, using the standardized spectral responses, the calibration of the one or more spectroscopic analyzers may be updated prior to, or periodically during, on-line operations. Example methods are provided for determining and using the standardized spectral responses in, for example, U.S. Pat. No. 11,702,600, titled “Methods and Assemblies for Determining and Using Standardized Spectral Responses for Calibration of Spectroscopic Analyzers,” incorporated herein by reference in its entirety. The methods and assemblies described in the above-referenced application may be used to calibrate or recalibrate a spectroscopic analyzer when the spectroscopic analyzer changes from a first state to a second state, for example, the second state being defined as a period of time after a change to the spectroscopic analyzer causing a need to calibrate the spectroscopic analyzer. In some embodiments, the recalibration may result in the spectroscopic analyzer outputting a standardized spectrum, for example, such that the spectroscopic analyzer outputs a corrected material spectrum for an analyzed material, including one or more of an absorption-corrected spectrum, a transmittance-corrected spectrum, a transflectance-corrected spectrum, a reflectance-corrected spectrum, a attenuated total reflectance-corrected spectrum, or an intensity-corrected spectrum, and defining the standardized spectrum.

FIG. 3 is a schematic block diagram of an example alkylation process 12 including an example alkylation process control assembly 180 for enhancing control of the alkylation process 12, according to embodiments of the disclosure. Referring to FIG. 3, in some embodiments, the alkylation unit process assembly 180 may be used to at least partially (e.g., semi-autonomously and/or fully autonomously) control the alkylation process 12 of the refining operation 10. The alkylation process 12 may have material inputs including one or more hydrocarbon feedstocks 7 and one or more supplies of an unprocessed catalyst 8. A paraffin recycle stream 172b and a catalyst recycle stream 132a may be combined with the one or more hydrocarbon feedstocks 7 and one or more supplies of unprocessed catalyst 8 make-up (see, e.g., FIG. 2A and FIG. 2B). In some embodiments, the alkylation process 12 may also receive input feedstocks 52 from one or more first processing units 50. Input feedstocks 52 may be combined with (e.g., at least partially mixed with) the hydrocarbon feedstocks 7, the unprocessed catalyst 8, the recycle stream 172a, and/or the catalyst recycle stream 132a. The first processing units 50 may include one or more of a distillation/fractionation unit, a gas plant unit, a reforming unit, a catalytic cracking unit, and/or other processing units, for example, as described herein. The alkylation process control assembly 180 may also include a sample conditioning assembly 202 configured to prepare material samples for analysis by one or more spectroscopic analyzer(s) 204 to provide material sample spectra during the alkylation process 12. The alkylation process 12 may further include one or more process controllers 206 in communication with the spectroscopic analyzers 204 to prescriptively control parameters and operations of at least some of the first processing units 50, processing units of the alkylation process 12, and/or downstream second processing unit(s) 200.

For example, one or more of processing units of an alkylation unit 106 as part of alkylation process 12 may include an isostripper 140, a deisobutanizer 150, a debutanizer 160, and a depropanizer 170, for example, as shown in FIG. 2A and FIG. 2B. The process controllers 206 may be, for example, alkylation process controllers that receive one or more signals from the spectroscopic analyzer(s) 204 to predict sample content and/or properties of different process streams (e.g., feedstocks, unit materials, intermediate materials, unit product materials, recycle streams, and/or other materials) to enable the prescriptively control of different aspects of the alkylation process 12, for example, as described herein. Based at least in part on the sample content and/or the sample properties, the process controllers 206 may be configured to alter one or more control variables, such as process parameters of the alkylation process 12, for example, in response to variance in the sample properties from predetermined target properties. For example, the prescriptive control may include controlling: one or more operating parameters associated with the depropanizer 170; the content of intermediate materials or unit product materials produced by the depropanizer 170; one or more operating parameters associated with the isostripper 140; the content of one or more unit materials produced by the isostripper 140; one or more operating parameters associated with the debutanizer 160; and/or the content of one or more unit materials produced by the debutanizer 160. The prescriptive control may further include causing the alkylation process 12 to produce unit materials that converge, for example, in an iterative fashion, on the predetermined target properties.

In some embodiments, the refining operation may have one or more second processing unit(s) 200 arranged in line with, or downstream of, the first processing units 50 and/or the alkylation process 12 to receive unit materials 102 (e.g., intermediate materials or unit product materials) as feedstocks from the first processing units 50 and/or the alkylation process 12. The one or more second processing units 200 may include, for example, one or more of a hydrotreater unit, a hydrogen recovery unit, a solvent processing unit, a thermal processing unit, an adsorption unit, or other processing units for processing the unit materials 102 from the first processing unit(s) and/or the processing units of the alkylation process 12 into final products and/or forms appropriate for supply to a third party and/or use by other downstream processing units.

As schematically depicted in FIG. 3, solid lines indicate material feed flows and product flows, which may be used to supply one or more unit materials, which may include one or more of intermediate materials and/or unit product materials, for analysis. Dashed lines indicate sample analysis, control signals, and control system flows of the alkylation process control assembly 180. In some embodiments, the alkylation process control assembly 180 may include one or more measurement devices (e.g., spectroscopic analyzers 204), which may be used to receive (e.g., on-line), analyze, and/or generate one or more spectra indicative of properties and/or contents of samples of a feedstock and/or product flow (e.g., the hydrocarbon feedstocks 7, the unprocessed catalyst 8, the recycle stream 172a, the catalyst recycle stream 134a, the input feedstocks 52 from the first processing units 50, the downstream unit materials 102, etc.) and/or indicative of properties of samples of one or more intermediate materials produced by one or more of the first processing unit(s) 50, the processing units of the alkylation process 12, the second processing unit(s) 200, and/or other processing units of the refining operation 10.

In some embodiments, one or more of the first processing units 50 may include, for example, a crude atmospheric distillation unit, a gas plant unit, a reformer unit, and/or an FCC unit upstream of the alkylation process 12. The second processing unit(s) 200 may include, for example, further fractionation units, strippers, regenerator components, hydrotreaters, and/or processing units downstream of the alkylation process 12.

In some embodiments, one or more of the spectroscopic analyzers 204 of the alkylation process control assembly 180 may be reinforced and/or hardened for use in an on-line analyzing process, and in some embodiments, one or more of the spectroscopic analyzers 204 may be at least partially housed in a temperature-controlled and/or explosion-resistant cabinet. In some embodiments, similar techniques to those described herein may be used for other types of sample analyses. For example, a photometer with present optical filters moving successively into position, may be used as a type of spectroscopic analyzer. In other embodiments, a fiber optic probe in communication with one or more of the spectroscopic analyzers 204 may be inserted directly into the conduit or conduits containing the stream to be sampled to facilitate analysis by one or more of the spectroscopic analyzers 204, which may prevent the need to extract the sample of the stream for analysis from a separate conduit or sample collection system.

Referring to FIG. 3, in some embodiments, the alkylation process control assembly 180 also may include one or more process controllers(s) 206 in communication with one or more of the spectroscopic analyzers 204 and configured to control one or more aspects of the alkylation process 12 and/or other refining processes. The control of the one or more process controllers(s) 206 may include controlling one or more operating parameters against operating constraints associated with one or more of the first processing unit(s) 50, processing units associated with the alkylation process 12, or the second processing unit(s) 200 in the refining process. In some embodiments, the alkylation process control assembly 180 may have separate process controllers for the alkylation process 12. The one or more process controller(s) 206 may be configured to predict (or determine) one or more unit material sample properties and/or parameters associated with the unit material samples based at least in part on the unit material sample spectra generated by the one or more spectroscopic analyzers 204. For example, as described herein, each of the one or more spectroscopic analyzer(s) 204 may be configured to output a signal or signals indicative of sample properties as control variables and communicate the signal(s) to the one or more process controller(s) 206, which may be configured to mathematically manipulate the signal (e.g., take a first order or higher order derivative of the signal(s) and apply statistical techniques, such as one or more of a partial least squares analysis, a principal component regression, a Gauss-Jordan Row reduction, or a multiple linear regression) received from the one or more spectroscopic analyzer(s) 204. In some embodiments, the one or more spectroscopic analyzer(s) 204 may be linked to a signal processing device to permit numerical treatment of the spectral range.

For example, after analyzing a unit material sample via a spectroscopic analyzer to produce a unit material sample spectra, the sample spectra may be manipulated to predict one or more unit material sample properties. The manipulated signal may be used to generate material properties and/or process parameters of interest, for example, as described herein. To facilitate control, the signal(s), material properties, and/or process parameters may be output to a display in communication with the one or more process controller(s) 206 and/or spectroscopic analyzer(s) 204. In some embodiments, analytical models simulating and/or characterizing the alkylation process 12 may be derived from signals obtained from known conditions or from spectroscopic analyzer measurement of the one or more unit materials (e.g., feedstocks and products of the alkylation process and associated processes).

For example, referring to FIG. 3 and the alkylation process 12, various process unit control signals 208 may be used to prescriptively control an alkylation unit (e.g., example processing units from FIG. 2A and FIG. 2B such as a reactor 110/110′, an isostripper 140, a deisobutanizer 150, a debutanizer 160/160′, a depropanizer 170/170′, and/or other processing units associated with the alkylation process) and/or associated processing units. For example, one or more process unit control signals 208 may be configured to control process parameters 218 associated with the supply of the hydrocarbon feedstock 7 and the input feedstocks 52 (e.g., one or more of the olefin-containing feed or the isobutane-containing feed) and the catalyst feed (e.g., such as a combination of the catalyst recycle stream 132a and unprocessed catalyst 8) to the reactor 110 of the alkylation process 12. The process parameters 218 may include, for example, temperatures, pressures, feed ratios, feed space velocities, purities, concentrations, and/or other parameters.

In some examples, the prescriptive control by the alkylation process control assembly 180 may be tailored, based at least in part, on a set of one or more desired process parameters 218 for the alkylation process 12, which may be configured, for example, to preserve and/or maintain refinery machinery, so as to reduce downtime for inspection and/or maintenance. Alternatively, or in addition, the prescriptive control may be tailored, based at least in part, on desired target contents or target properties 216 of the unit materials in the alkylation products, such as the quality of the produced alkylate and/or other gasoline blending characteristics. For example, the unit materials may have sample properties including one or more of a Research Octane Number (RON), a Motor Octane Number (MON), an Anti-Knock Index (AKI), a total aromatics content, a total butane content, an amount of isobutane, an amount of n-isobutane, a total olefins content, an amount of gasoline, an amount of propane, an amount of pentane, an amount of hydrogen sulfide, an amount of sulfur, a level of water content, an aniline point, an amount of propane, an amount of benzene, an amount of toluene, an amount of xylene, total Benzene Toluene Xylene (BTX) content, total Benzene Toluene Ethylbenzene Xylene (BTEX) content, a vapor pressure, distillation properties/parameters, a drivability index, a density, a specific gravity, an American Petroleum Institute (API) gravity, an amount of alcohol, or an Ethanol (EtOH) kick prediction. Other sample properties are also contemplated and may be targeted. In some embodiments, the prescriptive control may also be targeted to increase or maximize the yield of the alkylate and/or other unit product materials produced during the alkylation process 12.

In some embodiments, the spectroscopic analyzers may be configured to generate a measurement signal indicative of the one or more of the measured absorbance, the measured transmittance, the measured reflectance, the measured attenuated total reflectance, or the measured scattering intensity associated with a material sample from the sample stream. The measurement signal may be associated with one or more of individual wavelengths, individual wavenumbers, or one or more of a partial spectrum or a whole spectrum from the sample. The one or more process controller(s) 206 may be configured to receive the measurement signals and control, based at least in part on the measurement signals, one or more of: one or more properties, content, quality, or quantity, of one or more unit materials produced by the alkylation process 12. For example, the one or more process controller(s) 206 may be configured to compare differences between (a) one or more predicted properties or measured properties 214 of an alkylate product 162b and (b) one or more target properties or target properties 216 of the alkylate product, and to cause the alkylation process 12 to converge on an alkylation product having one or more properties within respective ranges of one or more preselected target properties and/or one or more contents within respective ranges of target contents 216. The preselected target properties and/or target contents 216 for the alkylate product 162b may be determined by, for example, an analytical alkylation model 212 characterizing and/or simulating the alkylation process 12.

In some embodiments, the analytical alkylation model 212 may include an analytical alkylation model in communication with (e.g., accessible by) the one or more process controller(s) 206. In some embodiments, the analytical alkylation model 212 may include a machine-learning model configured to be updated periodically, intermittently, and/or continuously, for example, during the course of refining operations (semi-autonomously and/or autonomously). The one or more process controller(s) 206 may be configured to determine differences between (a) the target parameters 220 and/or target contents or properties 216 and (b) the actual measured process parameters 218 and/or measured properties 214, for example, as measured by the spectroscopic analyzers 204. In some examples, multiple signals indicative of a plurality of measured parameters 214 and a plurality of process parameters 218 (e.g., a first signal, a second signal, a third signal, a fourth signal, . . . an nth signal) may be used, individually and/or in combination, as control variables by the analytical alkylation model 212. In some embodiments, the one or more process controller(s) 206 may be configured to determine one or more differences between (a) the target operating parameters 220 and (b) the actual measured operating parameters 218, and/or between (a) the target material properties 216 and (b) the actual measured material properties 214. The one or more controller(s) 206 may be configured to generate one or more control signals 208, based at least in part on the one or more differences, to adjust one or more of the set of unit operating process parameters 218, for example, to reduce or minimize the one or more differences. During a later, subsequent time period, a new set of one or more differences may be determined, such that the measured process parameters 218 and measured properties 214 may be controlled to converge (e.g., to iteratively converge) on the target operating parameters 220 and/or the target properties 216.

In some embodiments, the differences may be based at least in part on values or targets determined analytically or empirically. For example, one or more differences may be based on comparisons with material data in a known material database. Alternately or in addition, one or more differences may be determined via comparison of the process parameters 218 with one or more threshold values associated with operation of the one or more first processing units 50, processing units of the alkylation process 12, and/or the one or more second processing units 200. In some embodiments, at least some of the differences may be determined via comparison with predetermined target properties 216 (and/or target content and/or content ratios) derived, at least in part, from the analytical alkylation model 212 of the alkylation process 12. The target properties 216 may include, for example, target properties associated with one or more of the unit materials, intermediate materials, the unit product materials, the one or more catalyst(s), the one or more olefin feed sample properties, the one or more paraffin feed sample properties, or downstream material sample properties, for example, as described herein.

In some embodiments, the process parameters 218 for prescriptive control using the one or more process controllers 206 of the alkylation process control assembly 180 may include one or more of: (a) olefin feed parameters associated with the olefin feed having variables such as one or more of a rate of supply, a pressure, or a preheating temperature; (b) one or more paraffin feed parameters associated with the paraffin feed having one or more of a rate of supply, a pressure, or a preheating temperature; or (c) one or more catalyst feed parameters associated with the catalyst feed having one or more of a rate of supply, a pressure, or a temperature. Additional process parameters may include one or more of: (a) content of the olefin feed supplied to the reactor; (b) content of the paraffin feed supplied to the reactor; (c) a temperature inside the reactor; (d) a water content inside the reactor; (e) a purity of the paraffin feed supplied to the reactor; or (f) a ratio of the olefin feed to the paraffin feed supplied to the reactor.

The processing in the alkylation unit 106 may occur at designated setpoints or ranges of temperature and/or pressure. For example, the processing may occur at a temperature ranging from about 50 degrees Celsius (C) to about 280 degrees C. and/or at a pressure ranging from about 300 lbs. per square inch (psi) to about 1,000 psi, or at a temperature ranging from about 1 degree C. to about 40 degrees C. and at a pressure ranging from about 14 psi to about 150 psi. The controlling may include controlling a temperature inside the reactor 110 of the alkylation unit 106 to be equal to or less than 50 degrees C.

In some embodiments, one or more of the olefin feed or the paraffin feed may be composed of a blended hydrocarbon feed including a plurality of hydrocarbon feeds from respective hydrocarbon feed flows. The one or more process controllers 206 may be configured to control a ratio of the olefin feed to the paraffin feed supplied to the alkylation process, thereby to control an octane level or target composition associated with the alkylate product.

The alkylation process control assembly 180 may be configured execute or operate the analytical alkylation model 212, for example, to improve the performance of the alkylation process 12. The processing units associated with the alkylation process 12 may include, for example, a reactor 110 and one or more acid separators or settlers 120 receiving an effluent 112 from the reactor 110. In some embodiments, the analytical alkylation model 212 used in the prescriptive control may include a machine-learning trained model. The analytical alkylation model 212 may be configured to receive current alkylation process data on-line, for example, during the alkylation operation, and the analytical alkylation model 212 may be updated and/or refined based at least in part on the process data.

Using spectroscopic analyzers and/or other measurement devices to intermittently or continuously monitor the alkylation process 12, the analytical alkylation model 212 may be operated to prescriptively control at least a portion of the process, for example, such that one or more of the products produced has properties comparatively closer to optimum or desired product specifications. For example, the prescriptive control may include controlling one or more of: (a) one or more operating parameters associated with the reactor of the alkylation unit; the purity of the one or more catalysts supplied to the reactor of the alkylation unit; (b) the feed rate of the one or more catalysts supplied to the reactor of the alkylation unit; (c) a water content inside the reactor; (d) the content of the intermediate materials or unit product materials associated with the effluent produced by the reactor; (e) one or more operating parameters associated with the one or more acid settlers; or (f) the content of the intermediate materials or unit product materials produced by the one or more acid settlers.

In some embodiments, some portions of the analytical alkylation model 212 used in the prescriptive control may be hosted separately, such as on an independent computer system, server, or a network 210. In some embodiments, the full analytical alkylation model 212 used in the prescriptive control may be integrated and hosted commonly on one or more computer systems (not shown), for example, on the same network 210. Alternatively, at least a portion of the analytical alkylation model 212 may be represented by a common set of processor instructions stored on a memory device.

Aspects of the subject matter described herein may be practiced on, or in conjunction with, other computer system configurations beyond those described herein, including, for example, multiprocessor systems, microprocessor-based or programmable consumer electronics, minicomputers, mainframe computers, handheld computers, mobile telephone devices, tablet computing devices, special-purposed hardware devices, network appliances, and the like. Some or all of the computer system configurations may be connected to the network 210.

In some embodiments, other spectroscopic analyzers and/or other measurement devices may be used to analyze, for example, during the alkylation process, one or more downstream properties and/or parameters associated with a unit material stream produced by one or more of the second processing units 200 downstream of the alkylation process 12. One or more of the second processing units 200 may, for example, include at least a portion of a gasoline blending operation. The downstream properties and/or parameters may reflect characteristics and/or operational performance of the alkylation process 12. The downstream properties may be used as control variables, for example, in feedback control to prescriptively control the alkylation process 12.

For example, in some embodiments, the one or more process controllers 206 may be configured to receive a signal or signals indicative of the one or more downstream properties and/or parameters and determine, based at least in part on the alkylation process model 212, one or more differences between (a) a set of actual downstream operating parameters for the one or more second processing units 200 and (b) a set of target downstream operating parameters for the one or more second processing units. The one or more process controllers 206 may use the differences, at least in part, to adjust one or more of the set of actual downstream operating parameters to reduce and/or minimize the one or more differences. In some examples, the one or more process controllers 206 may adjust the target parameters 220 and/or the actual process parameters 218 of the upstream first processing units 50 and/or processing units of the alkylation process 12, for example, to reduce and/or minimize the one or more differences in the downstream operating parameters for the one or more second processing units 200.

In some embodiments, the one or more process controllers 206 may be in communication with the spectroscopic analyzers 204 and may execute at least a portion of the analytical alkylation model 212, for example, so as to update (e.g., iteratively update) the analytical alkylation model 212 based at least in part on improvements in the current properties and/or parameters of the alkylation process 12. In some embodiments, this may result causing the alkylation process 12 to achieve material outputs that more accurately and responsively converge on one or more of the target contents or target properties 216 and/or the target process parameters 220. In some embodiments, the prescriptive control may result in optimizing one or more target properties of the one or more unit materials, the one or more intermediate materials, and/or the one or more unit product materials produced during the alkylation process 12. In some embodiments, the prescriptive control may result in optimizing the operation of one or more of the first processing units 50 and/or the second processing units 200 to achieve material outputs that more accurately and responsively converge on one or more of the target properties or target process parameters. In some embodiments, additional analytical process models may be used to better reflect and update the target process parameters 220 and/or the target contents 216 associated with the analytical alkylation model 212.

In addition to, or instead of, an analytical alkylation model 212, one or more of the process controllers 206 may include a computer-assisted feedback control system that, based at least in part on predicted sample properties generated by the spectroscopic analyzers 204, alters one or more process parameters 218 of one or more processing units in response to a variance in the predicted sample properties from predetermined target properties 216. For example, the measured properties 214 associated with a specific feed from the spectroscopic analyzers 204 may be used to determine whether change are needed in unit process parameters 218 to make a product having specified target properties 216 in feed-forward control. In some embodiments, the measured properties 214 from the spectroscopic analyzers 204 associated with a specific product may be used to identify changes in one or more feeds or unit process parameters 218 that may result in a product having specified target properties in feedback control.

The specified target properties 216 may include a plurality of properties. The plurality of properties may include, for example, one or more of a Research Octane Number (RON), a Motor Octane Number (MON), an Anti-Knock Index (AKI), a total aromatics content, a total olefins content, a benzene content, a toluene content, a xylenes content, a total Benzene Toluene Xylene (BTX) content, a total Benzene Toluene Ethylbenzene Xylene (BTEX) content, a vapor pressure, distillation properties/parameters, a drivability index, a density, a specific gravity, an American Petroleum Institute (API) gravity, an alcohol content, or an Ethanol (EtOH) kick prediction.

Embodiments of the present disclosure may also be directed to a sample conditioning system configured to condition an intermittent sample stream and/or a continuous sample stream from the alkylation process to enhance the analysis of a spectroscopic analyzer. For an on-line spectroscopic analyzer, an intermittent sample stream or a continuous sample stream may be subjected to a process (e.g., conditioning) when preparing a representative sample for analysis by a spectroscopic analyzer. The conditioning process may be implemented using the sample conditioning system described herein, as well as components for fast loop and pretreatment operations. The function of these components may enhance the performance of a spectroscopic analyzer. For example, a sample substantially free of contaminates, without free water, and/or maintained within specified temperature tolerances may result in more accurate measurements by a spectroscopic analyzer.

In some embodiments, the alkylation process control assembly 180 may include a sample conditioning assembly 202, which may be positioned to condition one or more sample streams from the alkylation process 12. The one or more sample streams may include a plurality components from the first processing unit(s) 50, processing units of the alkylation process 100 (see, e.g., FIG. 2A and FIG. 2B), the downstream processing unit(s) 200, and/or a combination of streams from these sources, which may be conditioned prior to being supplied to the one or more spectroscopic analyzer(s) 204. For example, the various sample streams to be conditioned may include one or more of the olefin feed, the paraffin feed, streams of the catalyst recycle product (122a, 132a, and/or 101c), the paraffin recycle stream 172b, and/or the alkylate product 162b and/or other final products. In some embodiments, at least some of these process streams may be relatively complex mixtures of hydrocarbon types, which the analytical alkylation model 212 may be used to characterize, after conditioning and analysis by the spectroscopic analyzers 204. For example, the analytical alkylation model 212 may be configured to receive material properties measured by the spectroscopic analyzers, such as density, K factor, true boiling point (TBP), ASTM distillation curves, and/or other properties, and compare them to target values from the used by analytical alkylation model 212.

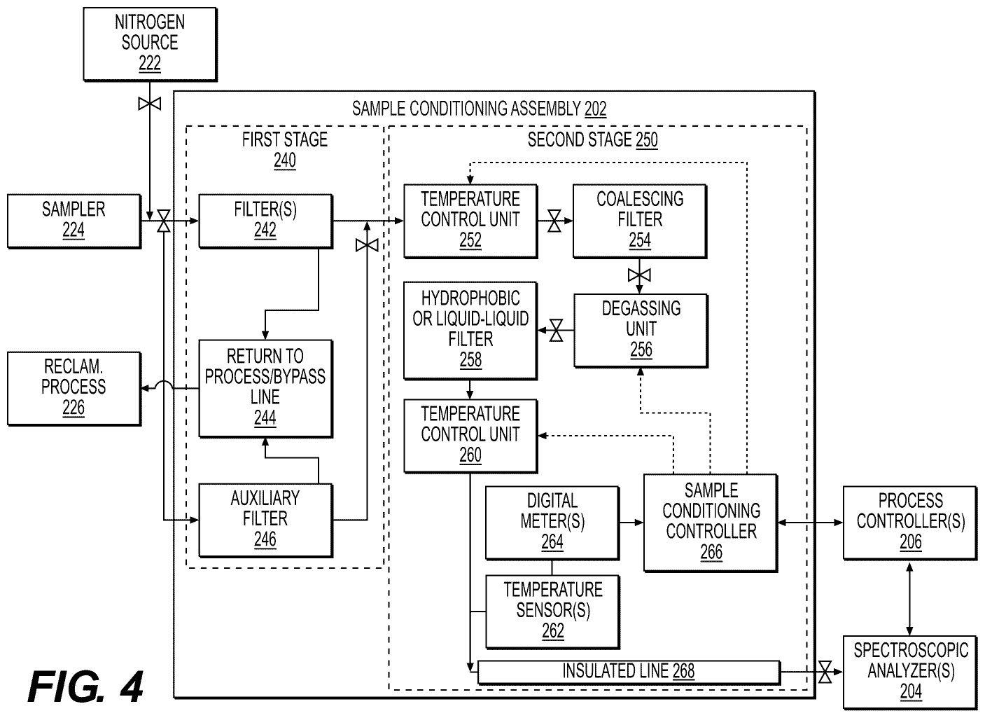

FIG. 4 is a schematic block diagram of an example sample conditioning assembly 202, according to embodiments of the disclosure. As illustrated in FIG. 4, the sample conditioning assembly 202 may include a sampler 224 configured to extract a material sample from the sample stream from a point or points associated with the alkylation process 12 (e.g., before, during, and/or after the alkylation process 12). The material sample may be in the form of an intermittent sample stream and/or a continuous sample stream. The sample conditioning assembly 202 may include a sampler 224, which may include a sample probe, a sample supply pump, and/or a varying header pressure from a header configured to provide a consistent flow of the sample stream to the sample conditioning assembly 202. The sample conditioning assembly 202 may include an enclosure for housing at least some of the components associated with the sample conditioning assembly 202. In some embodiments, the sample conditioning assembly 202 may include subsystems for performing processes such as, for example, water and contaminant filtration, temperature control, and/or degassing treatment. For example, in some embodiments, a vapor pressure of a desired alkylate product sample may be defined by a predetermined range (e.g., between about 5.5 and about 15 pounds per square inch (psi)). In some embodiments, a degasser may be provided, for example, for an alkylate product sample stream having a relatively higher vapor pressure than the predetermined range, to remove gases or bubbles from the sample stream.

In some embodiments, as shown in FIG. 4, the sample conditioning assembly 202 may include a first stage 240 and a second stage 250. The first stage 240 may include a first set of one or more filters 242 including a plurality of membranes configured to filter the extracted sample to thereby reduce the amount of particulates and water from the extracted sample. The first stage 240 may further include an auxiliary filter 246 in fluid communication with the sampler 224 and connected in parallel to the first set of one or more filters 242 for use during a maintenance of the first set of one or more filters 242. The auxiliary filter 246 may be configured to receive the extracted sample from the sampler 224. Whichever is in use, the first set of filters 242 or the auxiliary filter 246, may produce a filtered sample, which may include a substantially continuous sample stream. The removed particulates and water may be routed to a reclamation process 226, a sample recovery assembly, or a pump, for example, via a bypass line 244 configured to route the reduced particulate and water.

In some embodiments, at least a part of the sample conditioning assembly 202 may also be flushed out with a flow of compressed gas, such as nitrogen from a nitrogen source 222 and/or air to remove particulates in the sample conditioning assembly 202, thereby allowing for maintenance activities of the sample conditioning assembly 202. For example, when the sample is directed to the auxiliary filter 246, at least the first set of one or more filters 242 may be flushed and cleaned with the flow of compressed gas.

The second stage 250 of the sample conditioning assembly 202 may include a first temperature control unit 252. The first temperature control unit 252 may include one or more of a chiller, a heater, or other heat exchanging components in fluid communication with the first set of one or more filters 242 positioned to receive the filtered sample and change a temperature of the filtered sample to a predetermined temperature, which may render it more suitable for sample analyses. A sample having a regulated temperature may thus be supplied to the spectroscopic analyzer(s) 204, which may yield relatively more accurate analysis. The first temperature control unit 252 may heat or chill the sample to the predetermined temperature, for example, to provide a temperature-adjusted sample having a temperature within a first preselected temperature range. The second stage 250 may also include a degassing unit 256 configured to degas the filtered sample (e.g., a temperature-adjusted degassed sample).

The second stage 250 of the two-stage sample conditioning process may continue with a process for removing water or moisture from the sample via a second set of one or more filters. The second set of one or more filters may include a coalescing device, such as a coalescing filter 254, for example. The coalescing filter 254 may be configured to remove or reduce water and/or eliminate any liquid contaminants and/or mist based on one or more of three methods: (1) direct impact for particles having a particle size being approximately 1 micron or larger, (2) interception for particles having a particle size ranging from approximately 0.1 and 0.6 microns, and (3) diffusion for particles having a particle size approximately 0.1 micron or smaller. The water and/or moisture that has been removed by the coalescing filter 254 may be routed to the bypass line 244. In some embodiments, for example, the second set of filters in the second stage 250 may be configured to remove the remaining water and/or moisture from the sample, for example, using a hydrophobic filter 258 and/or a liquid-liquid membrane filter. The hydrophobic filter 258 may be configured to prevent passage of remaining water and/or moisture in the sample. The hydrophobic filter 258 may include a known hydrophobic filter, such as a hydrophobic polytetrafluoroethylene (PTFE) membrane filter. A hydrophobic PTFE membrane filters may include a thin, highly porous film that behaves as an absolute retentive membrane. In some embodiments, the second stage 250 of the two-stage conditioning assembly process may further have the coalescing filter 254 positioned after the hydrophobic filter 258 or liquid-liquid membrane filter.

In some embodiments, the second stage 250 of the sample conditioning assembly 202 may further include one or more thermometers or temperature sensors 262 in communication with one or both of the first temperature control unit 252 and the second temperature control unit 260, and configured to generate one or more temperature signals indicative of the temperature of one or more of the filtered sample, the temperature-adjusted sample, the degassed sample, or the temperature-adjusted degassed sample. The system may additionally include one or more digital meters 264 configured to monitor and/or display the measured temperature of the filtered sample. The second stage 250 may also include a second temperature control unit 260 in fluid communication with one or more of the degassing unit 256 or the second set of one or more filters and positioned to change a temperature of the degassed sample to provide a temperature-adjusted degassed sample, such that the temperature-adjusted degassed sample has a temperature within a second preselected temperature range for more accurate property analysis via the one or more spectroscopic analyzers 204.

The second stage of the sample conditioning assembly 202 may further include a sample introducer in fluid communication with the first temperature control unit 252 and the second temperature control unit 260 via an insulated sample line 268. The sample introducer may also be connected to the spectroscopic analyzer(s) 204 via the insulated sample line 268 to supply the conditioned sample to the spectroscopic analyzer(s) 204. In some embodiments, the first temperature control unit 252 (and the associated chillers, heaters, and/or other components) may also be connected to the sample introducer via an optical fiber cable configured to maintain the conditioned sample at a predetermined temperature before reaching the spectroscopic analyzer(s) 204. The predetermined temperature may be, for example, between 70° F. and 72° F. Other temperature ranges for the predetermined temperature are also be contemplated, depending on the design and/or type of the spectroscopic analyzer(s) 204.

Alternatively, or in addition, in some embodiments, one or more temperature correction factors may be applied to, for example, one or more of the sample spectra generated by the spectroscopic analyzer(s) 204. The correction factor(s) may be derived, for example, from literature, historical operational data, empirical data, one or more analytical models related to the refining process(es), and/or other sources. The sample spectra may include, for example, the paraffin feed sample spectra, the olefin feed sample, spectra, and/or the sample spectra of one or more unit materials produced during the alkylation process.

In some embodiments, the second stage 250 of the sample conditioning assembly 202 may further include a sample conditioning controller 266 in communication with the temperature sensors 262. The sample conditioning controller 266 may include a processor and a memory configured to store the measured temperature of the conditioned sample as temperature data in a data structure in the memory associated with the controller 266 (or, as described, one or more known temperature correction factors may be applied). The stored temperature data may be transmitted to a monitoring server via a cable or a network and may also be shared with one or more process controllers 206 associated with the alkylation process 12.

Following conditioning by the sample conditioning assembly 202, the spectroscopic analyzer(s) 204 may use the reflected signal from a beam to determine a spectral response representative of the chemical composition of a material sample, for example, as described herein. The one or more spectra of the conditioned samples may be generated at one or more wavenumbers or wavelength bands (wavenumber=1/wavelength). The generation of the spectra may include receiving a selected number of spectra from a conditioned sample. The generation of the spectra may also include determining an average spectrum based on the received selected number of spectra of the conditioned sample stream.

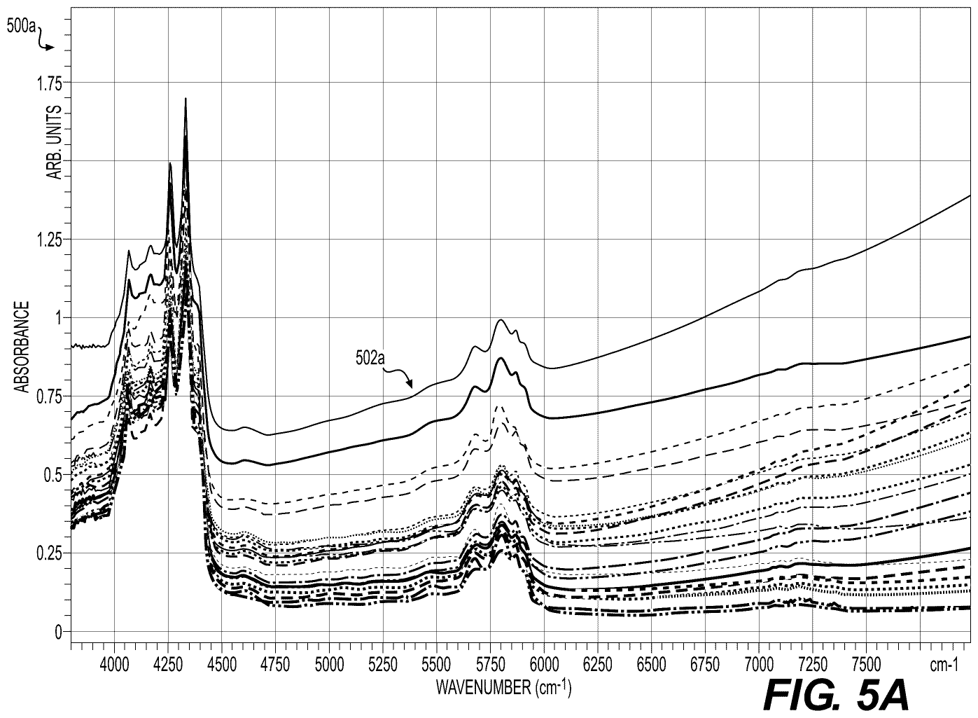

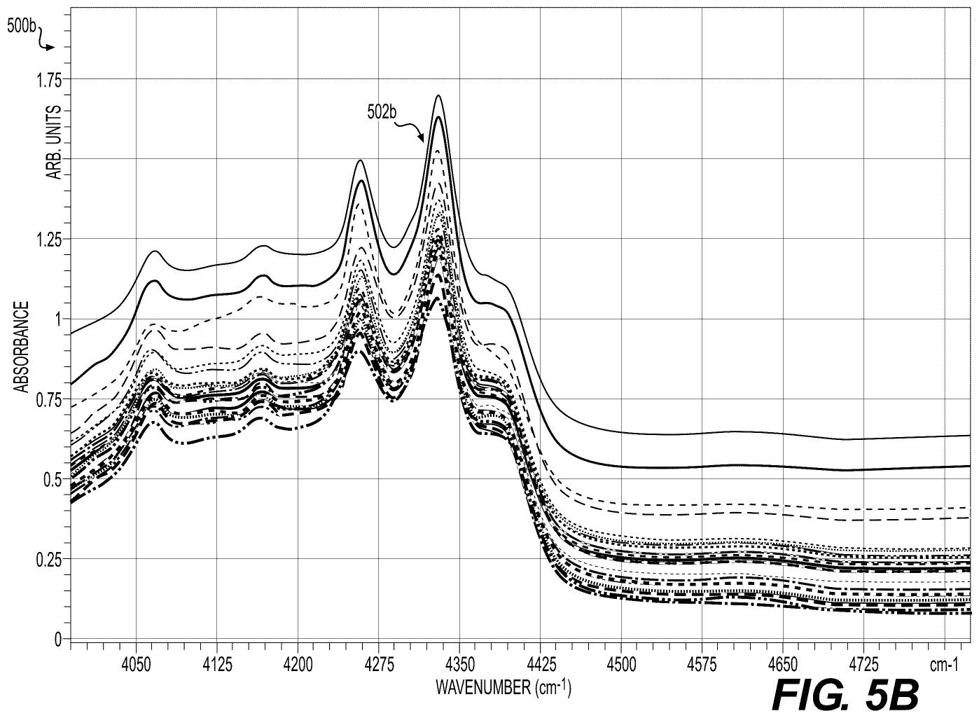

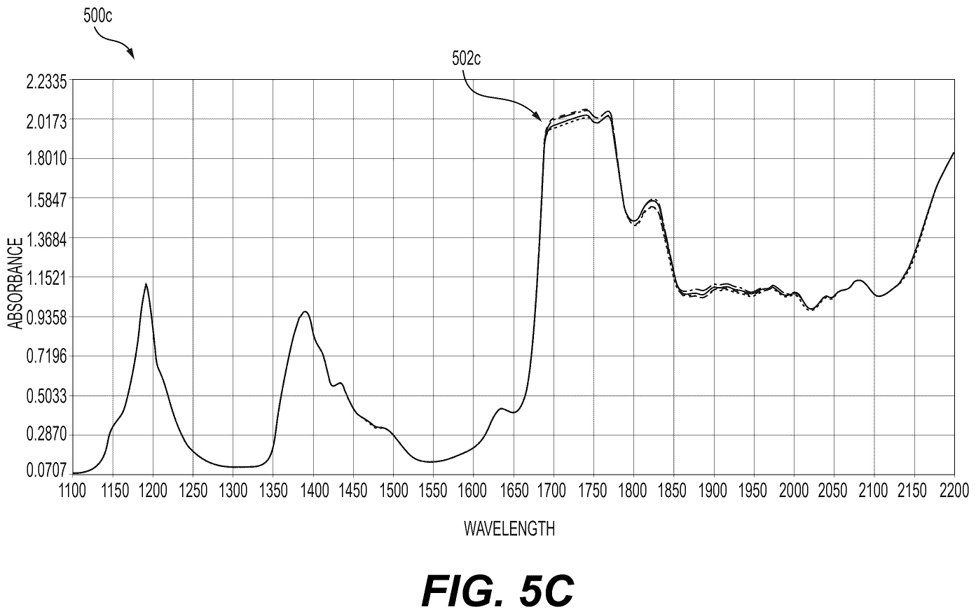

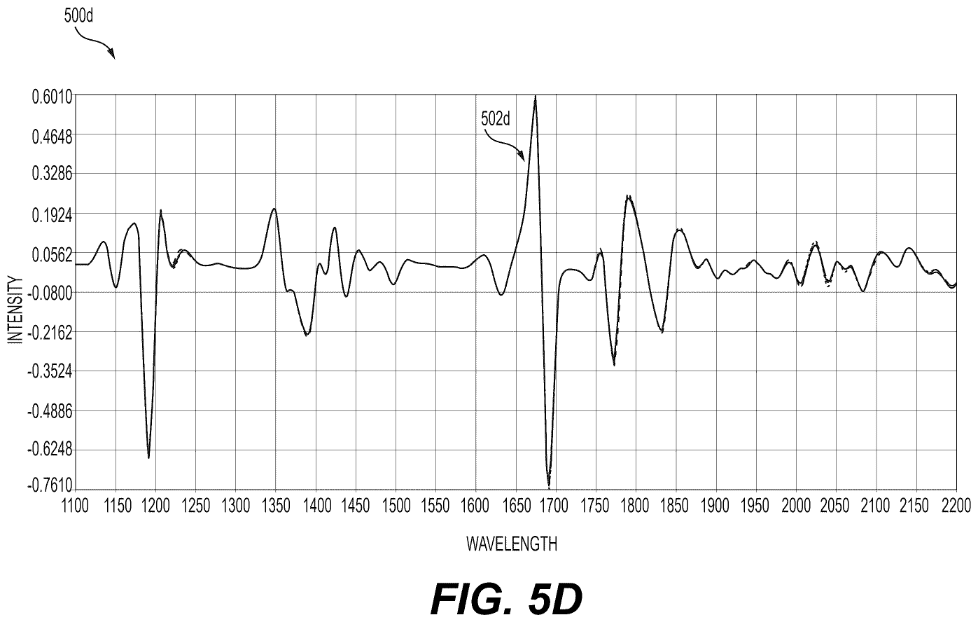

FIG. 5A is a graph 500a depicting example sample spectra 502a of multiple raw crude feedstocks obtained from one or more example spectroscopic analyzers, according to embodiments of the disclosure. In FIG. 5A, one or more wavenumber bands may be identified for wavenumbers between 4000-7500 cm⁻¹. The distinctive absorption peaks in the spectrum may, for example, represent areas where bond vibrations for specific functional groups (e.g., stretching vibrations and in-plane and out-of-plane bending modes) of a molecule occur, giving an indication of the chemical structure of the sample. The intensity and width of individual peaks may be used, at least in part, to distinguish peaks from different functional groups which occur at approximately the same position. Different wavenumber regions of the spectra may also be used to identify qualitative characteristics of the sample. The spectral response for wavenumbers greater than 7000 cm⁻¹, for example, may generally indicate asphaltenes and other heavy products, where the beam of the spectroscopic analyzer will experience greater scatter.

FIG. 5B is a graph 500b depicting a portion of the graph 500a of FIG. 5A, according to embodiments of the disclosure. FIG. 5B shows the example overlaid NIR spectra of FIG. 5A for a selected range of wavenumbers between approximately 4000 and 4750 cm⁻¹. This range of wavenumbers may be a region consistent with spectra for the stretching and bending of bonds in the characteristic functional groups of prime blending components produced for the gasoline blending pool by alkylation and other refinery operations, such as alkylate.

FIG. 5C is a graph 500c showing an example sample spectra 502c of an alkylate product obtained from one or more example spectroscopic analyzers, according to embodiments of the disclosure. The sample spectra 502c may be mathematically manipulated (e.g., a first order or higher order derivative of the sample spectra 502c may be determined, and/or statistical techniques may be applied to the sample spectra 502c) to obtain further information about the content of the alkylate product. For example, FIG. 5D is a graph 500d of the second derivative of the sample spectra 502c. The derivative spectra 502d may be obtained via various techniques (e.g., optical, electronic, mathematical, etc.).

In some embodiments, advanced process control-related (APC-related) techniques may be used to improve and/or optimize the alkylation process against processing constraints, such as, for example, processing unit capabilities. By leveraging APC-related techniques, control during the alkylation process may use control variables of the alkylation process to balance intermediates and/or products yield(s), recover the catalyst and paraffin recycle streams, determine available capacity and ratios of input streams, and/or determine available blending capacity for gasoline blending operations. Control options may be selected from multiple process variables and equipment capabilities, which may include material properties associated with the feedstocks and/or parameters associated with the feedstocks, unit materials, intermediate materials, and/or products, as well as operational parameters associated with the processing units involved in the alkylation process.

In some embodiments, advanced process control (APC)-related and/or machine-learning techniques for an alkylation process may include receiving, during a first time period of the alkylation process, a first portion of a sample stream from a point in the continuous alkylation operation (e.g., one or more intermediate or final products produced) having a plurality of components and properties. The first portion of the sample stream may be analyzed by one or more spectroscopic analyzers and generate one or more spectra from the first portion of the sample stream at one or more wavelength bands to determine the composition of the sample. Iteratively, at successive time periods of the alkylation process, the APC-related and/or machine-learning techniques may include using the one or more spectroscopic analyzers to predict the properties of a portion of the sample stream from the sampling point in the alkylation process. In some embodiments, one or more process controllers may be configured to compare and control properties, amounts, and/or process parameters according to a set of predetermined targets for the properties, amounts, and/or process parameters associated with the alkylation process. The iterative process of control may continue at successive time periods during the alkylation process, for example, until (a) deviations between (i) the predicted properties, amounts, and/or process parameters and (ii) the predetermined targets, approach zero, and/or (b) the predicted properties, amounts, and/or process parameters do not fall outside respective predetermined desired target ranges (e.g., target ranges for the one or more intermediate or final products produced). In some embodiments, the predicted properties, amounts, and/or process parameters may be controlled via the one or more process controllers to remain within respective predetermined ranges of product specifications (e.g., within maximums and/or minimums). The target ranges and product specification maximums and minimums may include, for example, predetermined specific refinery generating targets, analytical alkylation model predictions, marketing specifications, industry standard specifications, and/or other target characteristics.

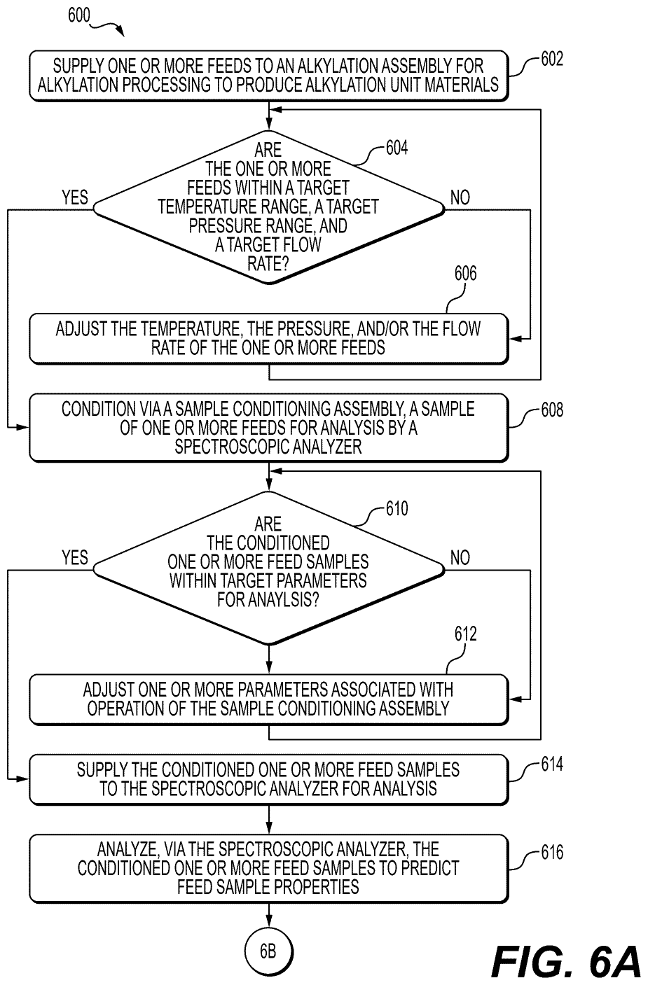

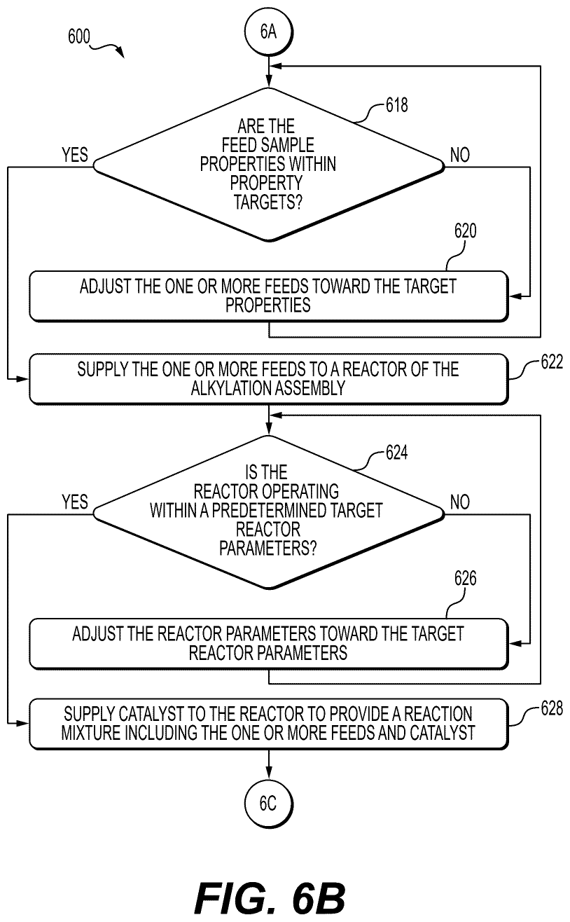

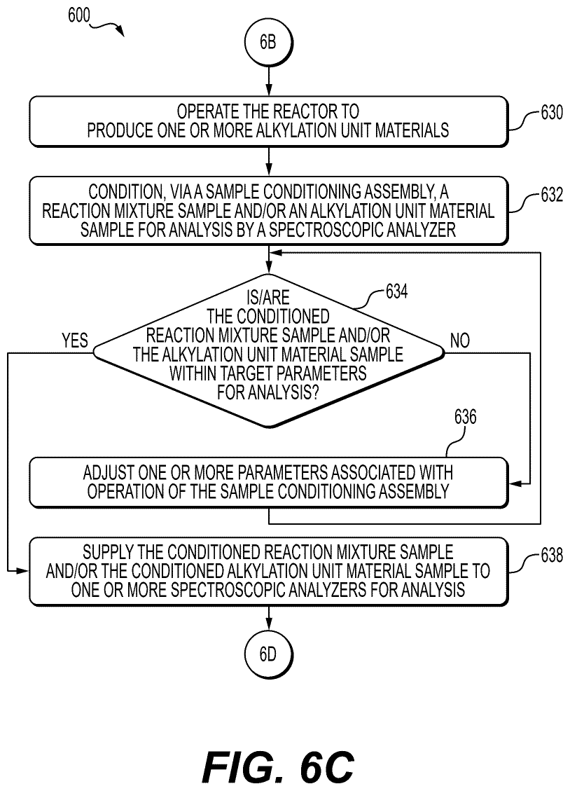

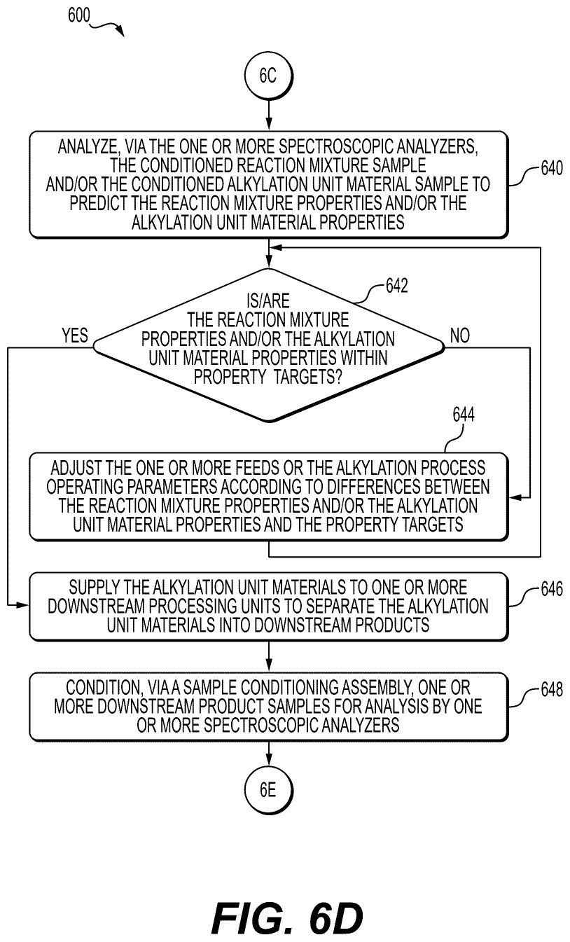

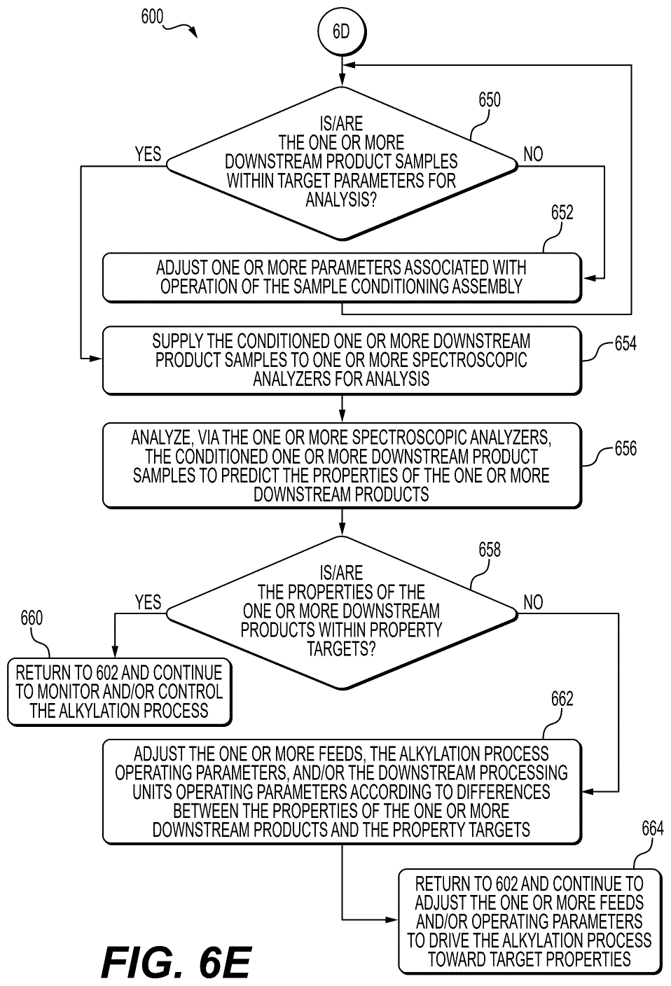

FIG. 6A, FIG. 6B, FIG. 6C, FIG. 6D, and FIG. 6E are a block diagram of an example method 600 to enhance an alkylation process associated with a refining operation, according to embodiments of the disclosure. The example method 600 is illustrated as a collection of blocks in a logical flow graph, which represent a sequence of operations. In the context of software, where applicable, the blocks represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described blocks may be combined in any order and/or in parallel to implement the method.

FIG. 6A through FIG. 6E are a block diagram of the example method 600 to enhance an alkylation process associated with a refining operation, for example, during the alkylation process, according to embodiments of the disclosure. At 602 (FIG. 6A), the example method 600 may include supplying one or more feeds to an alkylation assembly for alkylation processing to produce alkylation unit materials, for example, as described herein.

At 604, the example method 600 may include determining whether one or more feeds is within a target temperature range, a target pressure range, and/or a target flow rate, for example, as described herein.

If, at 604, it is determined that the temperature, pressure, or flow rate is not within one or more of the target ranges, at 606, the example method 600 may include adjusting the temperature, the pressure, and/or the flow rate of the one or more feeds to be within the target ranges, and returning to 604 to repeat the determination. Alternatively, or in addition, one or more correction factors (e.g., a correction factor based on past operational data) may be applied to the temperature, pressure, and/or flow rate, for example, where adjustment is difficult or not possible within the alkylation process.

If, at 604, it is determined that the temperature, pressure, or target flow rate are within the target ranges, at 608, the example method 600 may include conditioning, via a sample conditioning assembly, a sample of the one or more feeds for analysis by a spectroscopic analyzer, for example, as described herein.

At 610, the example method 600 may include determining whether the conditioned one or more feed samples are within target parameters for analysis. This may include determining whether water, particulates, and/or other contaminates have been removed from the conditioned one or more feed samples, and/or whether the conditioned samples are within a desired predetermined temperature range for improving the accuracy of the analysis by the spectroscopic analyzer(s).

If, at 610, it is determined that the conditioned one or more feed samples are not within target parameters for analysis, the example method 600, at 612, may include adjusting one or more parameters associated with operation of the sample conditioning assembly, such that the conditioned one or more feed samples are within the target parameters, and returning to 610 to repeat the determination.

If, at 610, it is determined that the conditioned one or more feed samples are within target parameters for analysis, the example method 600, at 614, may include supplying the conditioned one or more feed samples to the spectroscopic analyzer(s) for analysis, for example, as described herein.

The example method 600, at 616, may include analyzing, via the spectroscopic analyzer(s), the conditioned one or more feed samples to predict (or determine) feed sample properties (and/or parameters), for example, as described herein.

At 618 (FIG. 6B), the example method 600 may include determining whether the feed sample properties are within desired ranges of property targets for the one or more feeds.

If, at 618, it is determined that the feed sample properties are not within the desired ranges of the property targets for the one or more feeds, the example method 600, at 620, may include adjusting the one or more feeds toward the target properties to be within the desired ranges of property targets for the one or more feeds, and returning to 618 to repeat the determination.

If, at 618, it is determined that the feed sample properties are within the desired ranges of the property targets for the one or more feeds, the example method 600, at 622, may include supplying the one or more feeds to a reactor of the alkylation assembly, for example, as described herein.

At 624, the example method 600 may include determining whether the reactor is operating within a desired range of predetermined target reactor parameters.

If, at 624, it is determined that the reactor is not operating within the desired range of the predetermined target reactor parameters, the example method 600, at 626, may include adjusting the reactor parameters toward the target reactor parameters and returning to 624 to repeat the determination.

If, at 624, it is determined that the reactor is operating within the desired range of the predetermined target reactor parameters, the example method 600, at 628, may include supplying catalyst to the reactor to provide a reaction mixture including the one or more feeds and catalyst, for example, as described herein.

At 630 (FIG. 6C), the example method 600 may include operating the reactor to produce one or more alkylation unit materials, for example, as described herein.

At 632, the example method 600 may include conditioning, via a sample conditioning assembly, a reaction mixture sample and/or an alkylation unit material sample for analysis by one or more spectroscopic analyzers. In some embodiments, the one or more spectroscopic analyzers may be calibrated to generate standardized spectral responses, for example, as described herein.

At 634, the example method 600 may include determining whether the conditioned reaction mixture sample and/or the alkylation unit material sample is/are within desired ranges of target parameters for analysis. This may include determining whether water, particulates, and other contaminates have been removed from the conditioned reaction mixture sample and/or the alkylation unit material sample, and/or whether the conditioned sample is within a predetermined temperature range for improving the accuracy of the analysis by the spectroscopic analyzer.

If, at 634, it is determined that the conditioned reaction mixture sample and/or the alkylation unit material sample is/are not within the desired ranges of the target parameters for analysis, the example method 600, at 636, may include adjusting one or more parameters associated with operation of the sample conditioning assembly, such that the conditioned reaction mixture sample and/or the alkylation unit material sample is/are within the target parameters and returning to 634 to repeat the determination.

If, at 634, it is determined that the conditioned reaction mixture sample and/or the alkylation unit material sample is/are within the desired ranges of the target parameters for analysis, the example method 600, at 638, may include supplying the conditioned reaction mixture sample and/or the alkylation unit material sample to the one or more spectroscopic analyzers for analysis, for example, as described herein.

At 640 (FIG. 6D), the example method 600 may include analyzing, via the one or more spectroscopic analyzers, the conditioned reaction mixture sample and/or the conditioned alkylation unit material sample to predict (or determine) the reaction mixture properties (and/or parameters) and/or the alkylation unit material properties (and/or parameters), for example, as described herein. In some embodiments, the one or more spectroscopic analyzers may be calibrated to generate standardized spectral responses, for example, as described herein.

At 642, the example method 600 may include determining whether the reaction mixture properties and/or the alkylation unit material properties is/are within desired ranges of respective property targets.

If, at 642, it is determined that the reaction mixture properties and/or the alkylation unit material properties is/are not within the desired ranges of the respective property targets, the example method 600, at 644, may include altering one or more of the one or more feeds or the alkylation process operating parameters according to differences between the reaction mixture properties and/or the alkylation unit material properties and the property targets, and returning to 642 to repeat the determination.

If, at 642, it is determined that the reaction mixture properties and/or alkylation unit material properties is/are within the desired ranges of the respective property targets, the example method 600, at 646, may include supplying the alkylation unit materials to one or more downstream processing units to separate the alkylation unit materials into downstream products, for example, as described herein.

At 648, the example method 600 may include conditioning, via a sample conditioning assembly, one or more downstream product samples for analysis by one or more spectroscopic analyzers, for example, as described herein.

At 650 (FIG. 6E), the example method 600 may include determining whether the conditioned one or more downstream product samples is/are within desired ranges of target parameters for analysis. This may include determining whether water, particulates, and other contaminates have been removed from the conditioned one or more downstream product samples, and/or whether the conditioned samples is/are within a desired predetermined temperature range for improving the accuracy of the analysis by the spectroscopic analyzer.

If, at 650, it is determined that the conditioned one or more downstream product samples is/are not within the desired ranges of the target parameters for analysis, the example method 600, at 652, may include adjusting one or more parameters associated with operation of the sample conditioning assembly such that the conditioned one or more downstream product samples is/are within the desired ranges of the target parameters, and returning to 650 to repeat the determination.

If, at 650, it is determined that the conditioned one or more downstream product samples is/are within the desired ranges of the target parameters for analysis, the example method 600, at 654, may include supplying the conditioned one or more downstream product samples to the one or more spectroscopic analyzers for analysis, for example, as described herein.

At 656, the example method 600 may include analyzing, via the one or more spectroscopic analyzers, the conditioned one or more downstream product samples to predict the properties (and/or parameters) of the one or more downstream products, for example, as described herein. In some embodiments, the one or more spectroscopic analyzers may be calibrated to generate standardized spectral responses, for example, as described herein.

At 658, the example method 600 may include determining whether the properties of the one or more downstream products are within desired ranges of property targets.

If, at 658, it is determined that the properties of the one or more downstream products are not within the desired ranges of the property targets, the example method 600, at 660, may include adjusting one or more of the one or more feeds, the alkylation process operating parameters, or the downstream processing units operating parameters according to differences between the properties of the one or more downstream products and the property targets, for example, as described herein. Thereafter, at 662, the example method 600 may include returning to 602 and continuing to adjust the hydrocarbon feedstock and/or operating parameters to drive the alkylation process toward target properties.

If, at 658, it is determined that the properties of the one or more downstream products are within the desired ranges of the property targets, the example method 600, at 664, may include returning to 602 and continuing to monitor and/or control the alkylation process according to the example method 600.

In some embodiments, the example method 600 may result in causing the alkylation process to produce one or more of: intermediate materials having one or more properties within a selected range of one or more target properties of the intermediate materials, unit materials having one or more properties within a selected range of one or more target properties of the unit materials, or downstream materials having one or more properties within a selected range of one or more target properties of the downstream materials. In some embodiments, this may cause the alkylation process to achieve material outputs that more accurately and responsively converge on one or more of the target properties. In some embodiments, the example method may result in optimizing one or more of: (a) one or more target properties of the one or more intermediate materials, (b) one or more target properties of the one or more unit materials, (c) or one or more target properties of one or more downstream materials produced by the one or more second processing units or downstream processing units, thereby to optimize the alkylation process to achieve material outputs that more accurately and responsively converge on one or more of the target properties.

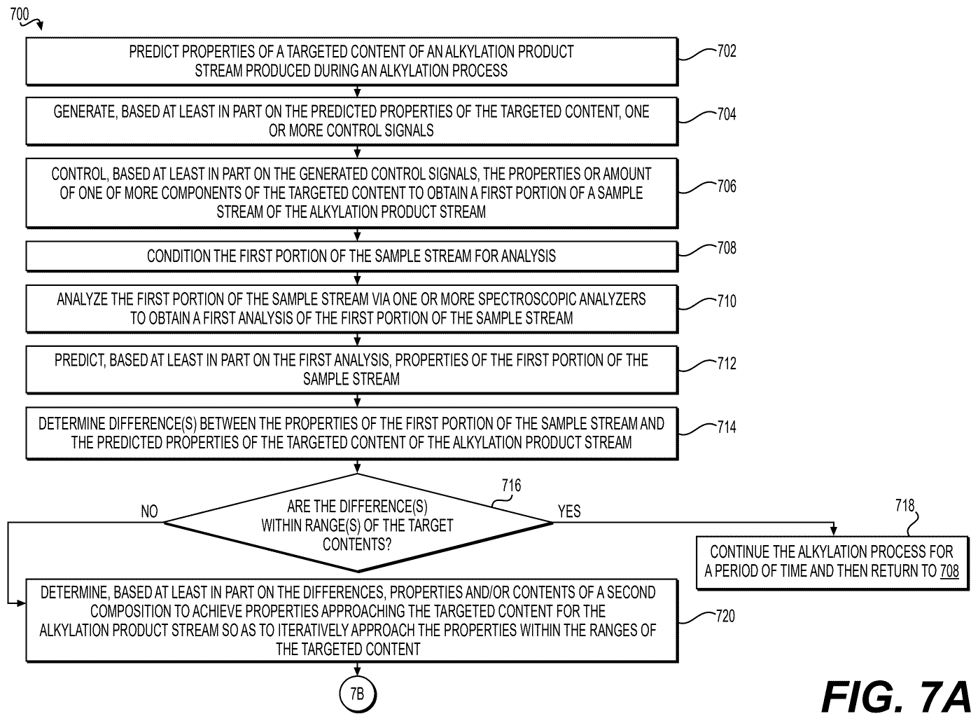

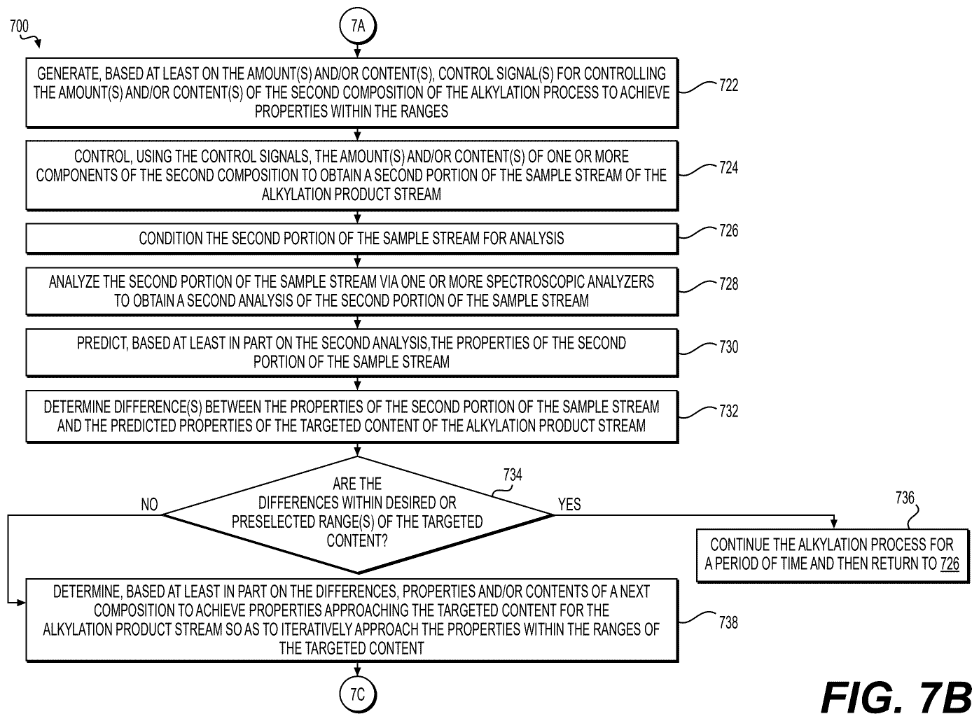

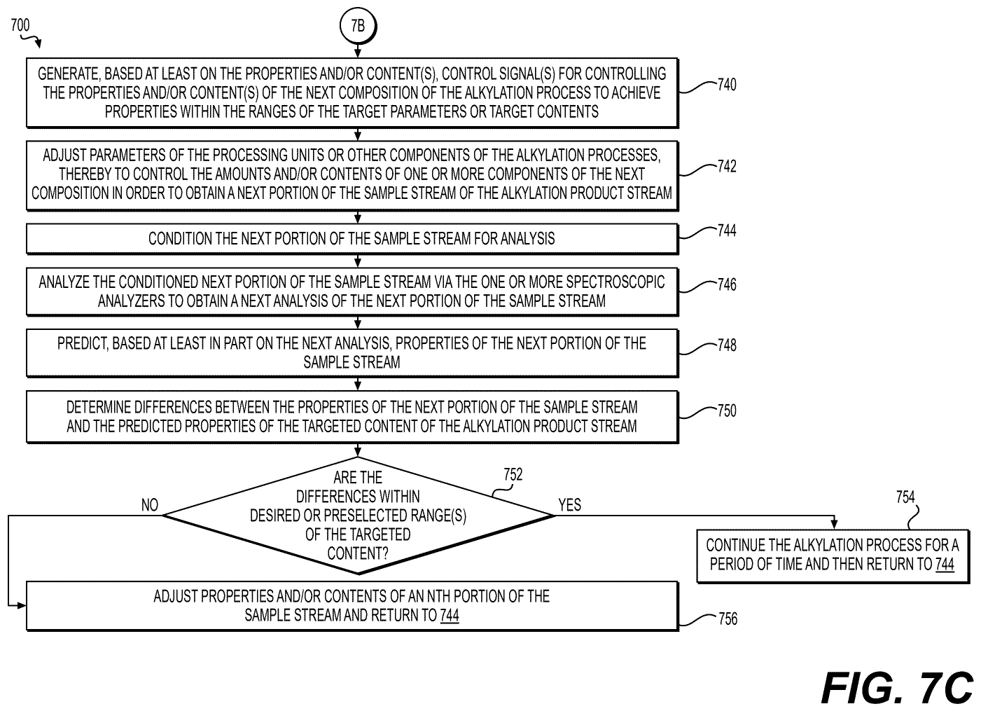

FIG. 7A, FIG. 7B, and FIG. 7C are a block diagram illustrating an example method 700 for enhancing an alkylation process, according to embodiments of the disclosure. The example method 700 is illustrated as a collection of blocks in a logical flow graph, which represent a sequence of operations. In the context of software, where applicable, the blocks represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described blocks may be combined in any order and/or in parallel to implement the method.

As shown in FIG. 7A, example method 700, at 702, may include predicting the properties of targeted properties and/or a targeted content of an alkylation product stream produced during an alkylation process. The alkylation product stream may include, for example, an alkylate product coming from a debutanizer unit. In some examples, the targeted content of the alkylate may be predicted by an analytical model of the alkylation process based on desired recipes for, for example, a gasoline blending operation downstream of the alkylation process. For example, the targeted content of the alkylate may be selected to achieve one or more targeted blended gasoline fuel properties of a blended gasoline fuel recipe, with the alkylate product blended to achieve a preselected target gasoline blend. In some embodiments, the analytical model may include machine-learning capabilities (e.g., the ability to be self-trained, for example, on past, current, and/or future operational data), for example, such that the analytical model is periodically, intermittently, and/or continuously updated and/or improved.

In some examples, the alkylation product stream may include a propane product stream, a paraffin recycle stream, a catalyst recycle stream, and/or other product stream having a targeted content, which may result in improvement of the efficiency of at least a portion of the alkylation process.

At 704, the example method 700 may include generating one or more control signals based at least in part on the predicted properties of the targeted content. For example, the control signals may be generated by one or more process controllers and may be communicated to one or more processing units and/or other refinery components (e.g., pumps, valves, heat exchangers, and/or other equipment), for example, as described herein.

In some embodiments, at 706, example method 700 may include using the generated control signals in controlling the properties or amount of one or more components of the targeted content (e.g., by adjusting feedstocks and/or parameters of the alkylation process) to obtain a first portion of a sample stream from the alkylation product stream. Control variables may include, for example, one or more of (a) content, (b) properties, or (c) parameters, of the unit materials, unit product materials, intermediate materials, and/or another stream from within the alkylation process.

A first portion of a sample stream from the alkylation process may be obtained for analysis during a first time period. At 708, the example method 700 may include conditioning the first portion of the sample stream, for example, as described herein. The conditioning may include using a sample conditioning assembly, such as, for example, the example sample conditioning assembly 202 shown in FIG. 4, to filter, degas, and/or change the temperature of the first portion in preparation for analysis by the one or more spectroscopic analyzers.

The example method 700, at 710, may further include analyzing the first portion of the sample stream via the one or more spectroscopic analyzers, and, at 712, the example method 700 may include predicting first properties for the first portion of the sample stream. The analysis may include measuring absorbance, transmission, reflectance, attenuated total reflectance, and/or scattering intensity associated with the conditioned first portion of the sample stream, and generating one or more spectra for the first portion at one or more wavelength bands.

The example method 700, at 714, may further include determining differences between the properties of the first portion of the sample stream and the predicted properties of the targeted content of the alkylation product steam. In some embodiments, the targeted content may include a predetermined target composition or a dynamically modeled target composition, for example, as described herein. At 716, the example method 700 may include determining whether the differences are within range(s) of preselected target properties and/or within range(s) of the target contents.

If, at 716 the determined differences are within range(s) of the targeted contents, at 718, the example method 700 may continue the alkylation process for a period of time and thereafter return to 708. If at, 716, the determined differences are not within range(s) of the targeted contents, at 720, the example method 700 may include determining, based at least in part on the differences, properties and/or contents of a second composition to achieve properties at least approaching the targeted composition for the alkylation product stream, for example, so as to iteratively approach properties within the ranges of the preselected targeted content.

Referring to FIG. 7B, at 722, the example method 700 may include generating control signals for controlling the amounts and/or contents of the second composition of the alkylation process to achieve properties within the range(s) of the targeted content (e.g., the control signals may be generated to change parameters of the processing units or other components of the alkylation operation). In some embodiments, generating the control signals may include first converting measurement signals from the sample spectra into output signals indicative of the properties of the sample stream.

The example method 700, at 724, may thereafter include controlling (e.g., during a second time period after the first time period) the amounts and/or contents of the second composition by changing parameters of the processing units of the alkylation operation in order to obtain a second portion of the sample stream of the alkylation product stream (e.g., during a second iteration of the method). The second portion of the sample stream may have different composition, properties, and/or contents than the first portion and may be analyzed during the second time after the first time portion.

At 726, the example method 700 may include conditioning the second portion of the sample stream, for example, as described herein. The conditioning may include using a sample conditioner assembly to filter, degas, and/or change the temperature of the second portion in preparation for analysis by the one or more spectroscopic analyzers.

At 728, the example method 700 may include analyzing the second portion of the sample stream via the one or more spectroscopic analyzers to obtain a second analysis and set of spectra, which may be used, at 730, to predict the properties and/or contents of the second portion of the sample stream.

The example method 700, at 732, further may include determining differences between the targeted contents of the alkylation product stream and the predicted properties and/or contents of the second portion of the sample stream. At 734, the example method 700 may include determining whether the differences are within preselected range(s) the targeted content.

If, at 734, the determined differences are within the preselected range(s) of preselected targeted content, at 736, the example method 700 may continue the alkylation process for a period of time and thereafter return to 726. If, at 734, the determined differences are not within preselected range(s) of the targeted content, at 738, the example method 700 may include determining, based at least in part on the differences, properties and/or amounts of a next composition (e.g., for a next iteration of the method) of the alkylate product to achieve properties at least approaching properties within the preselected ranges of the targeted content, so as to iteratively approach properties within the ranges of the targeted content.

Referring to FIG. 7C, at 740, the example method 700 may include generating control signals for controlling the amounts and/or contents of the next composition of the alkylation process to achieve properties within the ranges (e.g., during a second time period), which may include, for example, adjusting parameters and/or operational characteristics of the processing units of the alkylation processes, for example, as described herein. In some embodiments, generating the control signals may include converting measurement signals from the sample spectra into output signals indicative of the properties of the sample stream determined from the second composition and/or the next composition.

The example method 700, at 742, may thereafter include changing parameters of the processing units or other components of the alkylation processes, thereby to control the amounts, properties, and/or contents of one or more components of the next composition in order to obtain a next portion of the sample stream of the alkylation product stream (e.g., during a further iteration of the method). In some embodiments, later-occurring iterations of the alkylate product may have different properties and/or different compositions as compared to the first composition of the alkylate product and/or the second composition of the alkylate product and may be analyzed at a next time period after the first time period and the second time period.

At 744, the example method 700 may include conditioning the next portion of the sample stream, for example, as described herein. The conditioning may include using a sample conditioner assembly to filter, degas, and/or change the temperature of the second portion in preparation for analysis by the one or more spectroscopic analyzers.

At 746, the example method 700 may further include analyzing the conditioned sample stream via the one or more spectroscopic analyzers to generate a set of spectra, which may be used, at 748, to predict properties of the next portion of the sample stream.

At 750, the example, method 700 may include determining differences between the predicted properties of the targeted content of the alkylation product stream and the predicted properties of the next portion of the sample stream. At 752, the example method 700 further may include determining whether differences between the predicted properties of the targeted content of the alkylation product stream and the predicted properties of the next portion of the sample stream are within the desired or preselected range(s) of the targeted content, for example, during the next time period, following the second time period.

If, at 752, the determined differences are within preselected range(s) of the targeted content, at 754, the example method 700 may include continuing the alkylation processes for a period of time, and thereafter returning to 744. If, at 752, the determined differences are not within preselected range(s) of the targeted content, at 756, the example method 700 may include continuing to adjust properties and/or contents of the next composition (e.g., until an nth iteration), based at least in part on the differences, to achieve properties nearer to the targeted content for the alkylation product stream. In this example manner, the method 700 may iteratively achieve properties within the preselected ranges of the preselected target parameters and/or target content, and/or within preselected ranges of new target properties and/or new target contents, for example, determined during a subsequent time period. New target properties and/or contents may be introduced (e.g., on-line) during the alkylation process and/or may result from, at least in part, for example, revised gasoline blending recipes, changes in plant equipment or setup, changes to the layout of the alkylation process, and/or to changes in other process and/or processing unit parameters.

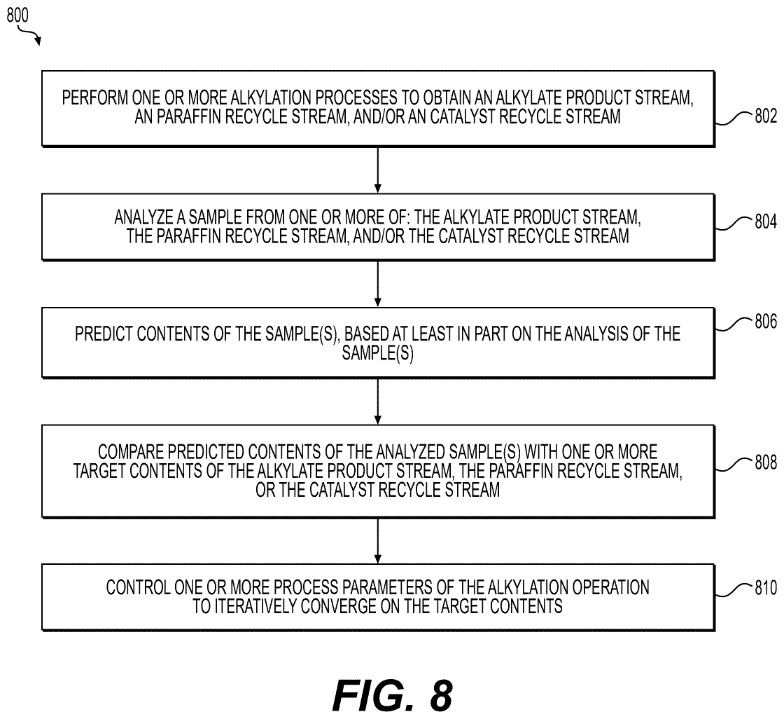

FIG. 8 is a block diagram illustrating another example process 800 for enhancing an alkylation process using example iterative APC-related techniques (e.g., for producing one or more alkylates for use in gasoline blending operations), according to embodiments of the disclosure. The alkylate(s) may have a plurality of targeted properties. The example method 800 may include performing, at 802, one or more alkylation processes to obtain an alkylate product stream, a paraffin recycle stream, and a catalyst recycle stream, for example, as described herein. The plurality of targeted properties may be based at least in part on, for example, results from operation of a predictive analytical model (e.g., such as the analytical alkylation model 212, as shown in FIG. 3). The results may be obtained, based at least in part, on actual data and/or training data (e.g., laboratory-derived data) that may include a set of respective spectra and associated properties from samples. The analytical model may be, for example, an alkylation model that may be a machine-learning-trained model trained and updated using, for example, alkylation process data.

The example method 800, at 804, may include analyzing one or more samples obtained from one or more output product streams from the alkylation process. For example, one or more samples may be analyzed from one or more of the alkylate product stream, the paraffin recycle stream, or the catalyst recycle stream. At 806, the example method 800 may include predicting the properties of the samples (e.g., one or more products included in the product streams). In some embodiments, the properties may be correlated to indications of the quality and/or efficiency of the alkylation processes. In some embodiments, the analyzing may be performed via one or more spectroscopic analyzers, for example, as described herein. In some embodiments, the analyzing may include conditioning sample streams to provide conditioned samples to the one or more spectroscopic analyzers, such as filtering, degassing, and/or controlling temperature associated with the sample streams (e.g., the sample streams and/or the surroundings), for example, via a sample conditioning assembly 202, as shown in FIG. 4. For example, a first sample stream may be conditioned during the first time cycle, a second sample stream may be conditioned during a second time cycle, and an nth sample stream may be conditioned during an nth time cycle, to filter, degas, dilute, and/or control the temperature of the sample stream to be within a preselected temperature range.

Prior to, during, and/or after the alkylation process, property and/or contents of the alkylate product from the alkylate product stream may be determined, for example, for the mixing of one or more blended fuel product recipes. The blended fuel product recipes may be produced, for example, by adding various amounts of the alkylate product from the alkylate product stream to one or more base fuel mixtures. In some embodiments, the determined properties and/or contents may be achieved by controlling the alkylation through, for example, (a) the concentration of a catalyst recycle product in the catalyst recycle stream; (b) the amount of water in the catalyst recycle product of the catalyst recycle stream; (c) the amount of acid oil in the catalyst recycle product of the catalyst recycle stream; (d) the concentrations in a paraffin recycle product of the paraffin recycle stream; and/or (e) the target composition of the alkylation product or products.

At 808, the example method 800 may include comparing the contents of the analyzed one or more samples with one or more target contents of the alkylation product stream, such as target contents associated with the alkylate product stream, the paraffin recycle stream, and/or the catalyst recycle stream. The target contents may be derived from, for example, the analytical model, from predicted blended gasoline fuel properties, and/or another source. The example method 800 may continue at 810 and include controlling one or more process parameters of the alkylation operation in order to iteratively converge or the target contents. For example, ongoing sample analysis and control may allow the alkylation process to converge on the target contents. The one or more process parameters may include, for example, operational setpoints and/or operational conditions and constraints associated with one or more processing units of the alkylation operation, or upstream of the alkylation operation, or downstream of the alkylation operation, to cause the predicted contents to converge on the target contents.

The example method 800 may additionally include generating one or more control signals representative of changes to the process parameters. The generated control signals may be converted to output signals by receiving the generated signals indicative of the measured absorbance, transmission, reflectance, attenuated total reflectance, or scattering intensity associated with the sample stream and generating and processing a set of spectra based on the received signals.

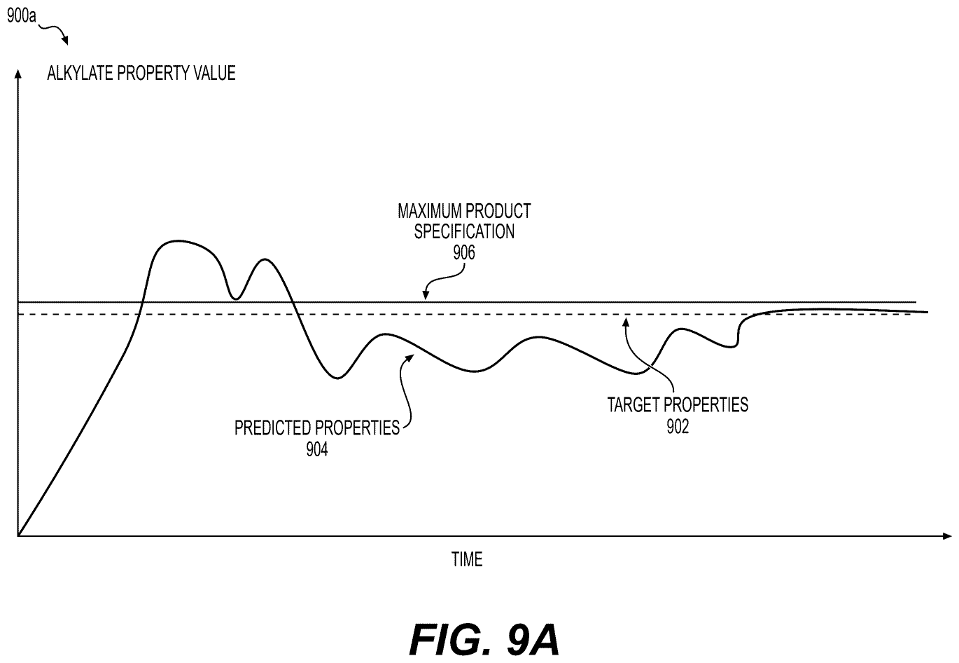

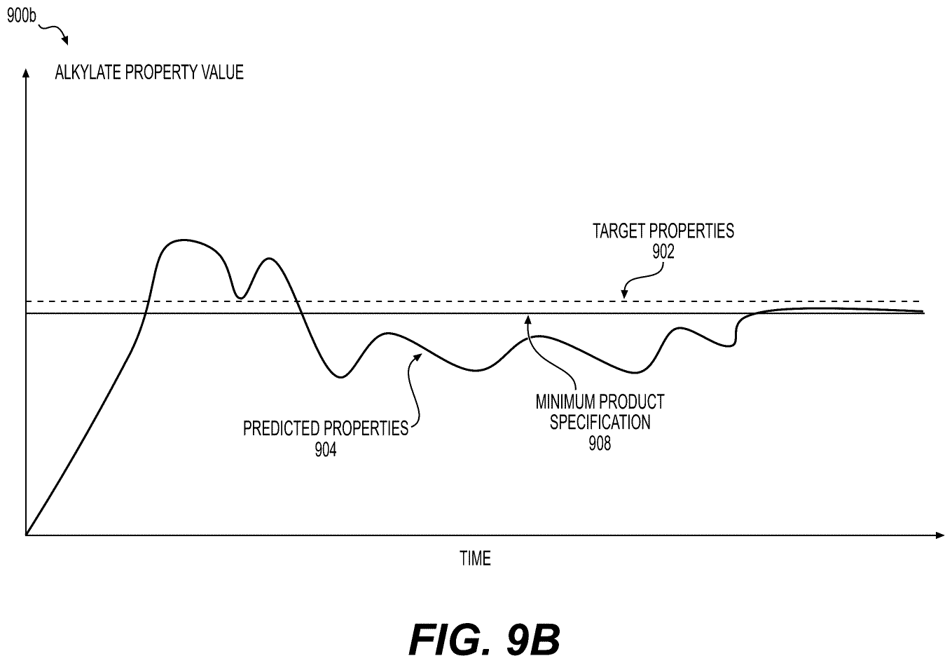

FIG. 9A is an example graph 900a showing one or more example alkylate predicted properties 904 as a function of time during an example alkylation process, according to embodiments of the disclosure. The iterative alkylation processes, according to some embodiments, as described herein, may continue until the differences between the predicted properties 904 of an alkylate product and the preselected (or modeled) target properties 902 are approach to zero (e.g., within a preselected range of zero), and the predicted properties 904 do not exceed a preselected maximum product specification 906 of the alkylate product (see FIG. 9A).

FIG. 9B is another example graph 900b showing an example one or more alkylate predicted properties 904 as a function of time during an example alkylation process, according to embodiments of the disclosure. For example, as depicted in FIG. 9B, the predicted properties 904 of the alkylate product may be controlled to converge on one or more target properties 902, for example, within a predetermined range of the product specification, while not exceeding a maximum product specification 906 (FIG. 9A) or a minimum product specification 908 (FIG. 9B) of the alkylate product. The product specification parameters and/or maximum and/or minimum values may be developed based at least in part from an analytical model, current refining production targets, and/or other factors. In some embodiments, the analytical model may include a machine-learning-trained model, and the one or more target properties 902 may be iteratively updated during successive time cycles associated with the process, for example, with process data from an alkylation operation, as described herein.

In some embodiments, sample properties predicted by the spectroscopic analyzers, operating parameters of the processing units, the maximum and minimum product specifications and/or other information may be output to one or more output devices, for example, as discussed herein. The output device may include, for example, a display in communication with at least one of the one or more spectroscopic analyzers, controllers, and/or processing units engaged in the one or more alkylation processes. In some embodiments, the one or more output devices may update sample properties and/or operating parameters, for example, in real-time, and/or may include visual and/or audio alarms associated with target or threshold values for the sample properties or operating parameters.

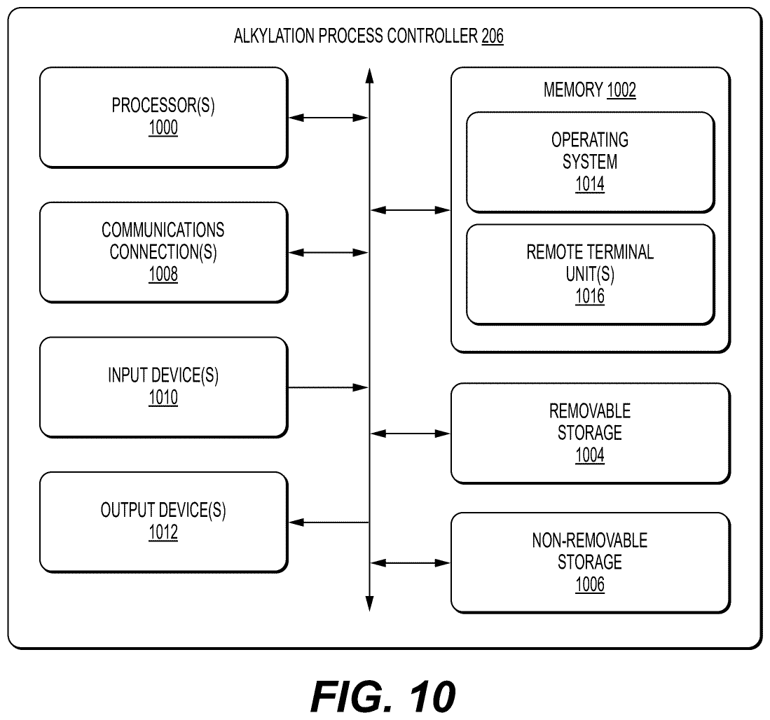

FIG. 10 is a schematic diagram of an example alkylation process controller 206 configured to at least partially control an alkylation process 12 according to embodiments of the disclosure, for example, as described herein. The alkylation process controller 206 may include one or more processor(s) 1000 configured to execute certain operational aspects associated with implementing certain systems and methods described herein. The processor(s) 1000 may communicate with a memory 1002. The processor(s) 1000 may be implemented and operated using appropriate hardware, software, firmware, or combinations thereof. Software or firmware implementations may include computer-executable or machine-executable instructions written in any suitable programming language to perform the various functions described. In some examples, instructions associated with a function block language may be stored in the memory 1002 and executed by the processor(s) 1000.

The memory 1002 may be used to store program instructions that are loadable and executable by the processor(s) 1000, as well as to store data generated during the execution of these programs. Depending on the configuration and type of the alkylation process controller 206, the memory 1002 may be volatile (such as random access memory (RAM)) and/or non-volatile (such as read-only memory (ROM), flash memory, etc.). In some examples, the memory devices may include additional removable storage 1004 and/or non-removable storage 1006 including, but not limited to, magnetic storage, optical disks, and/or tape storage. The disk drives and their associated computer-readable media may provide non-volatile storage of computer-readable instructions, data structures, program modules, and other data for the devices. In some implementations, the memory 1002 may include multiple different types of memory, such as static random access memory (SRAM), dynamic random access memory (DRAM), or ROM.

The memory 1002, the removable storage 1004, and the non-removable storage 1006 are all examples of computer-readable storage media. For example, computer-readable storage media may include volatile and non-volatile, removable and non-removable media implemented in any method or technology for storage of information such as computer-readable instructions, data structures, program modules or other data. Additional types of computer storage media that may be present may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile discs (DVD) or other optical storage, magnetic cassettes, magnetic tapes, magnetic disk storage or other magnetic storage devices, or any other medium which may be used to store the desired information and which may be accessed by the devices. Combinations of any of the above should also be included within the scope of computer-readable media.

The alkylation process controller 206 may also include one or more communication connection(s) 1008 that may facilitate a control device (not shown) to communicate with devices or equipment capable of communicating with the alkylation process controller 206. The alkylation process controller 206 may also include a computer system (not shown). Connections may also be established via various data communication channels or ports, such as USB or COM ports to receive cables connecting the alkylation process controller 206 to various other devices on a network. In some examples, the alkylation process controller 206 may include Ethernet drivers that enable the alkylation process controller 206 to communicate with other devices on the network. According to various examples, communication connections 1008 may be established via a wired and/or wireless connection on the network.

The alkylation process controller 206 may also include one or more input devices 1010, such as a keyboard, mouse, pen, voice input device, gesture input device, and/or touch input device. It may further include one or more output devices 1012, such as a display, printer, and/or speakers. In some examples, computer-readable communication media may include computer-readable instructions, program modules, or other data transmitted within a data signal, such as a carrier wave or other transmission. As used herein, however, computer-readable storage media may not include computer-readable communication media.

Turning to the contents of the memory 1002, the memory 1002 may include, but is not limited to, an operating system (OS) 1014 and one or more application programs or services for implementing the features and embodiments disclosed herein. Such applications or services may include remote terminal units 1016 for executing certain systems and methods for controlling operation of the alkylation process 12 (e.g., semi- or fully-autonomously controlling operation of the alkylation process 12), for example, upon receipt of one or more control signals generated by the alkylation process controller 206. In some embodiments, one or more remote terminal unit(s) 1016 may be located in the vicinity of the alkylation processing units. The remote terminal unit(s) 1016 may reside in the memory 1002 or may be independent of the alkylation process controller 206. In some examples, the remote terminal unit(s) 1016 may be implemented by software that may be provided in configurable control block language and may be stored in non-volatile memory. When executed by the processor(s) 1000, the remote terminal unit(s) 1016 may implement the various functionalities and features associated with the alkylation process controller 206 described herein.

As desired, embodiments of the disclosure may include an alkylation process controller 206 with more or fewer components than are illustrated in FIG. 10. Additionally, certain components of the example alkylation process controller 206 shown in FIG. 10 may be combined in various embodiments of the disclosure. The alkylation process controller 206 of FIG. 10 is provided by way of example only.

References are made to block diagrams of systems, methods, apparatuses, and computer program products according to example embodiments. It will be understood that at least some of the blocks of the block diagrams, and combinations of blocks in the block diagrams, may be implemented at least partially by computer program instructions. These computer program instructions may be loaded onto a general purpose computer, special purpose computer, special purpose hardware-based computer, or other programmable data processing apparatus to produce a machine, such that the instructions which execute on the computer or other programmable data processing apparatus create means for implementing the functionality of at least some of the blocks of the block diagrams, or combinations of blocks in the block diagrams discussed.

These computer program instructions may also be stored in a non-transitory computer-readable memory that may direct a computer or other programmable data processing apparatus to function in a particular manner, such that the instructions stored in the computer-readable memory produce an article of manufacture including instruction means that implement the function specified in the block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide task, acts, actions, or operations for implementing the functions specified in the block or blocks.

One or more components of the systems and one or more elements of the methods described herein may be implemented through an application program running on an operating system of a computer. They may also be practiced with other computer system configurations, including hand-held devices, multiprocessor systems, microprocessor-based or programmable consumer electronics, mini-computers, mainframe computers, and the like.

Application programs that are components of the systems and methods described herein may include routines, programs, components, data structures, etc. that may implement certain abstract data types and perform certain tasks or actions. In a distributed computing environment, the application program (in whole or in part) may be located in local memory or in other storage. In addition, or alternatively, the application program (in whole or in part) may be located in remote memory or in storage to allow for circumstances where tasks may be performed by remote processing devices linked through a communications network.

Examples of enhanced control of an alkylation process are provided below. Properties and contents may be determined from the analysis of the unit materials, unit product materials, intermediate materials, downstream materials, alkylation products, and/or other materials from the material streams, as described herein, and are given as examples to supplement those already provided and are not intended to be construed as an exhaustive list.

According to some embodiments, by measuring and determining the properties of the isobutane recycle stream, the isobutane feedstock purity may be controlled to optimize the isobutane/olefin ratio and charge rate to the alkylation process. Alternatively, control of the isobutane purity may be optimized to control the octane of the alkylate product.

According to some embodiments, the determination of alkylate octane produced during the alkylation process may allow the olefin charge rate to be optimized up to specified alkylate octane constraints. In some embodiments, alkylate octane constraints may be dictated, at least in part, by gasoline blending preferences and/or alkylate supply requirements, for example, to optimize a refinery-wide naphtha pool.

According to some embodiments, amylene content in the feed to at least some alkylation processes may be optimized, for example, while minimizing C6+ content in the olefin feed. In some embodiments, less C6+ content in the feed may reduce contaminants in the processing units and allow increased feed rates and/or reduced catalyst consumption, and/or may prevent catalyst settling issues in the catalyst settler unit.

According to some embodiments, control of the strength/concentration of the catalyst recycle stream (e.g., the acid catalyst recycle stream) may be used to optimize (e.g., maximize) the catalyst spending range while avoiding, for example, acid runaway. For example, controlling the strength of an acid catalyst may also allow for acid staging for different olefin feeds, thereby permitting improved management of upstream processing units.

According to some embodiments, the control of catalyst purity (e.g., acid catalyst purity) within a predetermined range may optimize (e.g., maximize) alkylate octane and/or yield, for example, up to the constraints of the acid regeneration process.

According to some embodiments, control of the water content in feeds and/or other material streams may suppress or prevent corrosion in alkylation processing units and/or other facility components, for example, while promoting or ensuring sufficient moisture content is available to reduce or prevent alkylate octane loss. Identifying the water content in different alkylation material streams may also be used as a maintenance/replacement indicator for some components.

According to some embodiments, the Reid Vapor Pressure (RVP) of different material streams may be used in APC-related applications for individual processing units. In some embodiments, RVP may be used as a control factor, for example, to optimize C4 content (e.g., maximize or minimize C4 content), depending on the product desired.

According to some embodiments, the boiling point of different material streams (e.g., feedstocks or products) may be used in APC-related applications for individual processing units. For example, the boiling point may be used in refinery-wide Naphtha pool optimization, and/or to prepare various material streams for acceptable levels of impurities allowed in downstream processing units.

According to some embodiments, a detailed feed analysis of substantially pure components in material feeds may facilitate optimization of the alkylation process, for example, via prediction of catalyst consumption and/or alkylate octane. In some embodiments, pure components in the paraffin recycle stream (e.g., an isobutane recycle stream) may be controlled, for example, to minimize propane and nC4 levels for pressure control and/or increased charge for the paraffin feeds.

Having now described some illustrative embodiments of the disclosure, it should be apparent to those skilled in the art that the foregoing is merely illustrative and not limiting, having been presented by way of example only. Numerous modifications and other embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives. Those skilled in the art should appreciate that the parameters and configurations described herein are exemplary and that actual parameters and/or configurations will depend on the specific application in which the systems and techniques of the disclosure are used. Those skilled in the art should also recognize or be able to ascertain, using no more than routine experimentation, equivalents to the specific embodiments of the disclosure. It is, therefore, to be understood that the embodiments described herein are presented by way of example only and that, within the scope of any appended claims and equivalents thereto, the disclosure may be practiced other than as specifically described.

The present disclosure describes examples of system and methods for improved alkylation controls according to any of the following examples:
Clause 1. A method for enhancing control of an alkylation process associated with a refining operation, the method comprising: analyzing, via a first spectroscopic analyzer to provide olefin feed sample spectra, an olefin feed sample of an olefin feed supplied to an alkylation unit positioned to perform at least a portion of the alkylation process, the olefin feed having one or more olefin feed properties; analyzing, via one of the first spectroscopic analyzer or a second spectroscopic analyzer to provide paraffin feed sample spectra, a paraffin feed sample of a paraffin feed supplied to the alkylation unit, the paraffin feed having one or more paraffin feed properties; predicting one or more olefin feed sample properties associated with the olefin feed based at least in part on the olefin feed sample spectra; predicting one or more paraffin feed sample properties associated with the paraffin feed based at least in part on the paraffin feed sample spectra; processing, via the alkylation unit, the olefin feed, the paraffin feed, and one or more catalysts to produce one or more corresponding unit materials, the one or more corresponding unit materials comprising one or more of intermediate materials or unit product materials; analyzing a unit material sample, via one of the first spectroscopic analyzer, the second spectroscopic analyzer, or a third spectroscopic analyzer, to provide unit material sample spectra; predicting one or more unit material sample properties associated with the unit material sample based at least in part on the unit material sample spectra; and prescriptively controlling, during the alkylation process, via one or more controllers based at least in part on the one or more olefin feed sample properties, the one or more paraffin feed sample properties, and the one or more unit material sample properties, one or more of: (a) one or more olefin feed properties associated with the olefin feed supplied to the alkylation unit; (b) one or more paraffin feed properties associated with the paraffin feed supplied to the alkylation unit; (c) one or more intermediate properties associated with the intermediate materials produced by the alkylation unit; (d) one or more unit product properties associated with the unit product materials produced by the alkylation unit; (e) operation of the alkylation unit; (f) operation of one or more first processing units positioned upstream relative to the alkylation unit; or (g) operation of one or more second processing units positioned downstream relative to the alkylation unit, so that the prescriptively controlling causes the alkylation process to produce one or more of: (i) one or more intermediate materials each having one or more properties within a range of one or more target properties of the one or more intermediate materials, (ii) one or more unit product materials each having one or more properties within a range of one or more target properties of the one or more unit product materials, or (iii) one or more downstream materials produced by the one or more second processing units, the one or more downstream materials each having one or more properties within a range of one or more target properties of the one or more downstream materials, thereby to cause the alkylation process to achieve material outputs that more accurately and responsively converge on one or more of the target properties.
Clause 2. The method of clause 1, further comprising prescriptively controlling content of the one or more downstream materials via control of one or more of: (a) content of the olefin feed supplied to the alkylation unit; (b) content of the paraffin feed supplied to the alkylation unit; (c) content of the one or more catalysts processed by the alkylation unit; (d) the operation of the alkylation unit; (e) content of the intermediate materials produced by the alkylation unit; (f) content of the unit product materials produced by the alkylation unit; (g) the operation of the one or more first processing units; or (h) the operation of the one or more second processing units.
Clause 3. The method of clause 1, wherein the prescriptively controlling comprises operating a prescriptive analytical model configured to improve an accuracy of one or more of: (a) the one or more olefin feed properties; (b) the one or more paraffin feed properties; (c) content of the one or more of the unit materials; (d) purity of the one or more catalysts; or (e) the downstream materials produced by the one or more second processing units.
Clause 4. The method of clause 1, wherein the prescriptively controlling operation of one or more of (a) the one or more first processing units, (b) the alkylation unit, or (c) the one or more second processing units, comprises: comparing one or more of: (a) the one or more of the olefin feed sample properties, (b) the one or more paraffin feed sample properties, (c) the one or more the unit material sample properties, or (d) one or more properties of the one or more downstream materials, to one or more of: (i) material properties of a material database; (ii) one or more threshold values associated with operation of one or more of: (aa) the one or more first processing units, (bb) the alkylation unit, or (cc) the one or more second processing units; or (iii) one or more respective target properties of one or more of (aa) the one or more unit materials, or (bb) the one or more target properties of the one or more downstream materials.
Clause 5. The method of clause 1, wherein the prescriptively controlling comprises controlling one or more process parameters associated with the alkylation unit, the one or more process parameters comprising one or more of: (a) one or more olefin feed parameters associated with the olefin feed, the one or more olefin feed parameters comprising one or more of (i) a rate of supply of the olefin feed, (ii) a pressure associated with the olefin feed, or (iii) a temperature of the olefin feed; (b) one or more paraffin feed parameters associated with the paraffin feed, the one or more of the paraffin feed parameters comprising one or more of: (i) a rate of supply of the paraffin feed, (ii) a pressure associated with the paraffin feed, or (iii) a temperature of the paraffin feed; or (c) one or more catalyst parameters associated with the one or more catalysts, the one or more catalyst parameters comprising one or more of: (i) a rate of supply of the one or more catalysts, (ii) a pressure associate with the one or more catalysts, or (iii) a temperature of the one or more catalysts.
Clause 6. The method of clause 1, further comprising: analyzing, via one or more spectroscopic analyzers, a downstream material sample produced by the one or more second processing units, thereby to provide downstream material sample spectra; and predicting one or more downstream material sample properties associated with the downstream material sample based at least in part on the downstream material sample spectra.
Clause 7. The method of clause 1, wherein one or more of: (a) the analyzing of the olefin feed sample is performed on-line and in real-time; (b) the analyzing of the olefin feed sample is performed off-line in a laboratory setting; (c) the analyzing of the paraffin feed sample is performed on-line and in real-time; (d) the analyzing of the paraffin feed sample is performed off-line in a laboratory setting; (e) the analyzing of the unit material sample is performed on-line and in real-time; or (f) the analyzing of the unit material sample is performed off-line and in a laboratory setting.
Clause 8. The method of clause 1, wherein one or more of: (a) the predicting of the one or more olefin feed sample properties comprises predicting a boiling point associated with the olefin feed sample, and the method comprises controlling, based at least in part on the boiling point associated with the olefin feed sample, operation of one or more of: (i) the one or more first processing units, (ii) the alkylation unit, or the (iii) one or more second processing units; (b) the predicting of the one or more paraffin feed sample properties comprises predicting a boiling point associated with the paraffin sample, and the method comprises controlling, based at least in part on the boiling point associated with the paraffin feed sample, operation of one or more of: (i) the one or more first processing units, (ii) the alkylation unit, or the (iii) one or more second processing units; or (c) the predicting of the one or more corresponding unit materials comprises predicting a boiling point associated with the unit material sample, and the method comprises controlling, based at least in part on the boiling point associated with the unit material sample, operation of one or more of: (i) the one or more first processing units, (ii) the alkylation unit, or the (iii) one or more second processing units.
Clause 9. The method of clause 1, wherein: (a) the alkylation unit comprises a reactor positioned to receive the olefin feed and the paraffin feed; and (b) the prescriptively controlling comprises controlling one or more of: (i) content of the olefin feed supplied to the reactor; (ii) content of the paraffin feed supplied to the reactor; (iii) a temperature inside the reactor; (iv) a water content inside the reactor; (v) purity of the paraffin feed supplied to the reactor; or (vi) a ratio of the olefin feed to the paraffin feed supplied to the reactor.
Clause 10. The method of clause 9, wherein the controlling comprises controlling the temperature inside the reactor to be equal to or less than 50 degrees Celsius (C).
Clause 11. The method of clause 1, wherein one or more of: (a) the alkylation unit comprises a depropanizer, and the prescriptively controlling comprises controlling one or more of: (i) one or more operating parameters associated with the depropanizer; or (ii) content of the one or more unit materials produced by the depropanizer; (b) the alkylation unit comprises a deisobutanizer, and the prescriptively controlling comprises controlling one or more of: (i) one or more operating parameters associated with the deisobutanizer; or (ii) content of the one or more unit materials produced by the deisobutanizer; (c) the alkylation unit comprises a debutanizer, and the prescriptively controlling comprises controlling one or more of: (i) one or more operating parameters associated with the debutanizer; or (ii) content of the one or more unit materials produced by the debutanizer, or (d) the alkylation unit comprises an isostripper, and the prescriptively controlling comprises controlling one or more of: (i) one or more operating parameters associated with the isostripper; or (ii) content of the one or more unit materials produced by the isostripper.
Clause 12. The method of clause 11, wherein: (a) the alkylation unit comprises a depropanizer, and the one or more unit materials produced by the depropanizer comprise a propane product stream and a paraffin recycle stream; and (b) the prescriptively controlling comprises controlling a ratio of the propane product stream to the paraffin recycle stream produced by the depropanizer.
Clause 13. The method of clause 1, wherein: (a) the alkylation unit comprises a reactor and one or more acid settlers positioned to receive a feed from the reactor; and (b) the prescriptively controlling comprises controlling one or more of: (i) one or more operating parameters associated with the reactor; (ii) purity of one or more acid catalysts supplied to the reactor; (iii) a feed rate of one or more catalysts supplied to the reactor; (iv) water content inside the reactor; (v) content of unit materials associated with the feed from the reactor; (vi) one or more operating parameters associated with the one or more acid settlers; or (vii) content of unit materials produced by the one or more acid settlers.
Clause 14. The method of clause 1, wherein: (a) the unit materials comprise a paraffin recycle stream having a paraffin recycle purity; and (b) the prescriptively controlling comprises controlling, based at least in part on the paraffin recycle purity, a ratio of the olefin feed to the paraffin feed supplied to the alkylation unit.
Clause 15. The method of clause 1, wherein: (a) the one or more unit materials comprises an alkylate product for use in blending gasoline; and (b) the prescriptively controlling comprises controlling a ratio of the olefin feed to the paraffin feed supplied to the alkylation unit, thereby to control an octane level associated with the alkylate product produced by the alkylation unit.
Clause 16. The method of clause 1, wherein one or more of: (a) the olefin feed comprises one or more of butylenes, pentylenes, propylenes, or amylenes; (b) the paraffin feed comprises one or more of isobutane or isopentane; (c) the one or more catalysts comprise one or more of aluminum chloride, sulfuric acid, or hydrogen fluoride; or (d) the one or more unit materials comprise isooctane.
Clause 17. The method of clause 1, wherein the processing occurs at one of: (a) a temperature ranging from about 50 degrees Celsius (C) to about 280 degrees C. and at a pressure ranging from about 300 lbs. per square inch (psi) to about 1,000 psi; or (b) a temperature ranging from about 1 degree C. to about 40 degrees C. and at a pressure ranging from about 14 psi to about 150 psi.
Clause 18. The method of clause 1, wherein the one or more second processing units comprise at least a portion of a gasoline blending operation.
Clause 19. The method of clause 1, wherein one or more of the first spectroscopic analyzer, the second spectroscopic analyzer, or the third spectroscopic analyzer, comprises one of a near-infrared spectroscopic analyzer, a mid-infrared spectroscopic analyzer, a combination of a near-infrared spectroscopic analyzer and a mid-infrared spectroscopic analyzer, a Raman spectroscopic analyzer, or a nuclear magnetic resonance spectroscopic analyzer.
Clause 20. The method of clause 1, further comprising one or more of: (a) prescriptively controlling one or more of a temperature of the olefin feed sample, a temperature of the paraffin feed, or a temperature of the unit material sample, thereby to substantially maintain one or more of a temperature of the olefin feed sample, the paraffin feed sample, or the unit material sample, respectively, within a respective temperature range; or (b) applying one or more temperature correction factors to one or more of: (i) the olefin feed sample spectra; (ii) the paraffin feed sample spectra; or (iii) the unit material sample spectra.
Clause 21. The method of clause 1, wherein the one or more of (a) the one or more olefin feed sample properties, (b) the one or more paraffin feed sample properties, (c) the one or more unit material sample properties, or (d) one or more downstream material properties of the one or more downstream materials, comprise a content ratio indicative of relative amounts of one or more hydrocarbon classes present in one or more of (a) the olefin feed sample, (b) the paraffin feed sample, (c) the unit material sample, or (d) a downstream material sample, respectively.
Clause 22. The method of clause 1, further comprising one or more of: (a) conditioning the olefin feed sample, prior to analyzing the olefin feed, to one or more of filter the olefin feed sample, change a temperature of the olefin feed sample, or degas the olefin feed sample; (b) conditioning the paraffin feed sample, prior to analyzing the paraffin feed, to one or more of filter the paraffin feed sample, change a temperature of the paraffin feed sample, or degas the paraffin feed sample; (c) conditioning the unit material sample, prior to analyzing the unit material sample, to one or more of filter the unit material sample, change a temperature of the unit material sample, or degas the unit material sample; or (d) conditioning a downstream material sample of the one or more downstream materials, prior to analyzing the downstream material sample, to one or more of filter the downstream t material sample, change a temperature of the downstream material sample, or degas the downstream material sample.
Clause 23. The method of clause 1, further comprising analyzing, via a second spectroscopic analyzer, one or more of: (a) the paraffin feed sample, (b) the unit material sample, or (c) a downstream material sample of the one or more downstream materials, the first spectroscopic analyzer and the second spectroscopic analyzer being calibrated to generate standardized spectral responses.
Clause 24. The method of clause 1, further comprising analyzing, via a second spectroscopic analyzer, one or more of: (a) the paraffin feed sample, (b) the unit material sample, or (c) a downstream material sample of the one or more downstream materials, wherein one or more of the first spectroscopic analyzer or the second spectroscopic analyzer, is linked to a signal processing device configured to perform numerical treatment of one or more of the olefin sample spectra, the paraffin sample spectra, or the unit material sample spectra, the numerical treatment comprising one or more of a partial least squares regression, a principal component regression, a Gauss-Jordan row reduction, or a multiple linear regression.
Clause 25. The method of clause 1, wherein the paraffin feed comprises one or more of isobutane from the operations of an FCC unit, isobutane from the operations of a hydrocracking unit, isobutane from the distillation of crude oil in a crude unit, or isobutane from the operations of a reformer unit.
Clause 26. The method of clause 1, wherein the method further comprises: determining one or more properties of the one or more downstream materials, the one or more downstream materials comprising blended gasoline including at least a portion of the one or more unit materials; wherein the one or more properties comprise one or more of a Research Octane Number (RON), a Motor Octane Number (MON), Anti-Knock Index (AKI), a total aromatics content, a total butane content, an amount of isobutane, an amount of n-isobutane, a total olefins content, an amount of gasoline, an amount of propane, an amount of pentane, an amount of hydrogen sulfide, an amount of sulfur, an amount of water content, an aniline point, an amount of propane, an amount of benzene, an amount of toluene, an amount of xylene, total Benzene Toluene Xylene (BTX) content, total Benzene Toluene Ethylbenzene Xylene (BTEX) content, a vapor pressure, distillation properties/parameters, a drivability index, a density, a specific gravity, an American Petroleum Institute (API) gravity, an amount of alcohol, or an Ethanol (EtOH) kick prediction.
Clause 27. The method of clause 1, wherein at least one of the first spectroscopic analyzer, the second spectroscopic analyzer, or the third spectroscopic analyzer is calibrated using calibration data derived from one or more of: (a) a set of known reference fuels; (b) a library of known spectral data; or (c) an analytical model of the alkylation process.
Clause 28. The method of clause 1, wherein the one or more unit materials comprises one or more of a catalyst recycle product and a paraffin recycle product, and the unit material sample properties comprise at least one of: (a) a concentration of the catalyst recycle product; (b) a known weight percent of water in the catalyst recycle product; (c) a known weight percent of acid oil in the catalyst recycle product; or (d) a known concentrations of a paraffin recycle product in the paraffin recycle stream.
Clause 29. An alkylation process control assembly to enhance control of an alkylation process associated with a refining operation, the alkylation process control assembly comprising: (a) a first spectroscopic analyzer positioned to: (i) receive an olefin feed sample of an olefin feed supplied to an alkylation unit associated with the alkylation process, the olefin feed having one or more olefin feed properties; and (ii) analyze the olefin feed sample to provide olefin feed sample spectra; (b) a second spectroscopic analyzer positioned to: (i) receive a paraffin feed sample of a paraffin feed supplied to the alkylation unit associated with the alkylation process, the paraffin feed having one or more paraffin feed properties; and (ii) analyze the paraffin feed sample to provide paraffin feed sample spectra; (c) a third spectroscopic analyzer positioned to: (i) receive a unit material sample of one more unit materials produced by the alkylation unit associated with the alkylation process, the one or more unit materials comprising one or more of intermediate materials or unit product materials, the first spectroscopic analyzer, the second spectroscopic analyzer, and the third spectroscopic analyzer being calibrated to generate standardized spectral responses; and (ii) analyze the unit material sample to provide unit material sample spectra; and (d) an alkylation process controller in communication with the first spectroscopic analyzer, the second spectroscopic analyzer, and the third spectroscopic analyzer, the alkylation process controller configured to: (i) predict one or more olefin feed sample properties associated with the olefin feed sample based at least in part on the olefin feed sample spectra; (ii) predict one or more paraffin feed sample properties associated with the paraffin feed sample based at least in part on the paraffin feed sample spectra; (iii) predict one or more unit material sample properties associated with the unit material sample based at least in part on the unit material sample spectra; and (iv) prescriptively control, during the alkylation process, based at least in part on the one or more olefin feed sample properties, the one or more paraffin feed sample properties, and the one or more unit material sample properties, one or more of: (aa) one or more olefin feed parameters associated with the olefin feed supplied to the alkylation unit; (bb) the one or more olefin feed properties associated with the olefin feed supplied to alkylation unit; (cc) one or more paraffin feed parameters associated with the paraffin feed supplied to the alkylation unit; (dd) the one or more paraffin feed properties associated with the paraffin feed supplied to the alkylation unit; (ee) one or more unit materials properties associated with the one or more unit materials; (ff) operation of the alkylation unit; or (gg) operation of one or more second processing units positioned downstream relative to the alkylation unit, so that the prescriptively controlling during the alkylation process causes the alkylation process to produce one or more of: (aa) one or more intermediate materials each having one or more properties within a selected range of one or more target properties of the one or more intermediate materials; (bb) one or more unit materials each having one or more properties within a selected range of one or more target properties of the one or more unit materials; or (cc) one or more downstream materials each having one or more properties within a selected range of one or more target properties of the one or more downstream materials, thereby to cause the refining process to achieve material outputs that more accurately and responsively converge on one or more target properties.
Clause 30. The alkylation process control assembly of clause 29, wherein the alkylation process controller is configured to control one or more of: (a) content of the olefin feed supplied to the alkylation unit; (b) content of the paraffin feed supplied to the alkylation unit; (c) content of one or more catalysts processed by the alkylation unit; (d) content of the intermediate materials produced by the alkylation unit; (e) content of the unit product materials produced by the alkylation unit; (f) the operation of the alkylation unit; or (h) the operation of one or more second processing units downstream of the alkylation unit.
Clause 31. The alkylation process control assembly of clause 29, wherein one or more of: (a) the analyze the olefin feed sample is performed on-line and in real-time; (b) the analyze the olefin feed sample is performed off-line in a laboratory setting; (c) the analyze the paraffin feed sample is performed on-line and in real-time; (d) the analyze the paraffin feed sample is performed off-line in a laboratory setting; (e) the analyze the unit material sample is performed on-line and in real-time; or (f) the analyze the unit material sample is performed off-line in a laboratory setting.
Clause 32. The alkylation process control assembly of clause 29, wherein the alkylation process controller is configured to improve an accuracy of one or more of: (a) the one or more olefin feed sample properties associated with the olefin feed sample; (b) the one or more paraffin feed sample properties associated with the paraffin feed sample; (c) the one or more unit material sample properties associated with the unit material sample; (d) content of the olefin feed supplied to the alkylation unit; (e) content of the paraffin feed supplied to the alkylation unit; (f) content of one or more catalysts processed by the alkylation unit; (g) content of the one or more intermediate materials produced by the alkylation unit; (h) content of the unit product materials produced by the alkylation unit; or (i) content of the downstream materials produced by one or more of the second processing units.
Clause 33. The alkylation process control assembly of clause 29, wherein the alkylation process controller further comprises a sample conditioning assembly positioned to one or more of: (i) condition the olefin feed sample, prior to being supplied to the first spectroscopic analyzer, to one or more of filter the olefin feed sample, change a temperature of the olefin feed sample, or degas the olefin feed sample; (ii) condition the paraffin feed sample, prior to being supplied to the second spectroscopic analyzer, to one or more of filter the paraffin feed sample, change a temperature of the paraffin feed sample, or degas the paraffin feed sample; or (iii) condition the unit material sample, prior to being supplied to the third spectroscopic analyzer, to one or more of filter the unit material sample, change a temperature of the unit material sample, or degas the unit material sample.
Clause 34. The alkylation process control assembly of clause 29, wherein the alkylation process controller is configured to control one or more process parameters, the one or more process parameters comprising one or more of: (a) one or more olefin feed parameters associated with the olefin feed, the one or more olefin feed parameters comprising one or more of (i) a rate of supply of the olefin feed, (ii) a pressure associated with the olefin feed, or (iii) a temperature of the olefin feed; (b) one or more paraffin feed parameters associated with the paraffin feed, the one or more of the paraffin feed parameters comprising one or more of: (i) a rate of supply of the paraffin feed, (ii) a pressure associated with the paraffin feed, or (iii) a temperature of the paraffin feed; or (c) one or more catalyst parameters associated with one or more catalysts processed by alkylation unit, the one or more catalyst parameters comprising one or more of: (i) a rate of supply of the one or more catalysts, (ii) a pressure associate with the one or more catalysts, or (iii) a temperature of the one or more catalysts.
Clause 35. The alkylation process control assembly of clause 29, wherein the alkylation process controller is configured to control one or more process parameters, the one or more process parameters comprising one or more of: (a) a purity of one or more catalysts processed by the alkylation unit; (b) a feed rate of one or more catalysts processed by the alkylation unit; (c) a purity of the paraffin feed; or (d) a ratio of the olefin feed to the paraffin feed.
Clause 36. The alkylation process control assembly of clause 29, further comprising: determining one or more downstream properties associated with the one or more downstream materials, the one or more downstream materials comprising blended gasoline including at least a portion of the one or more unit materials, the one or more downstream properties comprising one or more of: a Research Octane Number (RON), a Motor Octane Number (MON), Anti-Knock Index (AKI), a total aromatics content, a total butane content, an amount of isobutane, an amount of n-isobutane, a total olefins content, an amount of gasoline, an amount of propane, an amount of pentane, an amount of hydrogen sulfide, an amount of sulfur, an amount of water content, an aniline point, an amount of propane, an amount of benzene, an amount of toluene, an amount of xylene, total Benzene Toluene Xylene (BTX) content, total Benzene Toluene Ethylbenzene Xylene (BTEX) content, a vapor pressure, distillation properties/parameters, a drivability index, a density, a specific gravity, an American Petroleum Institute (API) gravity, an amount of alcohol, or an Ethanol (EtOH) kick prediction.
Clause 37. The alkylation process control assembly of clause 29, wherein the prescriptively control of one or more of: (a) the alkylation unit, or (b) the one or more second processing units, comprises: comparing one or more of: (a) the one or more olefin feed sample properties, (b) the one or more paraffin feed sample properties, or (c) the one or more unit material sample properties, to one or more of: (i) material properties of a material database; (ii) one or more threshold values associated with operation of one or more of: (aa) the alkylation unit, or (bb) the one or more second processing units; or (iii) target properties.
Clause 38. The alkylation process control assembly of clause 37, wherein the target properties comprise target content associated with one or more of: (a) a content of the olefin feed supplied to the alkylation unit; (b) a content of the paraffin feed supplied to the alkylation unit; (c) a content of one or more catalysts supplied to the alkylation unit; (d) content of the intermediate materials produced by the alkylation unit; (e) content of the unit product materials produced by the alkylation unit; or (f) content of the downstream materials produced by one or more of the second processing units.
Clause 39. The alkylation process control assembly of clause 29, wherein the one or more olefin feed sample properties, the one or more paraffin feed sample properties, and the one or more unit material sample properties comprise a content ratio indicative of relative amounts of one or more hydrocarbon classes present in one or more of (a) the olefin feed sample, (b) the paraffin feed sample, or (c) the unit material sample.
Clause 40. The alkylation process control assembly of clause 29, wherein the one or more unit material sample properties comprise one or more of: a Research Octane Number (RON), a Motor Octane Number (MON), Anti-Knock Index (AKI), a total aromatics content, a total butane content, an amount of isobutane, an amount of n-isobutane, a total olefins content, an amount of gasoline, an amount of propane, an amount of pentane, an amount of hydrogen sulfide, an amount of sulfur, an amount of water content, an aniline point, an amount of propane, an amount of benzene, an amount of toluene, an amount of xylene, total Benzene Toluene Xylene (BTX) content, total Benzene Toluene Ethylbenzene Xylene (BTEX) content, a vapor pressure, distillation properties/parameters, a drivability index, a density, a specific gravity, an American Petroleum Institute (API) gravity, an amount of alcohol, or an Ethanol (EtOH) kick prediction.
Clause 41. The alkylation process control assembly of clause 29, wherein the one or more unit material sample properties comprise one or more of: an amount of acid oil, an amount of water, an amount of acid-soluble hydrocarbons, or an amount of organic contaminants.
Clause 42. The alkylation process control assembly of clause 29, wherein the one or more intermediate properties or unit material sample properties comprise at least one of: a catalyst concentration of the catalyst recycle product, a known weight percent of water in the catalyst recycle product, or a known weight percent of acid oil in the catalyst recycle product.
Clause 43. The alkylation process control assembly of clause 29, wherein the alkylation process controller is configured to control one or more operating parameters associated with the alkylation unit against operating constraints of the alkylation unit.
Clause 44. The alkylation process control assembly of clause 29, wherein: (a) the alkylation unit comprises a depropanizer, and the prescriptive control comprises controlling one or more of: (i) one or more operating parameters associated with the depropanizer; or (ii) content of the one or more unit materials produced by the depropanizer; (b) the alkylation unit comprises a deisobutanizer, and the prescriptive control comprises controlling one or more of: (i) one or more operating parameters associated with the deisobutanizer; or (ii) content of the one or more unit materials produced by the deisobutanizer; (c) the alkylation unit comprises a debutanizer, and the prescriptive control comprises controlling one or more of: (i) one or more operating parameters associated with the debutanizer; or (ii) content of the one or more unit materials produced by the debutanizer; or (d) the alkylation unit comprises an isostripper, and the prescriptively controlling comprises controlling one or more of: (i) one or more operating parameters associated with the isostripper; or (ii) content of the one or more unit materials produced by the isostripper.
Clause 45. The alkylation process control assembly of clause 44, wherein the alkylation process controller is configured to control one or more of: (a) the one or more operating parameters associated with the depropanizer against operating constraints of the depropanizer; (b) the one or more operating parameters associated with the deisobutanizer against operating constraints of the deisobutanizer; (c) the one or more operating parameters associated with the debutanizer against operating constraints of the debutanizer; or (d) the one or more operating parameters associated with the isostripper against operating constraints of the isostripper.
Clause 46. The alkylation process control assembly of clause 29, wherein the alkylation process controller is configured to control, prior to the analyzing, one or more of: (a) conditioning the olefin feed samples to provide a conditioned olefin feed sample; (b) conditioning the paraffin feed sample to provide a conditioned paraffin feed sample; or (c) conditioning the unit material sample to provide a conditioned unit material sample.
Clause 47. The alkylation process control assembly of clause 29, wherein the alkylation process controller is configured to numerically treat one or more of: a spectral response of the first spectroscopic analyzer, a spectral response of the second spectroscopic analyzer, or a spectral response of the third spectroscopic analyzer.
Clause 48. The alkylation process control assembly of clause 29, wherein the one or more intermediate properties comprises a known concentration of a paraffin recycle product in a paraffin recycle stream.
Clause 49. The alkylation process control assembly of clause 29, wherein one or more of the first spectroscopic analyzer, the second spectroscopic analyzer, or the third spectroscopic analyzer comprises one of a near-infrared spectroscopic analyzer, a mid-infrared spectroscopic analyzer, a combination of one or more near-infrared spectroscopic analyzers and a mid-infrared spectroscopic analyzers, a Raman spectroscopic analyzer, or a nuclear magnetic resonance spectroscopic analyzer.
Clause 50. The alkylation process control assembly of clause 29, wherein one or more of: (a) the alkylation unit comprises a depropanizer, and the prescriptively controlling comprises controlling one or more of: (i) one or more operating parameters associated with the depropanizer; or (ii) content of the one or more unit materials produced by the depropanizer; (b) the alkylation unit comprises a deisobutanizer, and the prescriptively controlling comprises controlling one or more of: (i) one or more operating parameters associated with the deisobutanizer; or (ii) content of the one or more unit materials produced by the deisobutanizer; (c) the alkylation unit comprises a debutanizer, and the prescriptively controlling comprises controlling one or more of: (i) one or more operating parameters associated with the debutanizer; or (ii) content of the one or more unit materials produced by the debutanizer; or (d) the alkylation unit comprises an isostripper, and the prescriptively controlling comprises controlling one or more of: (i) one or more operating parameters associated with the isostripper; or (ii) content of the one or more unit materials produced by the isostripper.
Clause 51. An alkylation process controller to enhance control of an alkylation process associated with a refining operation, the alkylation process controller being in communication with one or more spectroscopic analyzers and one or more alkylation processing units, the alkylation process controller being configured to: predict one or more olefin feed sample properties associated with an olefin feed sample based at least in part on olefin feed sample spectra generated by the one or more spectroscopic analyzers; predict one or more paraffin feed sample properties associated with a paraffin feed sample based at least in part on paraffin feed sample spectra generated by the one or more spectroscopic analyzers; predict one or more unit material sample properties associated with a unit material sample based at least in part on unit material sample spectra generated by the one or more spectroscopic analyzers, the one or more unit material sample properties being associated with one or more unit materials produced by the one or more alkylation processing units, and the one or more unit materials comprising one or more of intermediate materials or unit product materials; and prescriptively control, during the alkylation process, based at least in part on the one or more olefin feed sample properties, the one or more paraffin feed sample properties, and the one or more unit material sample properties, one or more of: (a) the one or more olefin feed properties associated with the olefin feed supplied to the one or more alkylation processing units; (b) the one or more paraffin feed properties associated with the paraffin feed supplied to the one or more alkylation processing units; (c) one or more catalyst feed properties associated with a catalyst feed supplied to the one or more alkylation processing units; (d) one or more intermediate properties associated with the intermediate materials produced by one or more of the alkylation processing units; (e) operation of the one or more alkylation processing units; (f) one or more unit materials properties associated with the one or more unit materials produced by the one or more alkylation processing units; or (g) operation of one or more second processing units positioned downstream relative to the one or more alkylation processing units, so that the prescriptively controlling causes the refining process to produce one or more of: (i) the one or more intermediate materials each having one or more properties within a selected range of one or more target properties of the one or more intermediate materials; (ii) the one or more unit materials each having one or more properties within a selected range of one or more target properties of the one or more unit materials; or (iii) one or more downstream materials produced by the second processing units each having one or more properties within a selected range of one or more target properties of the one or more downstream materials, thereby to cause the refining process to achieve material outputs that more accurately and responsively converge on one or more of the target properties.
Clause 52. The alkylation process controller of clause 51, wherein the alkylation process controller is configured to prescriptively control, during the refining process, one or more process parameters of the one or more alkylation processing units against operating constraints associated with the one or more alkylation processing units.
Clause 53. The alkylation process controller of clause 51, wherein the prescriptively controlling comprises controlling one or more process parameters, the one or more process parameters comprising one or more of: (a) one or more olefin feed parameters; (b) a rate of supply of the olefin feed supplied to the one or more alkylation processing units; (c) a pressure of the olefin feed supplied to the one or more alkylation processing units; (d) a preheating temperature of the olefin feed supplied to the one or more first processing units; (e) one or more paraffin feed parameters; (f) a rate of supply of the paraffin feed suppled to the one or more alkylation processing units; (g) a pressure of the paraffin feed supplied to the one or more alkylation processing units; (h) a preheating temperature of the paraffin feed supplied to the one or more alkylation processing units; (i) one or more catalyst feed parameters of the catalyst feed suppled to the one or more alkylation processing units; (j) a rate of supply of the catalyst feed to the one or more alkylation processing units; (k) a pressure of the catalyst feed supplied to the one or more alkylation processing units; (1) a preheating temperature of the catalyst feed supplied to the one or more alkylation processing units; (m) one or more unit materials parameters; (n) a rate of supply of the one or more unit materials to the one or more second processing units; (o) a pressure of the one or more unit materials supplied to the one or more second processing units; (p) a preheating temperature of the one or more unit materials supplied to the one or more second processing units; (q) a temperature in the one or more alkylation processing units; (r) a pressure in the one or more alkylation processing units; (s) a temperature in the one or more second processing units; or (t) a pressure in the one or more second processing units.
Clause 54. The alkylation process controller of clause 51, wherein one or more of: (a) the properties associated with one or more of the olefin feed, the paraffin feed, the intermediate materials, and the unit product materials comprising one or more of a Research Octane Number (RON), a Motor Octane Number (MON), Anti-Knock Index (AKI), a total aromatics content, a total butane content, an amount of isobutane, an amount of n-isobutane, a total olefins content, an amount of gasoline, an amount of propane, an amount of pentane, an amount of hydrogen sulfide, an amount of sulfur, an amount of water content, an aniline point, an amount of propane, an amount of benzene, an amount of toluene, an amount of xylene, total Benzene Toluene Xylene (BTX) content, total Benzene Toluene Ethylbenzene Xylene (BTEX) content, a vapor pressure, distillation properties/parameters, a drivability index, a density, a specific gravity, an American Petroleum Institute (API) gravity, an amount of alcohol, or an Ethanol (EtOH) kick prediction; or (b) the one or more olefin feed sample properties, the one or more paraffin feed sample properties, and the one or more unit material sample properties comprise a content ratio indicative of relative amounts of one or more hydrocarbon classes present in one or more of the olefin feed sample, the paraffin feed sample, or the unit material sample.
Clause 55. A method for enhancing control of an alkylation process associated with a refining operation, the method comprising: (a) predicting one or more target properties of one or more unit materials produced by the alkylation process; (b) predicting, during a first time cycle a first portion of one or more of: (i) one or more first olefin feed sample properties associated with a first olefin feed sample based at least in part on first olefin feed sample spectra generated by one or more spectroscopic analyzers; (ii) one or more first paraffin feed sample properties associated with a first paraffin feed sample based at least in part on first paraffin feed sample spectra generated by the one or more spectroscopic analyzers; or (iii) one or more first unit material sample properties associated with a first unit material sample based at least in part on first unit material sample spectra generated by the one or more spectroscopic analyzers, the unit material sample properties being associated with the one or more unit materials produced by the alkylation process and the one or more unit materials comprising one or more of intermediate materials or unit product materials; (c) determining, during the first time cycle, a first set of differences between (i) the one or more target properties of the one or more unit materials produced by the alkylation process and (ii) the one or more first unit material sample properties associated with the first unit material sample; (d) generating, during the first time cycle, based at least in part on the first set of differences, one or more first control signals indicative of one or more process parameters associated with the alkylation process; (e) controlling, during the first time cycle, based at least in part on the one or more first control signals, one or more of: (i) one or more olefin feed properties associated with the olefin feed; (ii) one or more paraffin feed properties associated with the paraffin feed; (iii) one or more catalyst feed properties associated with a catalyst feed of the alkylation process; (iv) one or more intermediate properties associated with the intermediate materials produced by the alkylation process; (v) the one or more process parameters associated with operation of the alkylation process; (vi) one or more process parameters associated with operation of one or more first processing units positioned upstream relative to the alkylation process; or (vii) one or more process parameters associated with operation of one or more second processing units positioned downstream relative to the alkylation process; so that the controlling causes the alkylation process to achieve material outputs that more accurately and responsively converge on the one or more target properties of the one or more unit materials produced by the alkylation process; (f) predicting, during a second time cycle after the first time cycle, one or more of: (i) one or more second olefin feed sample properties associated with a second olefin feed sample based at least in part on second olefin feed sample spectra generated by the one or more spectroscopic analyzers; (ii) one or more second paraffin feed sample properties associated with a second paraffin feed sample based at least in part on second paraffin feed sample spectra generated by the one or more spectroscopic analyzers; or (iii) one or more second unit material sample properties associated with a second unit material sample based at least in part on second unit material sample spectra generated by the one or more spectroscopic analyzers; (g) determining, during the second time cycle, a second set of differences between (i) the one or more target properties of the one or more unit materials produced by the alkylation process and (ii) the one or more second unit material sample properties associated with the second unit material sample; (h) generating, during the second time cycle, based at least in part on the second set of differences, one or more second control signals indicative of one or more process parameters associated with the alkylation process; (i) controlling, during the second time cycle, based at least in part on the one or more second control signals, one or more of: (i) the one or more olefin feed properties associated with the olefin feed; (ii) the one or more paraffin feed properties associated with the paraffin feed; (iii) the one or more catalyst feed properties associated with the catalyst feed; (iv) the one or more intermediate properties associated with the intermediate materials produced by the alkylation process; (v) the one or more process parameters associated with operation of the alkylation process; (vi) the one or more process parameters associated with operation of one or more first processing units positioned upstream relative to the alkylation process; or (vii) the one or more process parameters associated with operation of one or more second processing units positioned downstream relative to the alkylation process; so that the controlling is iterative between the one or more first control signals, the one or more second control signals, and one or more nth control signals, thereby to cause the alkylation process to achieve material outputs that more accurately and responsively converge on the one or more target properties of the unit materials produced by the alkylation process.
Clause 56. The method of clause 55, further comprising: (a) filtering one or more of the first olefin feed sample, the first paraffin feed sample, or the first unit material sample during the first time cycle; the second olefin feed sample, the second paraffin feed sample, or the second unit material sample during the second time cycle; or an nth olefin feed sample, an nth paraffin feed sample, or an nth unit material sample during an nth time cycle to reduce one or more of water, particulates, or other contaminates, thereby to provide a filtered portion of the sample stream during a corresponding one or more of the first time cycle, the second time cycle, or the nth time cycle; and (b) controlling a temperature of the filtered portion of the sample stream to be within a preselected temperature range, thereby to provide a conditioned portion of the sample stream.
Clause 57. The method of clause 55, wherein one or more of: (a) the olefin feed comprises one or more of butylenes, pentylenes, propylenes, or amylenes; (b) the paraffin feed comprises one or more of isobutane or isopentane; (c) the one or more catalysts comprise one or more of aluminum chloride, sulfuric acid, or hydrogen fluoride; or (d) the one or more unit materials comprise isooctane.
Clause 58. The method of clause 55, wherein the one or more intermediate materials comprise one or more of: (a) a catalyst recycle product; (b) a paraffin recycle product; or (c) an alkylation product from an alkylation product stream.
Clause 59. The method of clause 58, wherein the one or more target properties of the one or more unit materials comprises at least one of: (a) acid concentration of the catalyst recycle product; (b) content of water in the catalyst recycle product; (c) content of acid oil in the catalyst recycle product; or (d) concentrations of an isobutane recycle product in the paraffin recycle stream.
Clause 60. The method of clause 55, wherein the one or more spectroscopic analyzers are calibrated using calibration data derived from one or more of: (a) a set of known reference fuels; (b) a library of known spectral data; or (c) an analytical model of the alkylation process.
Clause 61. The method of clause 55, wherein the one or more spectroscopic analyzers comprises one or more of: (a) one or more near-infrared (NIR) spectroscopic analyzers; (b) one or more mid-infrared (mid-IR) spectroscopic analyzers; (c) one or more combined NIR and mid-IR spectroscopic analyzers; (d) one or more Raman spectroscopic analyzers; or (e) one or more nuclear magnetic resonance (NMR) spectroscopic analyzers.
Clause 62. The method of clause 55, wherein each of the first plurality of properties and the second plurality of properties of the first portion of the sample stream and the second portion of the sample stream, respectively, comprises one or more of: Research Octane Number (RON), Motor Octane Number (MON), Anti-Knock Index (AKI), total aromatics content, total olefins content, benzene content, toluene content, xylenes content, total Benzene Toluene Xylene (BTX) content, total Benzene Toluene Ethylbenzene Xylene (BTEX) content, vapor pressure, distillation properties/parameters, drivability index, density, specific gravity, American Petroleum Institute (API) gravity, alcohol contents, or Ethanol (EtOH) kick predictions.
Clause 63. The method of clause 55, the method further comprising: controlling the one or more process parameters associated with the alkylation process until: (1) a difference between the one or more target properties of the unit materials and the one or more unit material sample properties associated with the unit material sample approaches zero, and (2) the one or more target properties of the unit materials are within a predetermined range of preselected target properties.
Clause 64. The method of clause 55, wherein the one or more unit product materials comprises an alkylate product for use in blending gasoline, and the controlling comprises controlling one or more of: (a) a ratio of the olefin feed to the paraffin feed supplied to the alkylation process, thereby to control an octane level associated with the alkylate product; or (b) a target composition of the alkylate product.
Clause 65. An alkylation processing assembly for performing an alkylation process associated with a refining operation, the alkylation processing assembly comprising: (a) one or more alkylation processing units associated with the refining operation, the one or more alkylation processing units including one or more of a reactor, an acid settler, an isostripper, a depropanizer, a deisobutanizer, or a debutanizer; (b) a first spectroscopic analyzer positioned to: (i) receive an olefin feed sample of an olefin feed positioned to be supplied to the one or more alkylation processing units, the olefin feed having one or more olefin feed properties; and (ii) analyze the olefin feed sample to provide olefin feed sample spectra; (c) a second spectroscopic analyzer positioned to: (i) receive a paraffin feed sample of a paraffin feed positioned to be supplied to the one or more alkylation processing units, the paraffin feed having one or more paraffin feed properties; and (ii) analyze the paraffin feed sample to provide paraffin feed sample spectra; (d) a third spectroscopic analyzer positioned to: (i) receive a unit material sample of one more unit materials produced by the one or more alkylation processing units, the one or more unit materials comprising one or more of intermediate materials or unit product materials, the first spectroscopic analyzer, the second spectroscopic analyzer, and the third spectroscopic analyzer being calibrated to generate standardized spectral responses; and (ii) analyze the unit material sample to provide unit material sample spectra; and (f) an alkylation process controller in communication with the first spectroscopic analyzer, the second spectroscopic analyzer, and the third spectroscopic analyzer, the alkylation process controller configured to: (i) predict one or more olefin feed sample properties associated with the olefin feed sample based at least in part on the olefin feed sample spectra; (ii) predict one or more paraffin feed sample properties associated with the paraffin feed sample based at least in part on the paraffin feed sample spectra; (iii) predict one or more unit material sample properties associated with the unit material sample based at least in part on the unit material sample spectra; and (iv) prescriptively control, during the alkylation process, based at least in part on the one or more olefin feed sample properties, the one or more paraffin feed sample properties, and the one or more unit material sample properties, one or more of: (aa) one or more olefin feed parameters associated with the olefin feed supplied to the one or more alkylation processing units; (bb) the one or more olefin feed properties associated with the olefin feed supplied to the one or more alkylation processing units; (cc) one or more paraffin feed parameters associated with the paraffin feed supplied to the one or more alkylation processing units; (dd) the one or more paraffin feed properties associated with the paraffin feed supplied to the one or more alkylation processing units; (ee) one or more intermediate properties associated with the intermediate materials produced by the one or more alkylation processing units; (ff) one or more unit materials properties associated with the one or more unit materials; (gg) operation of the one or more alkylation processing units; or (hh) operation of one or more second processing units positioned downstream relative to the one or more alkylation processing units, so that the prescriptively controlling during the refining process causes the refining process to produce one or more of: (aa) one or more intermediate materials each having one or more properties within a selected range of one or more target properties of the one or more intermediate materials; (bb) one or more unit materials each having one or more properties within a selected range of one or more target properties of the one or more unit materials; or (cc) one or more downstream materials each having one or more properties within a selected range of one or more target properties of the one or more downstream materials, thereby to cause the alkylation process to achieve material outputs that more accurately and responsively converge on one or more of the target properties.
Clause 66. The alkylation processing assembly of clause 65, the alkylation processing assembly further comprising a sample conditioning assembly positioned to one or more of: (i) condition the olefin feed sample, prior to being supplied to the first spectroscopic analyzer, to one or more of filter the olefin feed sample, change a temperature of the olefin feed sample, dilute in solvent the olefin feed sample, or degas the olefin feed sample; (ii) condition the paraffin feed sample, prior to being supplied to the second spectroscopic analyzer, to one or more of filter the paraffin feed sample, change a temperature of the paraffin feed sample, dilute in solvent the paraffin feed sample, or degas the paraffin feed sample; or (iii) condition the unit material sample, prior to being supplied to the third spectroscopic analyzer, to one or more of filter the unit material sample, change a temperature of the unit material sample, dilute in solvent the unit material sample, or degas the unit material sample.
Clause 67. The alkylation processing assembly of clause 65, wherein: one or more of the olefin feed or the paraffin feed comprises a blended hydrocarbon feed including a plurality of hydrocarbon feeds from respective hydrocarbon feed flows; and the alkylation process controller is configured to control feed ratios of the respective hydrocarbon feed flows.
Clause 68. The alkylation processing assembly of clause 65, wherein the alkylation process controller is configured to control one or more of: (a) content of the olefin feed supplied to the one or more alkylation processing units; (b) content of the paraffin feed supplied to the one or more alkylation processing units; (c) content of one or more catalyst feeds processed by the one or more alkylation processing units; (d) operation of the one or more alkylation processing units; (e) content of the intermediate materials produced by the one or more alkylation processing units; or (f) content of the unit product materials produced by the one or more alkylation processing units.
Clause 69. The alkylation processing assembly of clause 65, wherein the alkylation process controller is further configured to control the one or more properties of the one or more downstream materials via control of one or more of: (a) one or more properties of the unit materials produced by the one or more alkylation processing units; (b) operation of the one or more alkylation processing units; or (c) one or more process parameters of the one or more second processing units positioned downstream relative to the one or more alkylation processing units.
Clause 70. The alkylation processing assembly of clause 65, wherein the alkylation process controller is configured to operate a prescriptive analytical model configured to improve an accuracy of one or more of: (a) the one or more olefin feed properties; (b) the one or more paraffin feed properties; (c) content of the one or more of the unit materials; (d) a purity of one or more catalysts processed by the one or more alkylation processing units; or (e) downstream materials produced by the one or more second processing units.
Clause 71. The alkylation processing assembly of clause 65, wherein the alkylation process controller is configured to one or more of: (a) control one or more of the temperature of the olefin feed sample, the temperature of the paraffin feed sample, or the temperature of the unit material sample, thereby to substantially maintain the one or more of the temperature of the olefin feed sample, the temperature of the paraffin feed sample, or the temperature of the unit material sample within a respective temperature range; or (b) apply one or more temperature correction factors to one or more of: (i) the olefin feed sample spectra; (ii) the paraffin feed sample spectra; or (iii) the unit material sample spectra.
Clause 72. The alkylation processing assembly of clause 65, wherein the alkylation process controller is configured to control one or more process parameters associated with the alkylation process, the one or more process parameters comprising one or more of: (a) one or more olefin feed parameters; (b) a rate of supply of the olefin feed supplied to the one or more alkylation processing units; (c) a pressure of the olefin feed supplied to the one or more alkylation processing units; (d) a preheating temperature of the olefin feed supplied to the one or more alkylation processing units; (e) one or more paraffin feed parameters; (f) a rate of supply of the paraffin feed suppled to the one or more alkylation processing units; (g) a pressure of the paraffin feed supplied to the one or more alkylation processing units; (h) a preheating temperature of the paraffin feed supplied to the one or more alkylation processing units; (i) one or more catalyst feed parameters of one or more catalyst feeds suppled to the one or more alkylation processing units; (j) a rate of supply of the one or more catalyst feeds to the one or more alkylation processing units; (k) a pressure of the one or more catalyst feeds supplied to the one or more alkylation processing units; (1) a preheating temperature of the one or more catalyst feeds supplied to the one or more alkylation processing units; (m) one or more unit materials parameters; (n) a rate of supply of the one or more unit materials to the one or more second processing units; (o) a pressure of the one or more unit materials supplied to the one or more second processing units; (p) a preheating temperature of the one or more unit materials supplied to the one or more second processing units; (q) a temperature in the one or more alkylation processing units; (r) a pressure in the one or more alkylation processing units; (s) a temperature in the one or more second processing units; or (t) a pressure in the one or more second processing units.
Clause 73. The alkylation processing assembly of clause 65, wherein the alkylation process controller is configured to: (a) control one or more process parameters associated with the alkylation process; and (b) predict, based at least in part on the one or more process parameters, a yield fraction associated with the one or more unit materials.
Clause 74. The alkylation processing assembly of clause 73, wherein: (a) the one or more unit materials comprises an alkylate product for use in blending gasoline; and (b) the prescriptive control comprises controlling an octane level associated with the alkylate product produced by the alkylation unit.
Clause 75. The alkylation processing assembly of clause 65, wherein one or more of: (a) the olefin feed comprises one or more of butylenes, pentenes, propylenes, or amylenes; (b) the paraffin feed comprises one or more of isobutane or isopentane; (c) one or more catalyst feeds processed by the one or more alkylation processing units comprises one or more of aluminum chloride, sulfuric acid, or hydrogen fluoride; or (d) the one or more unit materials comprise isooctane.
Clause 76. The alkylation processing assembly of clause 65, wherein the one or more second processing units comprise at least a portion of a gasoline blending operation.
Clause 77. The alkylation processing assembly of clause 65, wherein: (a) the reactor is positioned to receive the olefin feed and the paraffin feed; and (b) the prescriptive control comprises controlling one or more of: (i) content of the olefin feed supplied to the reactor; (ii) content of the paraffin feed supplied to the reactor; (iii) a temperature inside the reactor; (iv) a water content inside the reactor; (v) purity of the paraffin feed supplied to the reactor; or (vi) a ratio of the olefin feed to the paraffin feed supplied to the reactor.
Clause 78. The alkylation processing assembly of clause 65, wherein the alkylation process controller is configured to control: (a) one or more operating parameters associated with the depropanizer; (b) content of the one or more unit materials produced by the depropanizer; (c) one or more operating parameters associated with the deisobutanizer; (d) content of the one or more unit materials produced by the deisobutanizer; (e) one or more operating parameters associated with the debutanizer; (f) content of the one or more unit materials produced by the debutanizer; (g) one or more operating parameters associated with the isostripper; or (h) content of the one or more unit materials produced by the isostripper.
Clause 79. The alkylation processing assembly of clause 65, wherein: (a) the one or more unit materials comprise an isobutane recycle stream having an isobutane recycle purity; and (b) the prescriptive control comprises controlling, based at least in part on the isobutane recycle purity, a ratio of the olefin feed to the paraffin feed supplied to the one or more alkylation
Clause 80. The alkylation processing assembly of clause 65, wherein: (a) the one or more unit materials comprise a catalyst recycle stream having a catalyst recycle purity; and (b) the prescriptive control comprises controlling, based at least in part on the catalyst recycle purity, a supply of fresh catalyst to the one or more alkylation processing units.
Clause 81. The alkylation processing assembly of clause 65, wherein the alkylation process controller is configured to control one or more process parameters associated with the one or more alkylation processing units against operating constraints of the one or more alkylation processing units.
Clause 82. The alkylation processing assembly of clause 65, wherein: (a) the reactor comprises one or more columns positioned to separate reaction products into one or more intermediate process feedstocks; (b) the alkylation process controller is configured to predict one or more intermediate process feedstock sample properties associated with the one or more intermediate process feedstock samples; and (c) the prescriptive control is based at least in part on the one or more intermediate process feedstock sample properties.

This is a continuation-in-part of U.S. Non-Provisional application Ser. No. 17/652,431, filed Feb. 24, 2022, titled “METHODS AND ASSEMBLIES FOR DETERMINING AND USING STANDARDIZED SPECTRAL RESPONSES FOR CALIBRATION OF SPECTROSCOPIC ANALYZERS,” which claims priority to and the benefit of U.S. Provisional Application No. 63/153,452, filed Feb. 25, 2021, titled “METHODS AND ASSEMBLIES FOR DETERMINING AND USING STANDARDIZED SPECTRAL RESPONSES FOR CALIBRATION OF SPECTROSCOPIC ANALYZERS,” and U.S. Provisional Application No. 63/268,456, filed Feb. 24, 2022, titled “ASSEMBLIES AND METHODS FOR ENHANCING CONTROL OF FLUID CATALYTIC CRACKING (FCC) PROCESSES USING SPECTROSCOPIC ANALYZERS,” the disclosures of all of which are incorporated herein by reference in their entireties.

Furthermore, the scope of the present disclosure shall be construed to cover various modifications, combinations, additions, alterations, etc., above and to the above-described embodiments, which shall be considered to be within the scope of this disclosure. Accordingly, various features and characteristics as discussed herein may be selectively interchanged and applied to other illustrated and non-illustrated embodiment, and numerous variations, modifications, and additions further may be made thereto without departing from the spirit and scope of the present disclosure as set forth in the appended claims.

What is claimed is:

1. A method for enhancing control of an alkylation process associated with a refining operation, the method comprising:
analyzing, via a first spectroscopic analyzer to provide olefin feed sample spectra, an olefin feed sample of an olefin feed supplied to an alkylation unit positioned to perform at least a portion of the alkylation process, the olefin feed having one or more olefin feed properties;
analyzing, via one of the first spectroscopic analyzer or a second spectroscopic analyzer to provide paraffin feed sample spectra, a paraffin feed sample of a paraffin feed supplied to the alkylation unit, the paraffin feed having one or more paraffin feed properties;
predicting one or more olefin feed sample properties associated with the olefin feed based at least in part on the olefin feed sample spectra;
predicting one or more paraffin feed sample properties associated with the paraffin feed based at least in part on the paraffin feed sample spectra;
processing, via the alkylation unit, the olefin feed, the paraffin feed, and one or more catalysts to produce one or more corresponding unit materials, the one or more corresponding unit materials comprising one or more of intermediate materials or unit product materials;
analyzing a unit material sample, via one of the first spectroscopic analyzer, the second spectroscopic analyzer, or a third spectroscopic analyzer, to provide unit material sample spectra;
predicting one or more unit material sample properties associated with the unit material sample based at least in part on the unit material sample spectra; and
prescriptively controlling, during the alkylation process, via one or more controllers based at least in part on the one or more olefin feed sample properties, the one or more paraffin feed sample properties, and the one or more unit material sample properties, one or more of:
(a) one or more olefin feed properties associated with the olefin feed supplied to the alkylation unit;
(b) one or more paraffin feed properties associated with the paraffin feed supplied to the alkylation unit;
(c) one or more intermediate properties associated with the intermediate materials produced by the alkylation unit;
(d) one or more unit product properties associated with the unit product materials produced by the alkylation unit;
(e) operation of the alkylation unit;
(f) operation of one or more first processing units positioned upstream relative to the alkylation unit; or
(g) operation of one or more second processing units positioned downstream relative to the alkylation unit,
so that the prescriptively controlling causes the alkylation process to produce one or more of:
(i) one or more intermediate materials each having one or more properties within a range of one or more target properties of the one or more intermediate materials,
(ii) one or more unit product materials each having one or more properties within a range of one or more target properties of the one or more unit product materials, or
(iii) one or more downstream materials produced by the one or more second processing units, the one or more downstream materials each having one or more properties within a range of one or more target properties of the one or more downstream materials,
thereby to cause the alkylation process to achieve material outputs that more accurately and responsively converge on one or more of the target properties.

2. The method of claim 1, further comprising prescriptively controlling content of the one or more downstream materials via control of one or more of:
(a) content of the olefin feed supplied to the alkylation unit;
(b) content of the paraffin feed supplied to the alkylation unit;
(c) content of the one or more catalysts processed by the alkylation unit;
(d) the operation of the alkylation unit;
(e) content of the intermediate materials produced by the alkylation unit;
(f) content of the unit product materials produced by the alkylation unit;
(g) the operation of the one or more first processing units; or
(h) the operation of the one or more second processing units.

3. The method of claim 1, wherein the prescriptively controlling comprises operating a prescriptive analytical model configured to improve an accuracy of one or more of:
(a) the one or more olefin feed properties;
(b) the one or more paraffin feed properties;
(c) content of the one or more of the unit materials;
(d) purity of the one or more catalysts; or
(e) the downstream materials produced by the one or more second processing units.

4. The method of claim 1, wherein the prescriptively controlling operation of one or more of (a) the one or more first processing units, (b) the alkylation unit, or (c) the one or more second processing units, comprises:
comparing one or more of: (a) the one or more of the olefin feed sample properties, (b) the one or more paraffin feed sample properties, (c) the one or more the unit material sample properties, or (d) one or more properties of the one or more downstream materials, to one or more of:
(i) material properties of a material database;
(ii) one or more threshold values associated with operation of one or more of: (aa) the one or more first processing units, (bb) the alkylation unit, or (cc) the one or more second processing units; or
(iii) one or more respective target properties of one or more of (aa) the one or more unit materials, or (bb) the one or more target properties of the one or more downstream materials.

5. The method of claim 1, wherein the prescriptively controlling comprises controlling one or more process parameters associated with the alkylation unit, the one or more process parameters comprising one or more of:
(a) one or more olefin feed parameters associated with the olefin feed, the one or more olefin feed parameters comprising one or more of (i) a rate of supply of the olefin feed, (ii) a pressure associated with the olefin feed, or (iii) a temperature of the olefin feed;
(b) one or more paraffin feed parameters associated with the paraffin feed, the one or more of the paraffin feed parameters comprising one or more of: (i) a rate of supply of the paraffin feed, (ii) a pressure associated with the paraffin feed, or (iii) a temperature of the paraffin feed; or
(c) one or more catalyst parameters associated with the one or more catalysts, the one or more catalyst parameters comprising one or more of: (i) a rate of supply of the one or more catalysts, (ii) a pressure associate with the one or more catalysts, or (iii) a temperature of the one or more catalysts.

6. The method of claim 1, wherein one or more of:
(a) the analyzing of the olefin feed sample is performed on-line and in real-time;
(b) the analyzing of the olefin feed sample is performed off-line in a laboratory setting;
(c) the analyzing of the paraffin feed sample is performed on-line and in real-time;
(d) the analyzing of the paraffin feed sample is performed off-line in a laboratory setting;
(e) the analyzing of the unit material sample is performed on-line and in real-time; or
(f) the analyzing of the unit material sample is performed off-line and in a laboratory setting.

7. The method of claim 1, wherein one or more of:
(a) the predicting of the one or more olefin feed sample properties comprises predicting a boiling point associated with the olefin feed sample, and the method comprises controlling, based at least in part on the boiling point associated with the olefin feed sample, operation of one or more of: (i) the one or more first processing units, (ii) the alkylation unit, or the (iii) one or more second processing units;
(b) the predicting of the one or more paraffin feed sample properties comprises predicting a boiling point associated with the paraffin sample, and the method comprises controlling, based at least in part on the boiling point associated with the paraffin feed sample, operation of one or more of: (i) the one or more first processing units, (ii) the alkylation unit, or the (iii) one or more second processing units; or
(c) the predicting of the one or more corresponding unit materials comprises predicting a boiling point associated with the unit material sample, and the method comprises controlling, based at least in part on the boiling point associated with the unit material sample, operation of one or more of: (i) the one or more first processing units, (ii) the alkylation unit, or the (iii) one or more second processing units.

8. The method of claim 1, wherein:
(a) the alkylation unit comprises a reactor positioned to receive the olefin feed and the paraffin feed; and
(b) the prescriptively controlling comprises controlling one or more of:
(i) content of the olefin feed supplied to the reactor;
(ii) content of the paraffin feed supplied to the reactor;
(iii) a temperature inside the reactor;
(iv) a water content inside the reactor;
(v) purity of the paraffin feed supplied to the reactor; or
(vi) a ratio of the olefin feed to the paraffin feed supplied to the reactor.

9. The method of claim 1, wherein one or more of:
(a) the alkylation unit comprises a depropanizer, and the prescriptively controlling comprises controlling one or more of:
(i) one or more operating parameters associated with the depropanizer; or
(ii) content of the one or more unit materials produced by the depropanizer;
(b) the alkylation unit comprises a deisobutanizer, and the prescriptively controlling comprises controlling one or more of:
(i) one or more operating parameters associated with the deisobutanizer; or
(ii) content of the one or more unit materials produced by the deisobutanizer;
(c) the alkylation unit comprises a debutanizer, and the prescriptively controlling comprises controlling one or more of:
(i) one or more operating parameters associated with the debutanizer; or
(ii) content of the one or more unit materials produced by the debutanizer, or
(d) the alkylation unit comprises an isostripper, and the prescriptively controlling comprises controlling one or more of:
(i) one or more operating parameters associated with the isostripper; or
(ii) content of the one or more unit materials produced by the isostripper.

10. The method of claim 9, wherein:
(a) the alkylation unit comprises a depropanizer, and the one or more unit materials produced by the depropanizer comprise a propane product stream and a paraffin recycle stream; and
(b) the prescriptively controlling comprises controlling a ratio of the propane product stream to the paraffin recycle stream produced by the depropanizer.

11. The method of claim 1, wherein:
(a) the alkylation unit comprises a reactor and one or more acid settlers positioned to receive a feed from the reactor; and
(b) the prescriptively controlling comprises controlling one or more of:
(i) one or more operating parameters associated with the reactor;
(ii) purity of one or more acid catalysts supplied to the reactor;
(iii) a feed rate of one or more catalysts supplied to the reactor;
(iv) water content inside the reactor;
(v) content of unit materials associated with the feed from the reactor;
(vi) one or more operating parameters associated with the one or more acid settlers; or
(vii) content of unit materials produced by the one or more acid settlers.

12. The method of claim 1, wherein:
(a) the unit materials comprise a paraffin recycle stream having a paraffin recycle purity; and
(b) the prescriptively controlling comprises controlling, based at least in part on the paraffin recycle purity, a ratio of the olefin feed to the paraffin feed supplied to the alkylation unit.

13. The method of claim 1, wherein:
(a) the one or more unit materials comprises an alkylate product for use in blending gasoline; and
(b) the prescriptively controlling comprises controlling a ratio of the olefin feed to the paraffin feed supplied to the alkylation unit, thereby to control an octane level associated with the alkylate product produced by the alkylation unit.

14. The method of claim 1, wherein one or more of:
(a) the olefin feed comprises one or more of butylenes, pentylenes, propylenes, or amylenes;
(b) the paraffin feed comprises one or more of isobutane or isopentane;
(c) the one or more catalysts comprise one or more of aluminum chloride, sulfuric acid, or hydrogen fluoride; or
(d) the one or more unit materials comprise isooctane.

15. The method of claim 1, wherein one or more of the first spectroscopic analyzer, the second spectroscopic analyzer, or the third spectroscopic analyzer, comprises one of a near-infrared spectroscopic analyzer, a mid-infrared spectroscopic analyzer, a combination of a near-infrared spectroscopic analyzer and a mid-infrared spectroscopic analyzer, a Raman spectroscopic analyzer, or a nuclear magnetic resonance spectroscopic analyzer.

16. The method of claim 1, further comprising one or more of:
(a) prescriptively controlling one or more of a temperature of the olefin feed sample, a temperature of the paraffin feed, or a temperature of the unit material sample, thereby to substantially maintain one or more of a temperature of the olefin feed sample, the paraffin feed sample, or the unit material sample, respectively, within a respective temperature range; or
(b) applying one or more temperature correction factors to one or more of:
(i) the olefin feed sample spectra;
(ii) the paraffin feed sample spectra; or
(iii) the unit material sample spectra.

17. The method of claim 1, wherein the one or more of (a) the one or more olefin feed sample properties, (b) the one or more paraffin feed sample properties, (c) the one or more unit material sample properties, or (d) one or more downstream material properties of the one or more downstream materials, comprise a content ratio indicative of relative amounts of one or more hydrocarbon classes present in one or more of (a) the olefin feed sample, (b) the paraffin feed sample, (c) the unit material sample, or (d) a downstream material sample, respectively.

18. The method of claim 1, further comprising analyzing, via a second spectroscopic analyzer, one or more of: (a) the paraffin feed sample, (b) the unit material sample, or (c) a downstream material sample of the one or more downstream materials, the first spectroscopic analyzer and the second spectroscopic analyzer being calibrated to generate standardized spectral responses.

19. The method of claim 1, wherein the method further comprises:
determining one or more properties of the one or more downstream materials, the one or more downstream materials comprising blended gasoline including at least a portion of the one or more unit materials;
wherein the one or more properties comprise one or more of a Research Octane Number (RON), a Motor Octane Number (MON), Anti-Knock Index (AKI), a total aromatics content, a total butane content, an amount of isobutane, an amount of n-isobutane, a total olefins content, an amount of gasoline, an amount of propane, an amount of pentane, an amount of hydrogen sulfide, an amount of sulfur, an amount of water content, an aniline point, an amount of propane, an amount of benzene, an amount of toluene, an amount of xylene, total Benzene Toluene Xylene (BTX) content, total Benzene Toluene Ethylbenzene Xylene (BTEX) content, a vapor pressure, distillation properties/parameters, a drivability index, a density, a specific gravity, an American Petroleum Institute (API) gravity, an amount of alcohol, or an Ethanol (EtOH) kick prediction.

20. An alkylation process control assembly to enhance control of an alkylation process associated with a refining operation, the alkylation process control assembly comprising:
(a) a first spectroscopic analyzer positioned to:
(i) receive an olefin feed sample of an olefin feed supplied to an alkylation unit associated with the alkylation process, the olefin feed having one or more olefin feed properties; and
(ii) analyze the olefin feed sample to provide olefin feed sample spectra;
(b) a second spectroscopic analyzer positioned to:
(i) receive a paraffin feed sample of a paraffin feed supplied to the alkylation unit associated with the alkylation process, the paraffin feed having one or more paraffin feed properties; and
(ii) analyze the paraffin feed sample to provide paraffin feed sample spectra;
(c) a third spectroscopic analyzer positioned to:
(i) receive a unit material sample of one more unit materials produced by the alkylation unit associated with the alkylation process, the one or more unit materials comprising one or more of intermediate materials or unit product materials, the first spectroscopic analyzer, the second spectroscopic analyzer, and the third spectroscopic analyzer being calibrated to generate standardized spectral responses; and
(ii) analyze the unit material sample to provide unit material sample spectra; and
(d) an alkylation process controller in communication with the first spectroscopic analyzer, the second spectroscopic analyzer, and the third spectroscopic analyzer, the alkylation process controller configured to:
(i) predict one or more olefin feed sample properties associated with the olefin feed sample based at least in part on the olefin feed sample spectra;
(ii) predict one or more paraffin feed sample properties associated with the paraffin feed sample based at least in part on the paraffin feed sample spectra;
(iii) predict one or more unit material sample properties associated with the unit material sample based at least in part on the unit material sample spectra; and
(iv) prescriptively control, during the alkylation process, based at least in part on the one or more olefin feed sample properties, the one or more paraffin feed sample properties, and the one or more unit material sample properties, one or more of:
(aa) one or more olefin feed parameters associated with the olefin feed supplied to the alkylation unit;
(bb) the one or more olefin feed properties associated with the olefin feed supplied to alkylation unit;
(cc) one or more paraffin feed parameters associated with the paraffin feed supplied to the alkylation unit;
(dd) the one or more paraffin feed properties associated with the paraffin feed supplied to the alkylation unit;
(ee) one or more unit materials properties associated with the one or more unit materials;
(ff) operation of the alkylation unit; or
(gg) operation of one or more second processing units positioned downstream relative to the alkylation unit,
so that the prescriptively controlling during the alkylation process causes the alkylation process to produce one or more of:
(aa) one or more intermediate materials each having one or more properties within a selected range of one or more target properties of the one or more intermediate materials;
(bb) one or more unit materials each having one or more properties within a selected range of one or more target properties of the one or more unit materials; or
(cc) one or more downstream materials each having one or more properties within a selected range of one or more target properties of the one or more downstream materials,
thereby to cause the refining process to achieve material outputs that more accurately and responsively converge on one or more target properties.

21. The alkylation process control assembly of claim 20, wherein the alkylation process controller is configured to control one or more of:
(a) content of the olefin feed supplied to the alkylation unit;
(b) content of the paraffin feed supplied to the alkylation unit;
(c) content of one or more catalysts processed by the alkylation unit;
(d) content of the intermediate materials produced by the alkylation unit;
(e) content of the unit product materials produced by the alkylation unit;
(f) the operation of the alkylation unit; or
(h) the operation of one or more second processing units downstream of the alkylation unit.

22. The alkylation process control assembly of claim 20, wherein:
(a) the alkylation unit comprises a depropanizer, and the prescriptive control comprises controlling one or more of:
(i) one or more operating parameters associated with the depropanizer; or
(ii) content of the one or more unit materials produced by the depropanizer;
(b) the alkylation unit comprises a deisobutanizer, and the prescriptive control comprises controlling one or more of:
(i) one or more operating parameters associated with the deisobutanizer; or
(ii) content of the one or more unit materials produced by the deisobutanizer;
(c) the alkylation unit comprises a debutanizer, and the prescriptive control comprises controlling one or more of:
(i) one or more operating parameters associated with the debutanizer; or
(ii) content of the one or more unit materials produced by the debutanizer; or
(d) the alkylation unit comprises an isostripper, and the prescriptively controlling comprises controlling one or more of:
(i) one or more operating parameters associated with the isostripper; or
(ii) content of the one or more unit materials produced by the isostripper.

23. The alkylation process control assembly of claim 22, wherein the alkylation process controller is configured to control one or more of:
(a) the one or more operating parameters associated with the depropanizer against operating constraints of the depropanizer;
(b) the one or more operating parameters associated with the deisobutanizer against operating constraints of the deisobutanizer;
(c) the one or more operating parameters associated with the debutanizer against operating constraints of the debutanizer; or
(d) the one or more operating parameters associated with the isostripper against operating constraints of the isostripper.

24. The alkylation process control assembly of claim 20, wherein the alkylation process controller is configured to control, prior to the analyzing, one or more of:
(a) conditioning the olefin feed samples to provide a conditioned olefin feed sample;
(b) conditioning the paraffin feed sample to provide a conditioned paraffin feed sample; or
(c) conditioning the unit material sample to provide a conditioned unit material sample.

25. The alkylation process control assembly of claim 20, wherein the alkylation process controller is configured to numerically treat one or more of: a spectral response of the first spectroscopic analyzer, a spectral response of the second spectroscopic analyzer, or a spectral response of the third spectroscopic analyzer.

26. The alkylation process control assembly of claim 20, wherein one or more of the first spectroscopic analyzer, the second spectroscopic analyzer, or the third spectroscopic analyzer comprises one of a near-infrared spectroscopic analyzer, a mid-infrared spectroscopic analyzer, a combination of one or more near-infrared spectroscopic analyzers and a mid-infrared spectroscopic analyzers, a Raman spectroscopic analyzer, or a nuclear magnetic resonance spectroscopic analyzer.

27. An alkylation process controller to enhance control of an alkylation process associated with a refining operation, the alkylation process controller being in communication with one or more spectroscopic analyzers and one or more alkylation processing units, the alkylation process controller being configured to:
predict one or more olefin feed sample properties associated with an olefin feed sample based at least in part on olefin feed sample spectra generated by the one or more spectroscopic analyzers;
predict one or more paraffin feed sample properties associated with a paraffin feed sample based at least in part on paraffin feed sample spectra generated by the one or more spectroscopic analyzers;
predict one or more unit material sample properties associated with a unit material sample based at least in part on unit material sample spectra generated by the one or more spectroscopic analyzers, the one or more unit material sample properties being associated with one or more unit materials produced by the one or more alkylation processing units, and the one or more unit materials comprising one or more of intermediate materials or unit product materials; and
prescriptively control, during the alkylation process, based at least in part on the one or more olefin feed sample properties, the one or more paraffin feed sample properties, and the one or more unit material sample properties, one or more of:
(a) the one or more olefin feed properties associated with the olefin feed supplied to the one or more alkylation processing units;
(b) the one or more paraffin feed properties associated with the paraffin feed supplied to the one or more alkylation processing units;
(c) one or more catalyst feed properties associated with a catalyst feed supplied to the one or more alkylation processing units;
(d) one or more intermediate properties associated with the intermediate materials produced by one or more of the alkylation processing units;
(e) operation of the one or more alkylation processing units;
(f) one or more unit materials properties associated with the one or more unit materials produced by the one or more alkylation processing units; or
(g) operation of one or more second processing units positioned downstream relative to the one or more alkylation processing units,
so that the prescriptively controlling causes the refining process to produce one or more of:
(i) the one or more intermediate materials each having one or more properties within a selected range of one or more target properties of the one or more intermediate materials;
(ii) the one or more unit materials each having one or more properties within a selected range of one or more target properties of the one or more unit materials; or
(iii) one or more downstream materials produced by the second processing units each having one or more properties within a selected range of one or more target properties of the one or more downstream materials,
thereby to cause the refining process to achieve material outputs that more accurately and responsively converge on one or more of the target properties.
